(19) United States
(12) Patent Application Publication
(10) Pub. No.: US 2014/0075593 A1
(43) Pub. Date: Mar. 13, 2014

(54) FLUORESCENCE ACTIVATED CELL SORTING (FACS) ENRICHMENT TO GENERATE PLANTS

(71) Applicant: Dow AgroSciences LLC, Indianapolis, IN (US)

(72) Inventors: German Spangenberg, Bundoora (AU); Sareena Sahab, Mernda (AU); John Mason, Preston (AU)

(73) Assignee: Dow AgroSciences LLC, Indianapolis, IN (US)

(21) Appl. No.: 14/020,694

(22) Filed: Sep. 6, 2013

Related U.S. Application Data
(60) Provisional application No. 61/697,890, filed on Sep. 7, 2012.

(51) Int. Cl. C12N 15/82

(52) U.S. Cl. CPC C12N 15/8241
USPC 800/278; 435/421; 800/298

(57) ABSTRACT

An Engineered Transgene Integration Platform (ETIP) is described that can be inserted randomly or at targeted locations in plant genomes to facilitate rapid selection and detection of a GOI that is perfectly targeted (both the 3′ and 5′ ends) at the ETIP genomic location. One element in the invention is the introduction of specific double stranded breaks within the ETIP. In some embodiments, an ETIP is described using zinc finger nuclease binding sites, but may utilize other targeting technologies such as meganucleases, TALs, CRISPRs, or leucine zippers. Also described are compositions of, and methods for producing, transgenic plants wherein the donor or payload DNA expresses one or more products of an exogenous nucleic acid sequence (e.g. protein or RNA) that has been stably-integrated into an ETIP in a plant cell. In embodiments, the ETIP facilitates testing of gene candidates and plant expression vectors from ideation through Development phases.

### CROSS-REFERENCE TO RELATED APPLICATION

The present application claims priority to the benefit of U.S. Provisional Patent Application Ser. No. 61/697,890, filed Sep. 7, 2012, the disclosure of which is hereby incorporated by reference in its entirety.

### FIELD OF THE DISCLOSURE

The disclosure relates to the field of fluorescence activated cell sorting to generate plants. In a preferred embodiment, the disclosure describes FACS enrichment of edited, regenerable protoplasts to generate fertile edited plants.

### BACKGROUND

The Fluorescence Activated Cell Sorter (FACS) was invented in the late 1960s by Bonner, Sweet, Hulett, Herzenberg, and others to do flow cytometry and cell sorting of viable cells. Becton Dickinson Immunocytometry Systems introduced the commercial machines in the early 1970s. Fluorescence-activated cell sorting (FACS) is a specialized type of flow cytometry. It provides a method for sorting a heterogeneous mixture of biological cells into two or more containers, one cell at a time, based upon the specific light scattering and fluorescent characteristics of each cell. It is a useful scientific instrument, as it provides fast, objective and quantitative recording of fluorescent signals from individual cells as well as physical separation of cells of particular interest.

The cell suspension is entrained in the center of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell per droplet. Just before the stream breaks into droplets, the flow passes through a fluorescence measuring station where the fluorescent character of interest of each cell is measured. An electrical charging ring is placed just at the point where the stream breaks into droplets. A charge is placed on the ring based on the immediately prior fluorescence intensity measurement, and the opposite charge is trapped on the droplet as it breaks from the stream. The charged droplets then fall through an electrostatic deflection system that diverts droplets into containers based upon their charge. In some systems, the charge is applied directly to the stream, and the droplet breaking off retains charge of the same sign as the stream. The stream is then returned to neutral after the droplet breaks off.

A wide range of fluorophores can be used as labels in flow cytometry. Fluorophores, or simply “fluors,” are typically attached to an antibody that recognizes a target feature on or in the cell; they may also be attached to a chemical entity with affinity for the cell membrane or another cellular structure. Each fluorophore has a characteristic peak excitation and emission wavelength, and the emission spectra often overlap. Consequently, the combination of labels which can be used depends on the wavelength of the lamp(s) or laser(s) used to excite the fluorochromes and on the detectors available.

Fluorescence-activated cell sorting (FACS) provides a rapid means of isolating large numbers of fluorescently tagged cells from a heterogeneous mixture of cells. Collections of transgenic plants with cell type-specific expression of fluorescent marker genes such as green fluorescent protein (GFP) are ideally suited for FACS-assisted studies of individual cell types.

It has been demonstrated that flow cytometric analysis and fluorescence activated cell sorting (FACS) of plant protoplasts is practicable, moreover, this technique has yielded valuable results in a number of different fields of research (Harkins and Galbraith, 1984; Galbraith et al., 1995; Sheen et al., 1995). For instance, FACS of protoplasts from Arabidopsis plants expressing tissue-specific fluorescent protein markers has been used to examine both basal and environmentally stimulated transcriptional profiles in particular cell types (Birnbaum et al., 2003; Brady et al., 2007; Gifford et al., 2008; Dinneny et al., 2008) and flow cytometry has been employed to analyze reactive oxygen species production and programmed cell death tobacco protoplasts (Nicotiana tabacum; Lin et al., 2006). A broad selection of fluorescent tools is available to study a plethora of physiological parameters in plants, e.g., cis-regulatory elements fused to fluorescent proteins (Haseloff and Siemering, 2006), genetically-encoded molecular sensors (Looger et al., 2005) or dye-based sensors (Haugland, 2002) can be used in combination with cytometry to measure diverse biological processes. However, there are certain inefficiencies with this process due to the sensitivities of the assays and thus there is room for improvement.

### SUMMARY OF THE DISCLOSURE

A particular embodiment of the disclosure relates to a method for generating a plant from a population of plant cells by isolating a plant protoplast utilizing a polynucleotide of interest by providing a population of plant protoplasts having at least one protoplast comprising a polynucleotide of interest and a fluorescent marker, wherein the population is substantially free of plant protoplasts comprising the fluorescent marker and not comprising the polynucleotide of interest; wherein the plant protoplast is encapsulated by sodium alginate; separating the at least one protoplast comprising the polynucleotide of interest and the fluorescent marker from the remaining plant protoplasts in the population, thereby isolating a plant protoplast comprising the polynucleotide of interest; regenerating a plant from said isolated plant protoplast; and culturing said plant.

In another embodiment of the invention, there can be a plant regenerated by isolating a plant protoplast comprising a polynucleotide of interest integrated into the genome of the plant protoplast by providing a population of plant protoplasts having at least one protoplast comprising a polynucleotide of interest and a fluorescent marker; wherein the plant protoplast is encapsulated by sodium alginate; recovering microcalli from the population of protoplasts comprising the polynucleotide of interest and the fluorescent marker wherein the at least one protoplast comprises the polynucleotide of interest and the fluorescent marker has been transformed with the polynucleotide of interest and a polynucleotide encoding the fluorescent marker; regenerating a plant from said microcalli; and culturing said plant.

Alternative embodiments include methods for producing a transgenic plant, the method can include providing a population of plant protoplasts having at least one protoplast comprising a polynucleotide of interest and a fluorescent marker, wherein the at least one protoplast comprises a site-specific nuclease, such that the polynucleotide of interest is capable of being integrated in the genome of the at least one plant protoplast by homologous recombination at a recognition site of the site-specific nuclease and wherein the plant protoplast is encapsulated by sodium alginate; separating the at least one protoplast comprising the polynucleotide of interest and the fluorescent marker from the remaining plant protoplasts in the population; regenerating the transgenic plant from the at least one protoplast; and culturing said transgenic plant.

The foregoing and other features will become more apparent from the following detailed description of several embodiments, which proceeds with reference to the accompanying figures.

### BRIEF DESCRIPTION OF THE FIGURES

FIGS. 1A-1E: Shows a sequence alignment of FAD2 gene sequences, generated using ALIGNX®.

FIG. 2: Shows a phylogenetic tree of FAD2 gene sequences generated using JALVIEW® v 2.3 based on neighbor joining distances.

FIGS. 3A-3M′: Shows a sequence alignment of FAD3 gene sequences, generated using ALIGNX®.

FIG. 4: Shows a phylogenetic tree of FAD3 gene sequences generated using JALVIEW® v 2.3 based on neighbor joining distances. The labeled sequences correspond as follows: FAD3A′/A″ is described throughout this application as FAD3A′; Haplotype2 is described throughout the application as FAD3C′; Haplotype 1 is described throughout the application as FAD3C″; and, Haplotype 3 is described throughout the application as FAD3A″.

FIG. 5: Shows a plasmid map of pDAB 104010 which that is a representative Zinc Finger Nuclease expression cassette. The lay-out of this construct was similar for the other ZFN expression cassettes, wherein the Zinc Finger domains, 24828 and 24829, were exchanged with alternative Zinc Finger domains that are described above.

FIG. 6: is an example multiple line graph showing number of sequence reads per 10,000 sequence reads with deletions at the target ZFN site. The X axis on the graph denotes number of bases deleted, the Y axis denotes number of sequence reads and the Z axis denotes color-coded sample identity as described to the right of the graph. Specific example shown is for locus 1 of the FAD2 gene family that contains 3 target ZFN sites, A, B and C with the four gene family members and two control transfections assessed as control samples A and B.

FIG. 7: (A) Details of figure axis are as FIG. 6. The figure displays data from ZFN targeting locus 4 of the FAD2 gene family. The locus contains two ZFN sites and two requisite control transfections. FIG. 7:(B) Specific sequence context (SEQ ID NOs 471-480) surrounding the ZFN target site, identifying FAD2A and C containing tri-nucleotide repeats of C, T and G, leading to the observed increase in single base deletions through sequencing of the FAD2A and C loci.

FIG. 8: Shows a plasmid map of pDAS000130.

FIG. 9: Shows a plasmid map of pDAS000271.

FIG. 10: Shows a plasmid map of pDAS000272.

FIG. 11: Shows a plasmid map of pDAS000273.

FIG. 12: Shows a plasmid map of pDAS000274.

FIG. 13: Shows a plasmid map of pDAS000275.

FIG. 14: Shows a plasmid map of pDAS000031.

FIG. 15: Shows a plasmid map of pDAS000036.

FIG. 16: Shows a plasmid map of pDAS000037.

FIG. 17 illustrates an ETIP and payload nucleic acid configuration, as well as the product of the targeted Payload at the ETIP site in the plant cell genome.

FIG. 18 illustrates transformation of protoplast followed by FACS selection of targeted Payload DNA at the ETIP in the host line using reconstruction of truncated scorable and selectable markers at both the 3′ and 5′ ends.

FIGS. 19A and 19B: Illustrates the homology directed repair of the ETIP canola event which results from the double stranded DNA cleavage of the genomic locus by the Zinc Finger Nuclease (pDAS000074 or pDAS000075) and the subsequent integration of the Ds-red donor (pDAS000068, pDAS000070, or pDAS000072) into the ETIP locus of the canola chromosome. The integration of the donor into the genomic locus results in a fully functional, highly expressing Ds-red transgene.

FIG. 20: Shows the FACS sorting of canola protoplasts and the calculated transfection efficiency of canola protoplasts that were transfected with pDAS000031 (“pDAS31”). In addition, the FACS sorting results of untransformed canola protoplasts are provided as a negative control.

FIG. 21: Shows the FACS sorting of canola protoplasts and the calculated transfection efficiency of canola ETIP protoplast events which were transfected with pDAS000064/pDAS000074 (top graph) and pDAS000064/pDAS000075 (bottom graph).

FIG. 22: Shows the FACS sorting of canola protoplasts and the calculated transfection efficiency of canola ETIP protoplast events which were transformed with pDAS000068/pDAS000074 (top graph) and pDAS000068/pDAS000075 (bottom graph).

FIG. 23: Shows the FACS sorting of canola protoplasts and the calculated transfection efficiency of canola ETIP protoplast events which were transformed with pDAS000070/pDAS000074 (top graph) and pDAS000070/pDAS000075 (bottom graph).

FIG. 24: Shows the FACS sorting of canola protoplasts and the calculated transfection efficiency of canola ETIP protoplast events which were transformed with pDAS000072/pDAS000074 (top graph) and pDAS000072/pDAS000075 (bottom graph).

FIG. 25: Shows a plasmid map of pDAS000074.

FIG. 26: Shows a plasmid map of pDAS000075.

FIG. 27: Shows a plasmid map of pDAS000064.

FIG. 28: Shows a plasmid map of pDAS000068.

FIG. 29: Shows a plasmid map of pDAS000070.

FIG. 30: Shows a plasmid map of pDAS000072.

FIG. 31: Is a schematic showing binding sites of transgene target primers and probe for transgene copy number estimation assay.

FIG. 32: Shows a SEQUENCHER® file showing FAD2A ZFN DNA recognition domain (bcl2075_Fad2a-r272a2 and bcl2075_Fad2a-278a2), and binding sites of ZFN specific primers (FAD2A.UnE.F1 and FAD2A.UnE.R1) and endogenous primers (FAD2A/2C.RB.UnE.F1 and FAD2A/2C.RB.UnE.R1).

FIG. 33: Shows a schematic showing binding sites of endogenous and transgene target primers used in the detection of transgene integration at FAD2A via perfect HDR.

FIG. 34: Is a schematic showing where Kpn1 restriction endonuclease sites would occur in a perfectly edited FAD2A locus, and where FAD2a 5′, hph and FAD2A 3′ Southern probes bind.

FIG. 35: Shows the location and size of Kpn1 fragments, FAD2A 5′, hph, FAD2A 3′ probes and expected outcomes of Southern analysis for plants that have integration of ETIP at FAD2A locus via HDR.

FIG. 36: Shows representative data output from copy number estimation qPCR. The left hand column represents data obtained from a known T₀ transgenic plant with a single random transgene insert and is used as the calibrator sample to which all other samples are “normalized” against. The right hand column is a known T₀ transgenic plant with 5 transgene integrations. The insert copy numbers for both plants was determined using Southern analysis. The remaining columns provide copy number estimates for the putative transgenic plants. The labels below the columns correspond with the columns in the graph and can be used to determine the estimated copy number for each transgenic plant. When using the software to estimate copy numbers, wild-type plants, non-transformed control plants, and plasmid only controls do not result in a copy number as they do not possess a Cq for both the hph and HMG FY target.

### DETAILED DESCRIPTION

Transient transformation of protoplasts is a widely utilized tool in plant research that is swift and unproblematic. The technique can be used, for example, to monitor the regulation of promoter elements, to analyze gene expression or enzymatic activity in response to a variety of stimuli, to examine the roles of transcription factors or signal-transduction cascade components or to study the subcellular localization of proteins (Sheen, 2001; Yoo et al., 2007). As opposed to stable transformation of plants (Arabidopsis thaliana being the most commonly used platform), which generally takes months and requires the use of a transfecting agent (usually Agrobacterium tumefaciens), transfection of protoplasts can be achieved in just one day and entails only raw DNA and either a chemical- or electroporation-based transfection method. Additionally, transient transformation analyses can overcome problems encountered with stable over-expression such as pleiotropic developmental effects or nonviability, when a cell-based assay is appropriate. However, due to the fact that protoplast transformation efficiency is never 100%, results can be convoluted by the non-transformed cells.

Transformation efficiencies are often low and variable (e.g. Cummins et al., 2007; <10%) and depend on the employed method as well as properties of the protoplasts and DNA used. The present invention relates to the field of plant biotechnology, but can be used for all biological purposes. In particular, embodiments of the present invention relate to the generation of native or transgenic plant cell lines from a heterogeneous population of plant cells through flow cytometric sorting. Such plant cell line may either be a monocot or a dicot. As will be apparent for a skilled person, the invention also uses the plant cell line for the regeneration of whole fertile plants.

One of the embodiments of the present inventions relates to FACS based sensitivity selection wherein there is better selection to select for successfully transformed cells. It has previously been reported that transformation of a population of plant cells such as a plant suspension culture frequently results in transgenic cultures that heterogeneous and inconsistent expression levels. The present invention is primarily concern with the provision of a plant based system. The ability to isolate and grow single cells has numerous possible applications. For example, methods outlined herein have utility in the improvement of processes related to the productivity of plant cell cultures. However, this application has broad applicability for all cells.

Embodiments of the present invention include the use of flow cytometric sorting such as FACS technology to separate or isolate single, i.e. individualized protoplast that are prepared from a population of plant cells using materials and methods known in the art. These protoplasts can be transformed and are capable of 1) producing a fluorescent marker protein or polypeptide; 2) producing a desired product; and/or 3) surviving in the presence of a selection agent. Sorting criteria for FACS can be selected from the group comprising the genetic background (e.g., ploidy, aneuploidy), mutants, transgenics, gene exchange products, and fluorescence (autofluorescence (chloroplasts, metabolites), fluorescent proteins or enzyme mediation fluorescence). Any fluorescent protein may be used. A selection agent may or may not be used.

After separation or isolation of the single protoplasts by flow cytometric sorting, each single transformed protoplast is regenerated until the formation of a microcolony (microcallus) by co-cultivation. The plant source origin is not limited but is restricted to those lines, varieties and species whose protoplasts have the potential to regenerate until the formation of a microcolony or microcallus. The present invention will thus be applicable to all plant varieties and species for which a regeneration protocol has been established or will be provided. Thus, the present invention can be carried out with all plant varieties and species for which a regeneration portion has been established or will be provided for in the future.

The microcolony itself may be separated or removed from the feeder cell material and cultivated until the formation of a plant cell line.

Embodiments of the present invention can also include the generation of a callus tissue by 1) transferring the microcolony or microcallus to a solid cultivation medium and 2) cultivating the microcolony or microcallus in the presence of at least one selection agent until the formation of a transgenic callus tissue from which a transgenic plant cell line can be established by transferring the callus tissue to a liquid cultivation medium. The microcolony can also be removed or separated from the feeder cell material by mechanical means, i.e., by clone picking. In this case, no selection agent is needed and the cells comprised by the microcolony do not need to display resistance against any selection agent.

In some embodiments the cells can comprise a heterogeneous population of plant cells that are native or non-transgenic cells that, before being subjected to flow cytometric sorting and are stably or transiently transformed with at least one expression vector comprising at least one heterologous nucleic acid sequence which can be operably linked to a functional promoter, wherein said at least one heterologous nucleic acid sequence codes for a desired product. In additional embodiments the at least one heterologous nucleic acid sequence is operably linked to at least one functional promoter wherein the at least one heterologous nucleic acid sequence codes for a fluorescent marker protein or polypeptide and at least one heterologous nucleic acid sequence for resistance to a selection agent or for a desired product. Additional embodiments can include wherein the cells may additionally comprise a heterologous nucleic acid sequence that codes for a desired product to be accumulated in the transgenic plant cell line as provided.

In other embodiments the genome of the host cell can be expressed so that the recombinant protein or peptide can be modified by recombination, for example homologous recombination or heterologous recombination.

Any (transgenic) monoclonal or diclonal plant cell lines established can be treated or cultivated in the presence of precursors, inducers, hormones, stabilizers, inhibitors, RNAi/siRNA molecules, signaling compounds, enzymes and/or elicitors in addition to or instead of the vector suspension, for the production of recombinant proteins or metabolites.

Heterologous nucleic acids may encode genes of bacterial, fungal, plant or non-plant origin such as fusion proteins, and proteins of animal origin. Polypeptides produced may be utilized for producing polypeptides which can be purified therefrom for use elsewhere. Proteins that can be produced in a process of the invention include heterodimers, immunoglobulins, fusions antibodies and single chain antibodies. Furthermore, the above genes may be altered to produce proteins with altered characteristics.

Embodiments of the present invention include the ability to produce a large variety of proteins and polypeptides. These embodiments can also include methods for the production of at least one desired product selected from the group consisting of heterologous proteins or polypeptides, secondary metabolites, and markers. The method comprises to use the plant cell line as established according to the invention in order to produce and accumulate the at least one desired product which is subsequently obtained or isolated from the producing cells or from the cultivation medium.

Additional methods include methods of generating at least an extracellular heterologous protein comprising the steps of 1) stably introducing into a target cell comprised by the starting population of plant cells a first nucleic acid comprising the nucleotide sequence coding for the heterologous protein or desired product; 2) preparing protoplasts form plant suspension cells provided from said plant suspension culture, wherein the protoplasts are additionally transformed and capable of i) producing a fluorescent marker protein or polypeptide and ii) surviving in presence of a selection agent; 3) separating single transformed protoplasts by subjecting the preparation of protoplasts to FACS; 4) regenerating a separated single transformed protoplast until the formation of a microcolony or microcallus by co-cultivation in the presence of feeder cell material; 5) generating callus tissue by i) transferring the microcolony or microcallus to solid cultivation medium and ii) cultivating the microcolony or microcallus in the presence of at least one selection agent until the formation of a transgenic callus tissue; 6) establishing a transgenic plant cell line by transferring the callus tissue to liquid cultivation medium; 7) causing or permitting expression from the nucleic acid of the heterologous protein or desired product by providing appropriate cultivation conditions; and 8) harvesting the accumulated heterologous protein or desired product from the producing cells. Such isolation can be by entirely conventional means and may or may not entail partial or complete purification.

More than one gene may be used in each construct. Multiple vectors, each including one or more nucleotide sequences encoding heterologous protein of choice, may be introduced into the target cells as described herein or elsewhere. This can also be useful for producing multiple subunits of an enzyme.

The fluorescent marker protein or polypeptide can be a protein detectable by fluorescence such as GUS, fluorescent proteins such as GFP or DsRed, luciferase, etc. Preferably the reported is a non-invasive marker such as DsRed or GFP.

The techniques of this invention may be used to select for certain plants to be grown. Selection of a gene of interest may be handled in a number of ways. A large number of techniques are available for inserting DNA into a plant host cell. These techniques include transformation with T-DNA using Agrobacterium tumefaciens or Agrobacterium rhizogenes as transformation agent, fusion, injection, biolistics (microparticle bombardment), silicon carbide whiskers, aerosol beaming, PEG, or electroporation as well as other possible methods. If Agrobacteria are used for the transformation, the DNA to be inserted has to be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA. Intermediate vectors cannot replicate themselves in Agrobacteria. The intermediate vector can be transferred into Agrobacterium tumefaciens by means of a helper plasmid (conjugation). Binary vectors can replicate themselves both in E. coli and in Agrobacteria. They comprise a selection marker gene and a linker or polylinker which are framed by the right and left T-DNA border regions. They can be transformed directly into Agrobacteria (Holsters, 1978). The Agrobacterium used as host cell is to comprise a plasmid carrying a vir region. The vir region is necessary for the transfer of the T-DNA into the plant cell. Additional T-DNA may be contained. The bacterium so transformed is used for the transformation of plant cells. Plant explants can be cultivated advantageously with Agrobacterium tumefaciens or Agrobacterium rhizogenes for the transfer of the DNA into the plant cell. Whole plants can then be regenerated from the infected plant material (for example, pieces of leaf, segments of stalk, roots, but also protoplasts or suspension-cultivated cells) in a suitable medium, which may contain antibiotics or biocides for selection. The plants so obtained can then be tested for the presence of the inserted DNA. No special demands are made of the plasmids in the case of injection and electroporation. It is possible to use ordinary plasmids, such as, for example, pUC derivatives.

The transformed cells grow inside the plants in the usual manner. They can form germ cells and transmit the transformed trait(s) to progeny plants. Such plants can be grown in the normal manner and crossed with plants that have the same transformed hereditary factors or other hereditary factors. The resulting hybrid individuals have the corresponding phenotypic properties.

In some preferred embodiments of the invention, genes encoding proteins of interest are expressed from transcriptional units inserted into the plant genome. Preferably, said transcriptional units are recombinant vectors capable of stable integration into the plant genome and enable selection of transformed plant lines expressing mRNA encoding the proteins.

Once the inserted DNA has been integrated in the genome, it is relatively stable there (and does not come out again). It normally contains a selection marker that confers on the transformed plant cells resistance to a biocide or an antibiotic, such as kanamycin, G418, bleomycin, hygromycin, or chloramphenicol, inter alia. Plant selectable markers also typically can provide resistance to various herbicides such as glufosinate (e.g., PAT/bar), glyphosate (EPSPS), ALS-inhibitors (e.g., imidazolinone, sulfonylurea, triazolopyrimidine sulfonanilide, et al.), bromoxynil, HPPD-inhibitor resistance, PPO-inhibitors, ACC-ase inhibitors, and many others. The individually employed marker should accordingly permit the selection of transformed cells rather than cells that do not contain the inserted DNA. The gene(s) of interest are preferably expressed either by constitutive or inducible promoters in the plant cell. Once expressed, the mRNA is translated into proteins, thereby incorporating amino acids of interest into protein. The genes encoding a protein expressed in the plant cells can be under the control of a constitutive promoter, a tissue-specific promoter, or an inducible promoter.

Several techniques exist for introducing foreign recombinant vectors into plant cells, and for obtaining plants that stably maintain and express the introduced gene. Such techniques include the introduction of genetic material coated onto microparticles directly into cells (U.S. Pat. No. 4,945,050 to Cornell and U.S. Pat. No. 5,141,131 to DowElanco, now Dow AgroSciences, LLC). In addition, plants may be transformed using Agrobacterium technology, see U.S. Pat. No. 5,177,010 to University of Toledo; U.S. Pat. No. 5,104,310 to Texas A&M; European Patent Application 0131624B1; European Patent Applications 120516, 159418B1 and 176,112 to Schilperoot; U.S. Pat. Nos. 5,149,645, 5,469,976, 5,464,763 and 4,940,838 and 4,693,976 to Schilperoot; European Patent Applications 116718, 290799, 320500, all to Max Planck; European Patent Applications 604662 and 627752, and U.S. Pat. No. 5,591,616, to Japan Tobacco; European Patent Applications 0267159 and 0292435, and U.S. Pat. No. 5,231,019, all to Ciba Geigy, now Syngenta; U.S. Pat. Nos. 5,463,174 and 4,762,785, both to Calgene; and U.S. Pat. Nos. 5,004,863 and 5,159,135, both to Agracetus. Other transformation technology includes whiskers technology. See U.S. Pat. Nos. 5,302,523 and 5,464,765, both to Zeneca, now Syngenta. Other direct DNA delivery transformation technology includes aerosol beam technology. See U.S. Pat. No. 6,809,232. Electroporation technology has also been used to transform plants. See WO 87/06614 to Boyce Thompson Institute; U.S. Pat. Nos. 5,472,869 and 5,384,253, both to Dekalb; and WO 92/09696 and WO 93/21335, both to Plant Genetic Systems. Furthermore, viral vectors can also be used to produce transgenic plants expressing the protein of interest. For example, monocotyledonous plants can be transformed with a viral vector using the methods described in U.S. Pat. No. 5,569,597 to Mycogen Plant Science and Ciba-Geigy (now Syngenta), as well as U.S. Pat. Nos. 5,589,367 and 5,316,931, both to Biosource, now Large Scale Biology.

As mentioned previously, the manner in which the DNA construct is introduced into the plant host is not critical to this invention. Any method that provides for efficient transformation may be employed. For example, various methods for plant cell transformation are described herein and include the use of Ti or R1-plasmids and the like to perform Agrobacterium mediated transformation. In many instances, it will be desirable to have the construct used for transformation bordered on one or both sides by T-DNA borders, more specifically the right border. This is particularly useful when the construct uses Agrobacterium tumefaciens or Agrobacterium rhizogenes as a mode for transformation, although T-DNA borders may find use with other modes of transformation. Where Agrobacterium is used for plant cell transformation, a vector may be used which may be introduced into the host for homologous recombination with T-DNA or the Ti or Ri plasmid present in the host. Introduction of the vector may be performed via electroporation, tri-parental mating and other techniques for transforming gram-negative bacteria which are known to those skilled in the art. The manner of vector transformation into the Agrobacterium host is not critical to this invention. The Ti or Ri plasmid containing the T-DNA for recombination may be capable or incapable of causing gall formation, and is not critical to said invention so long as the vir genes are present in said host.

In some cases where Agrobacterium is used for transformation, the expression construct being within the T-DNA borders will be inserted into a broad spectrum vector such as pRK2 or derivatives thereof as described in Ditta et al. (1980) and EPO 0 120 515. Included within the expression construct and the T-DNA will be one or more markers as described herein which allow for selection of transformed Agrobacterium and transformed plant cells. The particular marker employed is not essential to this invention, with the preferred marker depending on the host and construction used.

For transformation of plant cells using Agrobacterium, explants may be combined and incubated with the transformed Agrobacterium for sufficient time to allow transformation thereof. After transformation, the Agrobacteria are killed by selection with the appropriate antibiotic and plant cells are cultured with the appropriate selective medium. Once calli are formed, shoot formation can be encouraged by employing the appropriate plant hormones according to methods well known in the art of plant tissue culturing and plant regeneration. However, a callus intermediate stage is not always necessary. After shoot formation, said plant cells can be transferred to medium which encourages root formation thereby completing plant regeneration. The plants may then be grown to seed and said seed can be used to establish future generations. Regardless of transformation technique, the gene encoding a bacterial protein is preferably incorporated into a gene transfer vector adapted to express said gene in a plant cell by including in the vector a plant promoter regulatory element, as well as 3′ non-translated transcriptional termination regions such as Nos and the like.

In addition to numerous technologies for transforming plants, the type of tissue that is contacted with the foreign genes may vary as well. Such tissue would include but would not be limited to embryogenic tissue, callus tissue types I, II, and III, hypocotyl, meristem, root tissue, tissues for expression in phloem, and the like. Almost all plant tissues may be transformed during dedifferentiation using appropriate techniques described herein.

As mentioned above, a variety of selectable markers can be used, if desired. Preference for a particular marker is at the discretion of the artisan, but any of the following selectable markers may be used along with any other gene not listed herein which could function as a selectable marker. Such selectable markers include but are not limited to aminoglycoside phosphotransferase gene of transposon Tn5 (Aph II) which encodes resistance to the antibiotics kanamycin, neomycin and G41; hygromycin resistance; methotrexate resistance, as well as those genes which encode for resistance or tolerance to glyphosate; phosphinothricin (bialaphos or glufosinate); ALS-inhibiting herbicides (imidazolinones, sulfonylureas and triazolopyrimidine herbicides), ACC-ase inhibitors (e.g., ayryloxypropionates or cyclohexanediones), and others such as bromoxynil, and HPPD-inhibitors (e.g., mesotrione) and the like.

In addition to a selectable marker, it may be desirous to use a reporter gene. In some instances a reporter gene may be used with or without a selectable marker. Reporter genes are genes that are typically not present in the recipient organism or tissue and typically encode for proteins resulting in some phenotypic change or enzymatic property. Examples of such genes are provided in Weising et al., 1988. Preferred reporter genes include the beta-glucuronidase (GUS) of the uidA locus of E. coli, the chloramphenicol acetyl transferase gene from Tn9 of E. coli, the green fluorescent protein from the bioluminescent jellyfish Aequorea victoria, and the luciferase genes from firefly Photinus pyralis. An assay for detecting reporter gene expression may then be performed at a suitable time after said gene has been introduced into recipient cells. A preferred such assay entails the use of the gene encoding beta-glucuronidase (GUS) of the uidA locus of E. coli as described by Jefferson et al. (1987), to identify transformed cells.

In addition to plant promoter regulatory elements, promoter regulatory elements from a variety of sources can be used efficiently in plant cells to express foreign genes. For example, promoter regulatory elements of bacterial origin, such as the octopine synthase promoter, the nopaline synthase promoter, the mannopine synthase promoter; promoters of viral origin, such as the cauliflower mosaic virus (35S and 19S), 35T (which is a re-engineered 35S promoter, see U.S. Pat. No. 6,166,302, especially Example 7E) and the like may be used. Plant promoter regulatory elements include but are not limited to ribulose-1,6-bisphosphate (RUBP) carboxylase small subunit (ssu), beta-conglycinin promoter, beta-phaseolin promoter, ADH promoter, heat-shock promoters, and tissue specific promoters. Other elements such as matrix attachment regions, scaffold attachment regions, introns, enhancers, polyadenylation sequences and the like may be present and thus may improve the transcription efficiency or DNA integration. Such elements may or may not be necessary for DNA function, although they can provide better expression or functioning of the DNA by affecting transcription, mRNA stability, and the like. Such elements may be included in the DNA as desired to obtain optimal performance of the transformed DNA in the plant. Typical elements include but are not limited to Adh-intron 1, Adh-intron 6, the alfalfa mosaic virus coat protein leader sequence, osmotin UTR sequences, the maize streak virus coat protein leader sequence, as well as others available to a skilled artisan. Constitutive promoter regulatory elements may also be used thereby directing continuous gene expression in all cells types and at all times (e.g., actin, ubiquitin, CaMV 35S, and the like). Tissue specific promoter regulatory elements are responsible for gene expression in specific cell or tissue types, such as the leaves or seeds (e.g., zein, oleosin, napin, ACP, globulin and the like) and these may also be used.

Promoter regulatory elements may also be active (or inactive) during a certain stage of the plant's development as well as active in plant tissues and organs. Examples of such include but are not limited to pollen-specific, embryo-specific, corn-silk-specific, cotton-fiber-specific, root-specific, seed-endosperm-specific, or vegetative phase-specific promoter regulatory elements and the like. Under certain circumstances it may be desirable to use an inducible promoter regulatory element, which is responsible for expression of genes in response to a specific signal, such as: physical stimulus (heat shock genes), light (RUBP carboxylase), hormone (Em), metabolites, chemical (tetracycline responsive), and stress. Other desirable transcription and translation elements that function in plants may be used. Numerous plant-specific gene transfer vectors are known in the art.

Plant RNA viral based systems can also be used to express bacterial protein. In so doing, the gene encoding a protein can be inserted into the coat promoter region of a suitable plant virus which will infect the host plant of interest. The protein can then be expressed thus providing protection of the plant from herbicide damage. Plant RNA viral based systems are described in U.S. Pat. No. 5,500,360 to Mycogen Plant Sciences, Inc. and U.S. Pat. Nos. 5,316,931 and 5,589,367 to Biosource.

Means of further increasing tolerance or resistance levels. It is shown herein that plants of the subject invention can be imparted with novel herbicide resistance traits without observable adverse effects on phenotype including yield. Such plants are within the scope of the subject invention. Plants exemplified and suggested herein can withstand 2×, 3×4× and 5× typical application levels, for example, of at least one subject herbicide. Improvements in these tolerance levels are within the scope of this invention. For example, various techniques are known in the art, and can foreseeably be optimized and further developed, for increasing expression of a given gene.

One such method includes increasing the copy number of the subject genes (in expression cassettes and the like). Transformation events can also be selected for those having multiple copies of the genes.

Strong promoters and enhancers can be used to “supercharge” expression. Examples of such promoters include the preferred 35T promoter which uses 35S enhancers. 35S, maize ubiquitin, Arabidopsis ubiquitin, A.t. actin, and CSMV promoters are included for such uses. Other strong viral promoters are also preferred. Enhancers include 4 OCS and the 35S double enhancer. Matrix attachment regions (MARs) can also be used to increase transformation efficiencies and transgene expression, for example.

Shuffling (directed evolution) and transcription factors can also be used for embodiments according to the subject invention.

Variant proteins can also be designed that differ at the sequence level but that retain the same or similar overall essential three-dimensional structure, surface charge distribution, and the like. See e.g. U.S. Pat. No. 7,058,515; Larson et al., Protein Sci. 2002 11: 2804-2813, “Thoroughly sampling sequence space: Large-scale protein design of structural ensembles”; Crameri et al., Nature Biotechnology 15, 436-438 (1997), “Molecular evolution of an arsenate detoxification pathway by DNA shuffling”; Stemmer, W. P. C. 1994, DNA shuffling by random fragmentation and reassembly: in vitro recombination for molecular evolution, Proc. Natl. Acad. Sci. USA 91: 10747-10751; Stemmer, W. P. C. 1994, Rapid evolution of a protein in vitro by DNA shuffling, Nature 370: 389-391; Stemmer, W. P. C. 1995, Searching sequence space. Bio/Technology 13: 549-553; Crameri, A., Cwirla, S, and Stemmer, W. P. C. 1996, Construction and evolution of antibody-phage libraries by DNA shuffling, Nature Medicine 2: 100-103; and Crameri, A., Whitehorn, E. A., Tate, E. and Stemmer, W. P. C., 1996, Improved green fluorescent protein by molecular evolution using DNA shuffling, Nature Biotechnology 14: 315-319.

The activity of recombinant polynucleotides inserted into plant cells can be dependent upon the influence of endogenous plant DNA adjacent the insert. Thus, another option is taking advantage of events that are known to be excellent locations in a plant genome for insertions. See e.g. WO 2005/103266 A1, relating to crylF and crylAc cotton events; FAD2, FAD3, wherein genes such as AAD1 or AAD12 or others can be substituted in those genomic loci in place of such inserts. Thus, targeted homologous recombination, for example, can be used according to the subject invention. This type of technology is the subject of for example, WO 03/080809 A2 and the corresponding published U.S. application (USPA 20030232410), relating to the use of zinc fingers for targeted recombination. The use of recombinases (cre-10x and flp-frt for example) is also known in the art.

Computational design of 5′ or 3′ UTR most suitable for synthetic hairpins can also be conducted within the scope of the subject invention. Computer modeling in general, as well as gene shuffling and directed evolution, are discussed elsewhere herein. More specifically regarding computer modeling and UTRs, computer modeling techniques for use in predicting/evaluating 5′ and 3′ UTR derivatives of the present invention include, but are not limited to: MFold version 3.1 available from Genetics Corporation Group, Madison, Wis. (see Zucker et al., Algorithms and Thermodynamics for RNA Secondary Structure Prediction: A Practical Guide. In RNA Biochemistry and Biotechnology, 11-43, J. Barciszewski & B. F. C. Clark, eds., NATO ASI Series, Kluwer Academic Publishers, Dordrecht, N L, (1999); Zucker et al., Expanded Sequence Dependence of Thermodynamic Parameters Improves Prediction of RNA Secondary Structure. J. Mol. Biol. 288, 911-940 (1999); Zucker et al., RNA Secondary Structure Prediction. In Current Protocols in Nucleic Acid Chemistry, S. Beaucage, D. E. Bergstrom, G. D. Glick, and R. A. Jones eds., John Wiley & Sons, New York, 11.2.1-11.2.10, (2000)), COVE (RNA structure analysis using covariance models (stochastic context free grammar methods)) v. 2.4.2 (Eddy & Durbin, Nucl. Acids Res. 1994, 22: 2079-2088) which is freely distributed as source code and which can be downloaded by accessing the website genetics.wust1.edu/eddy/software/, and FOLDALIGN, also freely distributed and available for downloading at the website bioinf.au.dk. FOLDALIGN/ (see Finding the most significant common sequence and structure motifs in a set of RNA sequences. J. Gorodkin, L. J. Heyer and G. D. Stormo. Nucleic Acids Research, Vol. 25, no. 18 pp 3724-3732, 1997; Finding Common Sequence and Structure Motifs in a set of RNA Sequences. J. Gorodkin, L. J. Heyer, and G. D. Stormo. ISMB 5; 120-123, 1997).

Embodiments of the subject invention can be used in conjunction with naturally evolved or chemically induced mutants (mutants can be selected by screening techniques, then transformed with other genes). Plants of the subject invention can be combined with various resistance genes and/or evolved resistance genes. Traditional breeding techniques can also be combined with the subject invention to powerfully combine, introgress, and improve selection of traits.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

### EXAMPLES

The following Examples are provided to illustrate certain particular features and/or aspects. These Examples should not be construed to limit the disclosure to the particular features or aspects described.

### Example 1

### Identification of Paralogous Fad2 and Fad3 Target Sequences from a Bacterial Artificial Chromosome Library

BAC Construction

A Bacterial Artificial Chromosome (BAC) library was sourced from a commercial vendor (Amplicon Express, Pullman, Wash.). The BAC library consisted of 110,592 BAC clones containing high molecular weight genomic DNA (gDNA) fragments isolated from Brassica napus L. var. DH10275. The gDNA was digested with either the BamHI or HinDIII restriction enzyme. Isolated gDNA fragments of about 135 Kbp were ligated into the pCC1BAC vector (Epicentre, Madison, Wis.) and transformed into Escherichia coli str. DH10B (Invitrogen). The BAC library was made up of an even number of BAC clones that were constructed using the two different restriction enzymes. As such, the Hind III constructed BAC library consisted of 144 individual 384-well plates. Likewise, the BamHI constructed BAC library consisted of 144 individual 384-well plates. A total of 110,592 BAC clones were isolated and arrayed into 288 individual 384-well plates. Each of the 288 individual 384 well plates were provided by the vendor as a single DNA extraction for rapid PCR based screening. The resulting BAC library covers approximately 15 Gbp of gDNA, which corresponds to a 12-fold genome coverage of Brassica napus L. var. DH10275genome (estimate of the Brassica napus L. genome is ca. 1.132 Gbp as described in Johnston et al. (2005) Annals of Botany 95:229-235).

Sequence Analysis of Fad2 Coding Sequences Isolated From the BAC Library

The constructed BAC library was used to isolate FAD2 gene coding sequences. Sequencing experiments were conducted to identify the specific gene sequences of four FAD2 gene paralogs from Brassica napus L. var. DH10275.

The FAD2 gene sequence was initially identified within the model species Arabidopsis thaliana. The gene sequence is listed in Genbank as Locus Tag: At3g12120. Comparative genomic relationships between the model plant species Arabidopsis thaliana and the diploid Brassica rapa, one of the progenitors of the tetraploid Brassica napus, have been previously described. (Schranz et al. (2006) Trends in Plant Science 11(11):535-542). With specific relation to the FAD2 gene the comparative analysis predicted that 3-4 copies of the gene may occur within the diploid Brassica genome. Additional genetic mapping studies were completed by Scheffler et al. (1997) Theoretical and Applied Genetics 94; 583-591. The results of these genetic mapping studies indicated that four copies of the FAD2 gene were present in Brassica napus.

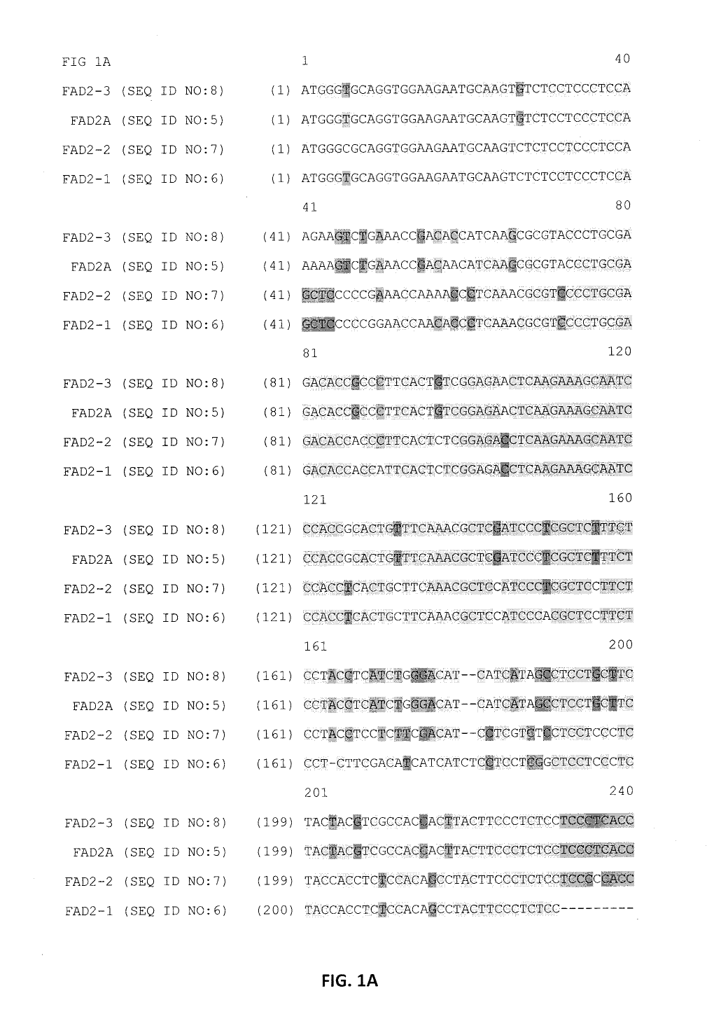

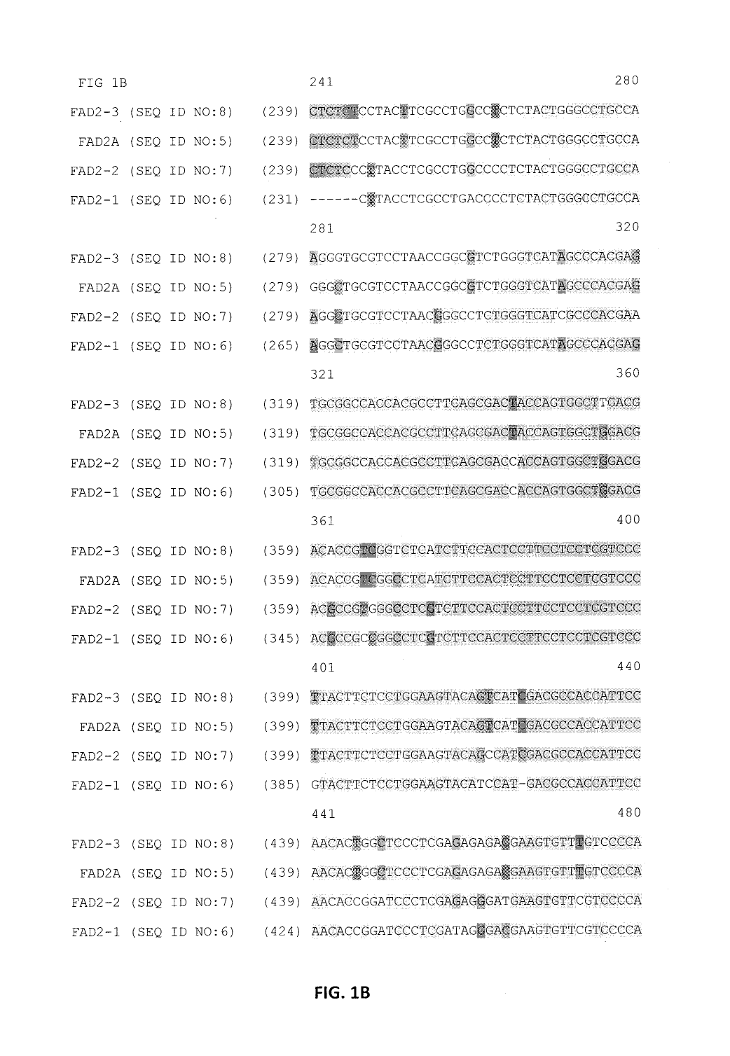

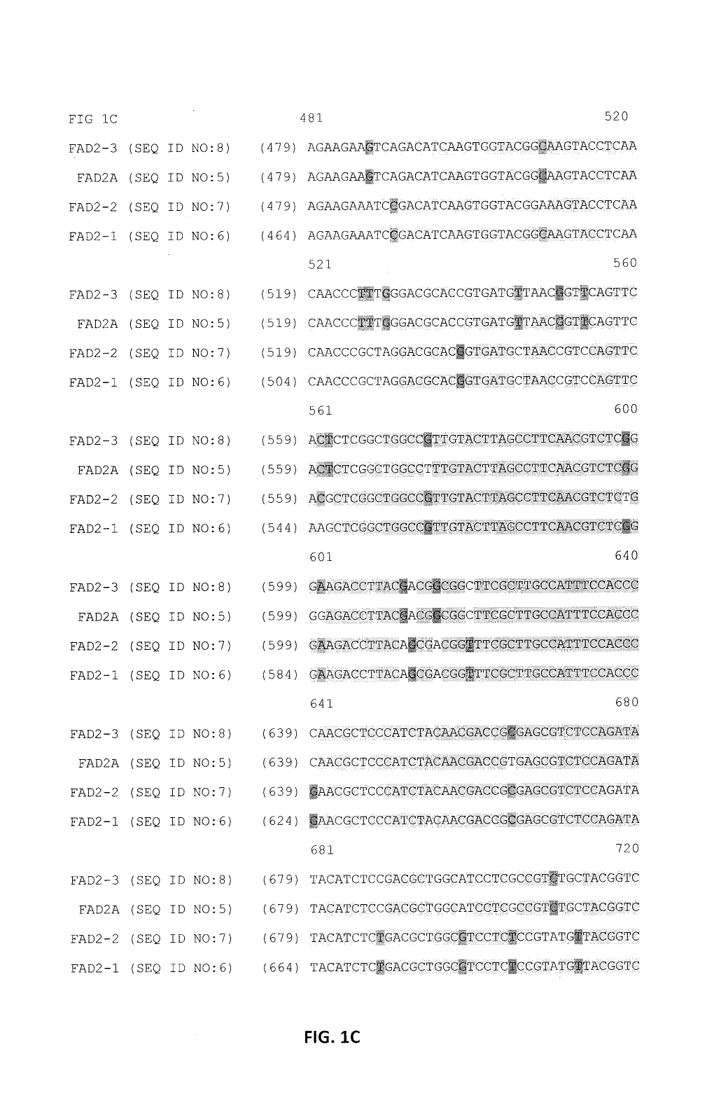

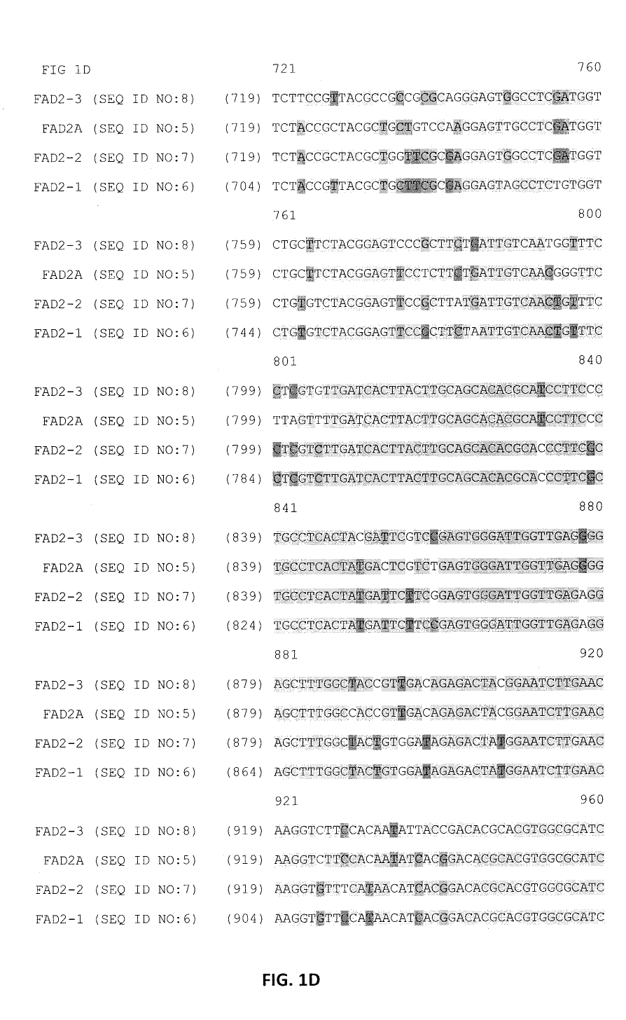

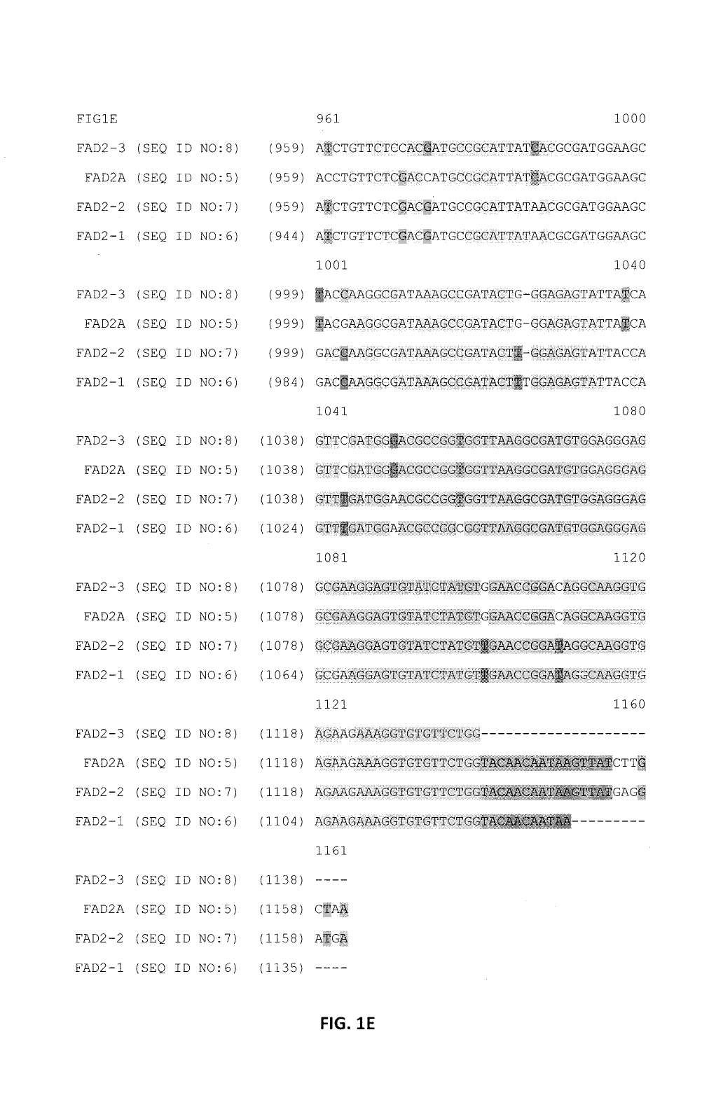

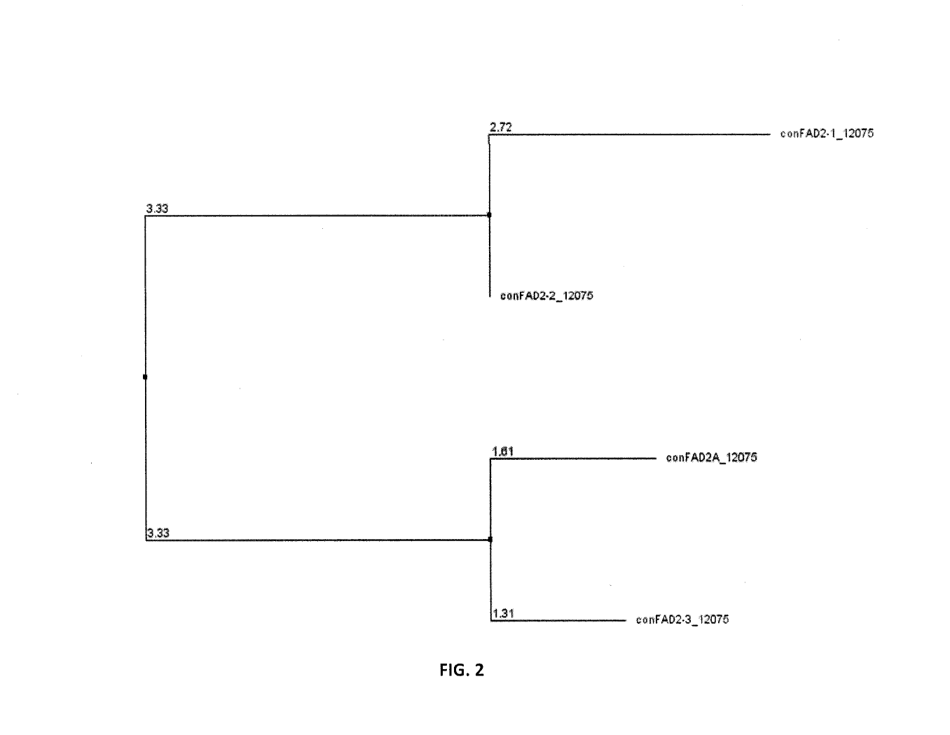

Sequencing analysis of the BAC library which was constructed from B. napus L. var. DH12075 resulted in the isolation of four BAC sequences (SEQ ID NO:1, SEQ ID NO:2, SEQ ID NO:3, and SEQ ID NO:4) from which the coding sequences for the FAD2A (SEQ ID NO:5), FAD2-1 (SEQ ID NO:6), FAD2-2 (SEQ ID NO:7), and FAD2-3(SEQ ID NO:8) genes were determined. The FAD2A, FAD2-1, FAD2-2, and FAD2-3 gene sequences were identified and genetically mapped. Sequence analysis of the four FAD2 genes was conducted using a sequence alignment program and a neighbor-joining tree using percentage of identity. The sequence alignment was made via the ALIGNX® program from the Vector NTI Advance 11.0 computer program (Life Technologies, Carlsbad, Calif.) and is shown in FIG. 1. ALIGNX® uses a modified Clustal W algorithm to generate multiple sequence alignments of either protein or nucleic acid sequences for similarity comparisons and for annotation. The neighbor-joining tree was created with JALVIEWv2.3® software and is shown in FIG. 2. (Waterhouse et al. (2009) Bioinformatics 25 (9) 1189-1191). As shown in FIG. 2, the analysis of the isolated sequences indicated that the FAD2A and FAD2-3 sequences shared high levels of sequence similarity and that, likewise, FAD2-1 and FAD2-2 shared high levels of sequence similarity. The four sequences can be categorized in two clades, wherein FAD2A and FAD2-3 comprise a first Glade, and FAD2-1 and FAD2-2 comprise a second Glade.

Next, the newly isolated FAD2 sequences from Brassica napus were used to BLAST genomic libraries isolated from a Brassica rapa genomic BAC library and Brassica oleracea shotgun genomic sequence reads. Both, Brassica rapa and Brassica oleracea are diploid progenitors of Brassica napus which is an amphidiploid species (AC genome, n=19). Brassica napus derived from a recent hybridization event between Brassica rapa (A sub-genome, n=10) and Brassica oleracea (C sub-genome, n=9). The diploid progenitor sequences were compared to the four different FAD2 coding sequences isolated from Brassica napus using a BLASTn analysis. This sequence analysis identified specific, annotated gene sequences from Brassica rapa and Brassica oleracea which shared the highest sequence similarity to the newly discovered Brassica napus FAD2 sequences. Table 1 lists the newly identified FAD2 coding sequence and the corresponding progenitor reference sequence accession number and source organism.

TABLE 1
FAD2 sequences from Brassica napus and the corresponding
progenitor organism and related FAD sequence accession number.

| Isolated | | |
|---|---|---|
| gene sequence | Progenitor organism and sequence accession number | |
| FAD2A | B. rapa | Genbank Accession No: KBrB063G23 |
| | | (A05) |
| FAD2-3 | B. oleracea | Genbank Accession No: GSS23580801* |
| FAD2-1 | B. rapa | Genbank Accession No: KBrB130I19 |
| FAD2-2 | B. oleracea | Genbank Accession No: GSS 17735412 |

*The Genbank sequence entry was edited

The FAD2 genes exist in the Brassica napus genome as two copies of each gene per sub-genome. One copy of each gene is located on the A sub-genome, and likewise one copy of each gene is located on the C sub-genome. New naming conventions are described to indicate which sub-genome that each gene is located on. The high levels of sequence similarity between the four different FAD2 coding sequences isolated from the Brassica napus BAC genomic DNA library and the progenitor sequence data suggest that FAD2-3 is a duplicate of the FAD2 sequence from the C sub-genome and could be relabeled as FAD2C; FAD2-1 is a duplicate of the FAD2 sequence from the A sub-genome and could therefore be labeled as FAD2A′; and finally, FAD2-2 is a second copy that was duplicated from the FAD2 sequence of the C sub-genome and could be labeled as FAD2C′.

Sequence Analysis of Fad3 Coding Sequences Isolated From the BAC library

The constructed BAC library was used to isolate FAD3 gene coding sequences. Sequencing experiments were conducted to identify the specific gene sequences of five FAD3 gene paralogs from Brassica napus L. var. DH10275.

The FAD3 gene sequence was initially identified within the model species Arabidopsis thaliana. The gene sequence is listed in Genbank as Locus Tag: At2g29980. Comparative genomic relationships between the model plant species Arabidopsis thaliana and the diploid Brassica rapa, one of the progenitors of the tetraploid Brassica napus, have been previously described. (Schram et al. (2006) Trends in Plant Science 11(11):535-542). With specific relation to the FAD gene the comparative analysis predicted that 3-4 copies of the gene may occur within the diploid Brassica genome. Additional genetic mapping studies were completed by Scheffler et al. (1997) Theoretical and Applied Genetics 94; 583-591. The results of these genetic mapping studies indicated that six copies of the FAD3 gene were present in Brassica napus.

Previous sequencing efforts focused on the FAD3 genes from Brassica napus had identified and genetically mapped both A and C genome specific copies (Hu et al., (2006) Theoretical and Applied Genetics, 113(3): 497-507). A collection of EST sequences from seed specific cDNA libraries had previously been constructed and sequenced from the plant line DH12075 by Andrew Sharpe of Agriculture and Agri-food Canada, 107 Science Place, Saskatoon, Saskatchewan. As a collection of ESTs from the doubled haploid canola plant DH12075 full length gene sequences were not available, moreover the indications of sequence quality and confidence of correctly called nucleotides was also not available. Consequently, sequence variation between different FAD gene sequence reads could not be unequivocally attributed to different gene copies of the various paralogs of the FAD3 gene family, nor was the genomic sequence available. However, when a combined sequence analysis was performed with the ESTs as well as the two FAD3A and FAD3C full length gene sequences described in Hu et al., (2006), ESTs that matched both of the genes were identified along with an additional 3 haplotypes. As a result, a total of six unique haplotypes of FAD3 were identified. Following the assembly of all available data for the various FAD3 haplotypes, high levels of exon sequence divergence in exon 1 was identified. The divergence of the FAD3 sequence in exon 1 was identified as an opportunity which could be utilized for the design of gene/allele specific PCR primers. In addition, exons were identified that were either minimally differentiated between haplotypes (e.g., exons 5, 6, 7 and 8 had 1-3 bp that varied between FAD3A and FAD3C) or that were devoid of sequence variation (e.g., exons 2 and 3).

Sequencing analysis of the BAC library which was constructed from B. napus L. var. DH12075 resulted in the isolation of six BAC sequences (SEQ ID NO:9, SEQ ID NO:10, SEQ ID NO:11, SEQ ID NO:12, SEQ ID NO:13, and SEQ ID NO:14) from which the coding sequences for the FAD3A (SEQ ID NO:15), FAD3A′ (SEQ ID NO:16), FAD3A″ (SEQ ID NO:17), FAD3C (SEQ ID NO:18), FAD3C″ (SEQ ID NO:19), and FAD3C′ (SEQ ID NO:20) genes were determined. The FAD3A, FAD3A′, FAD3A″, FAD3C, FAD3C″, and FAD3C′ gene sequences were identified and genetically mapped.

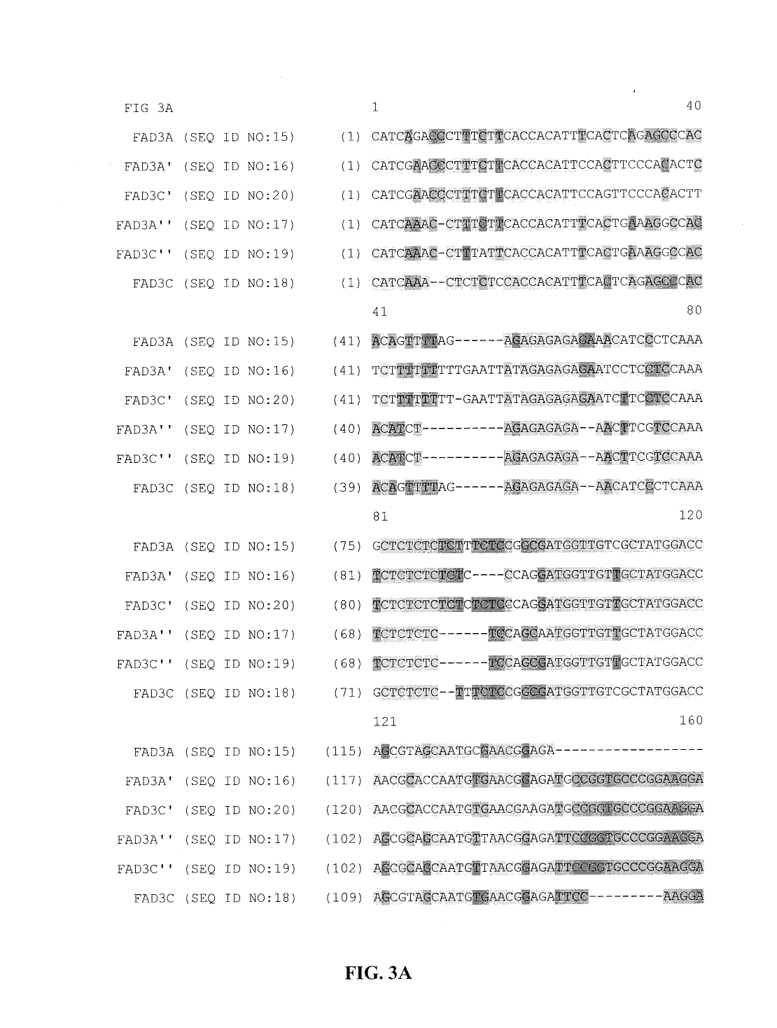

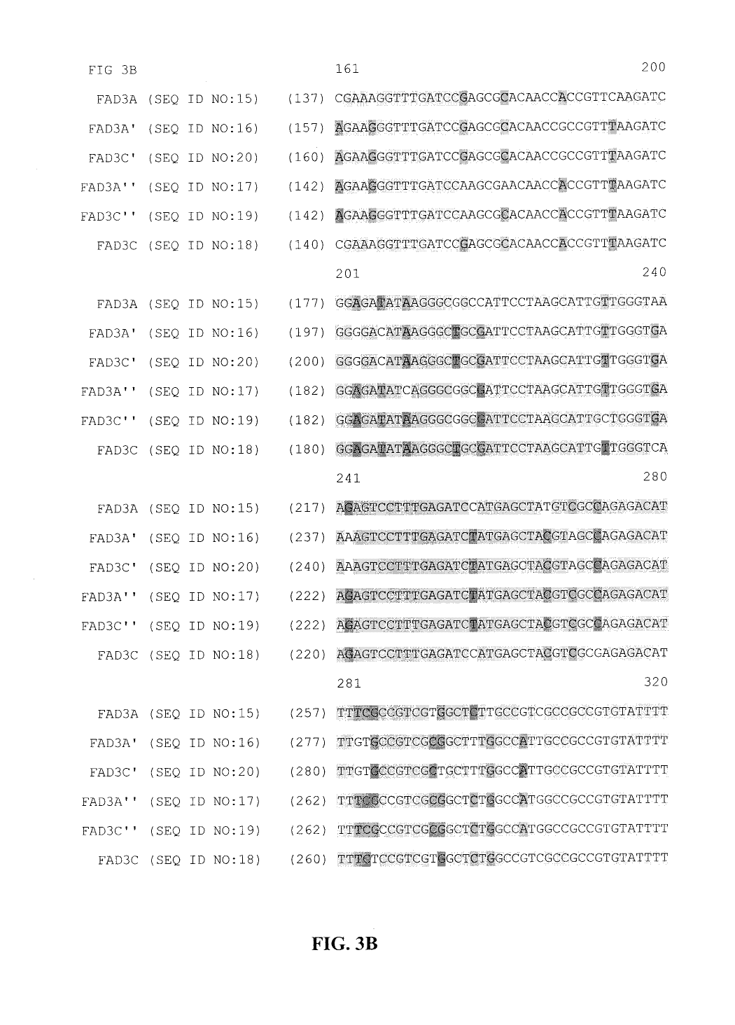

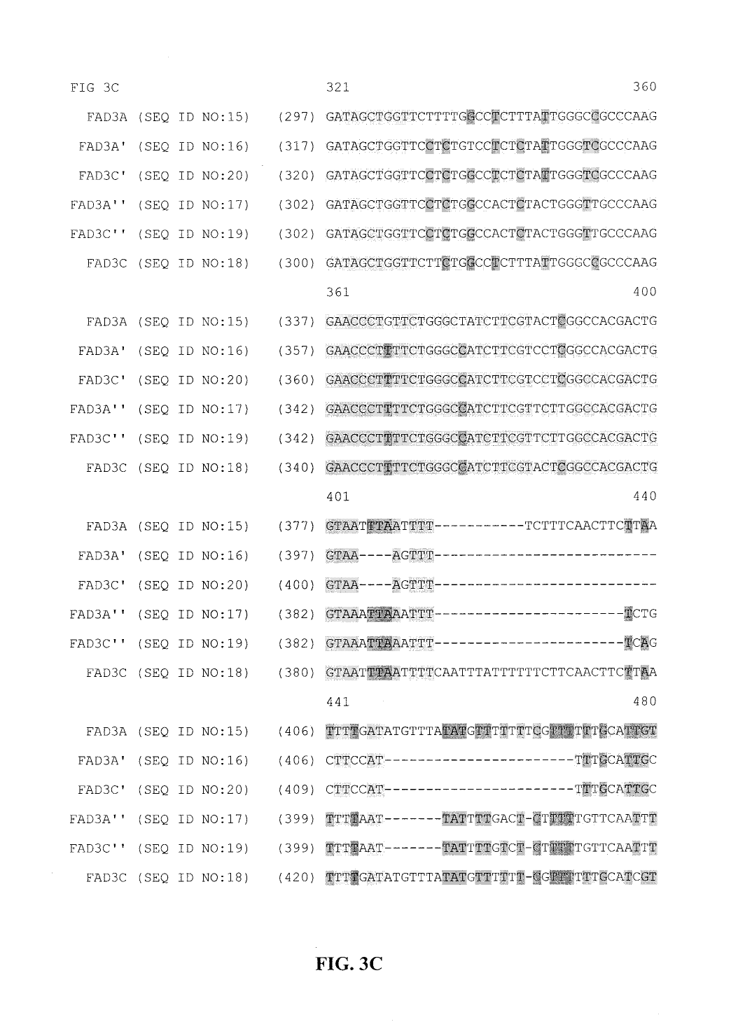

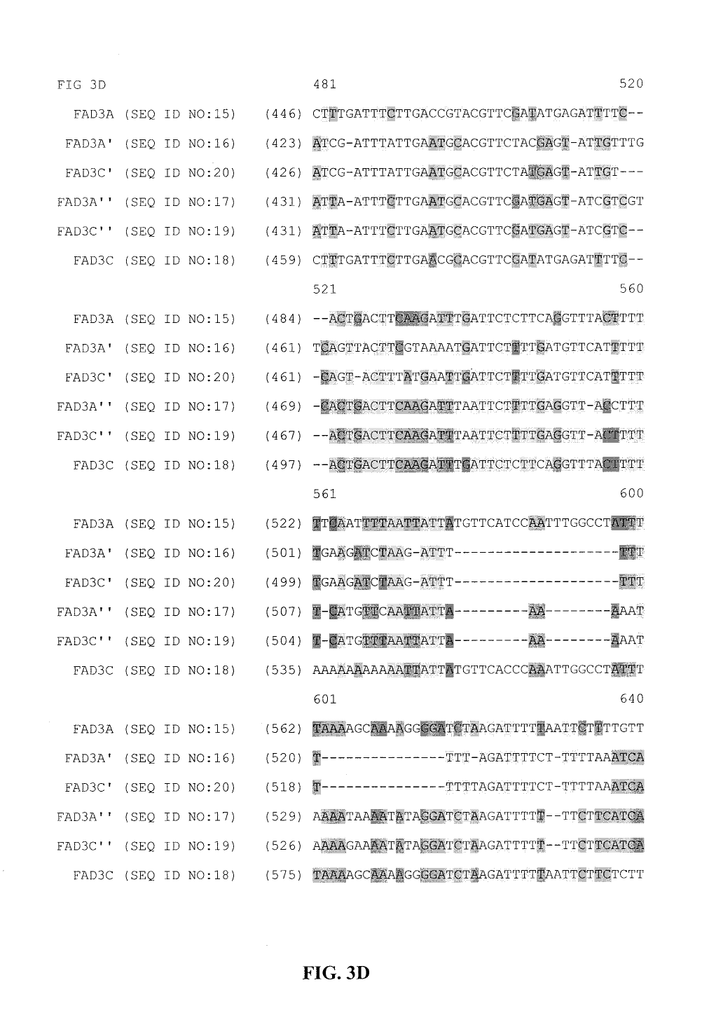

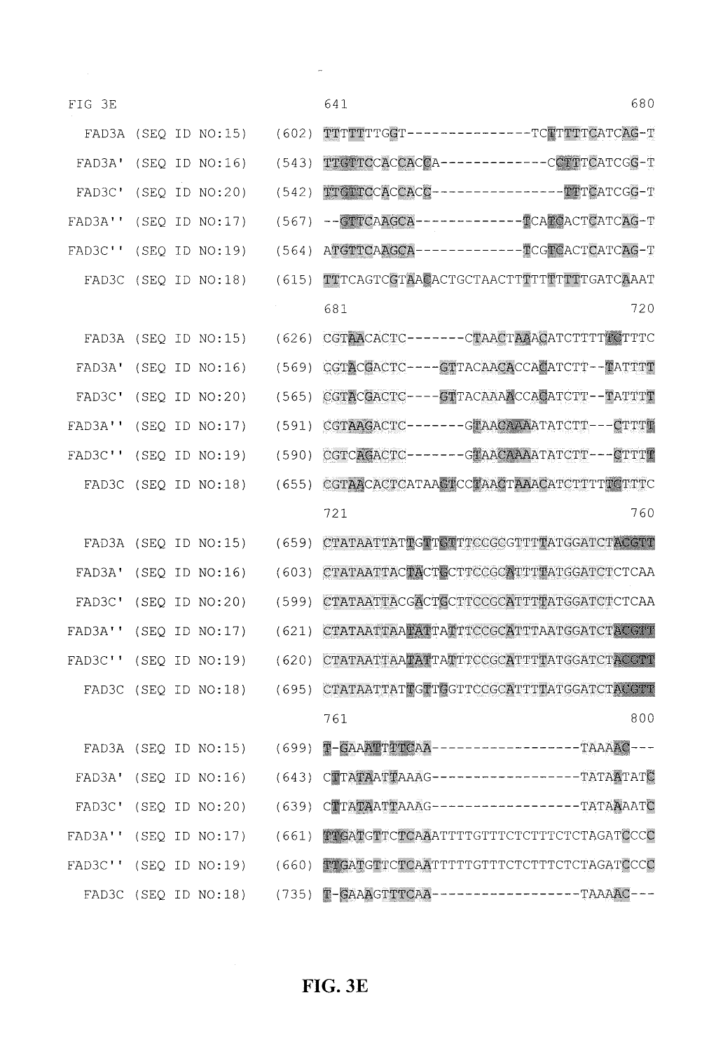

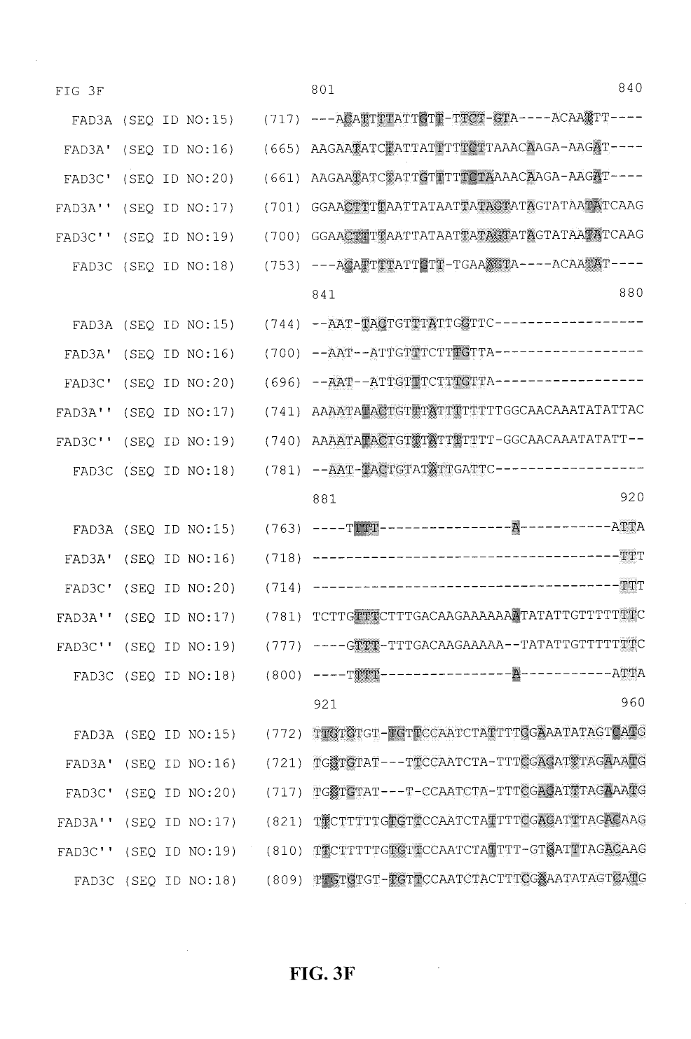

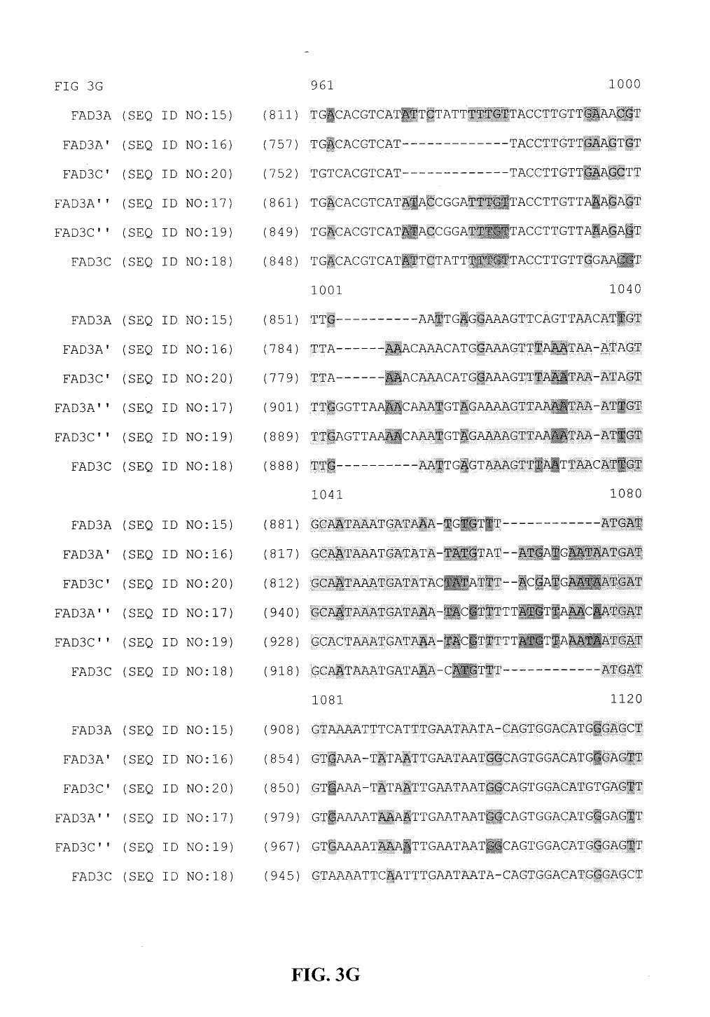

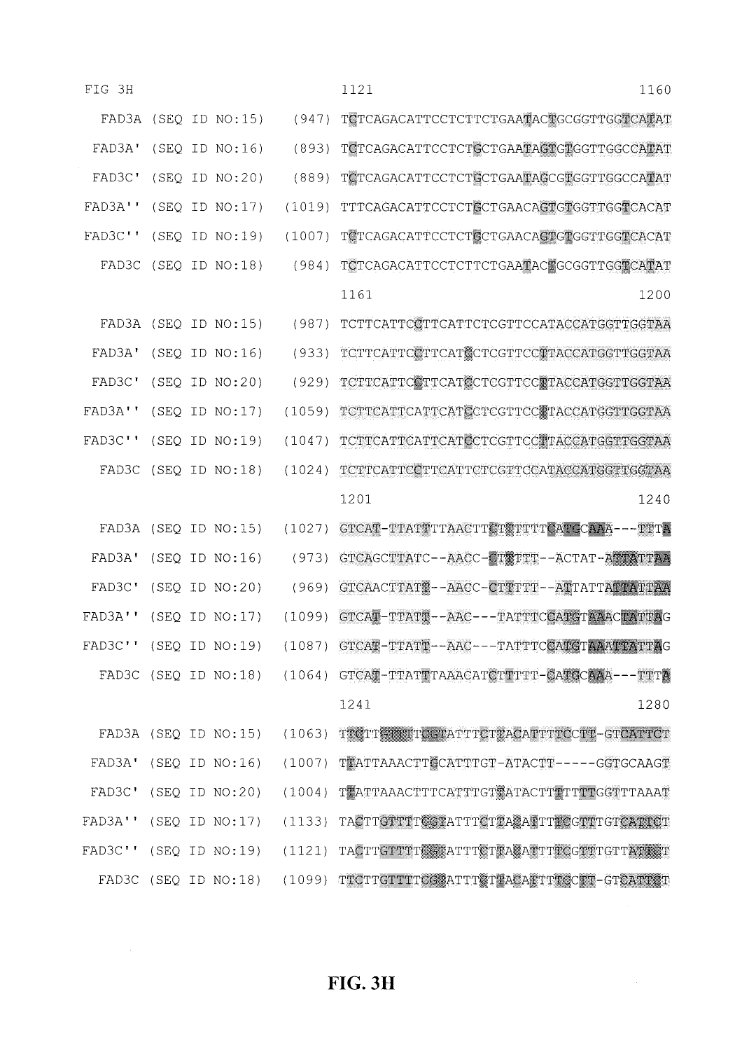

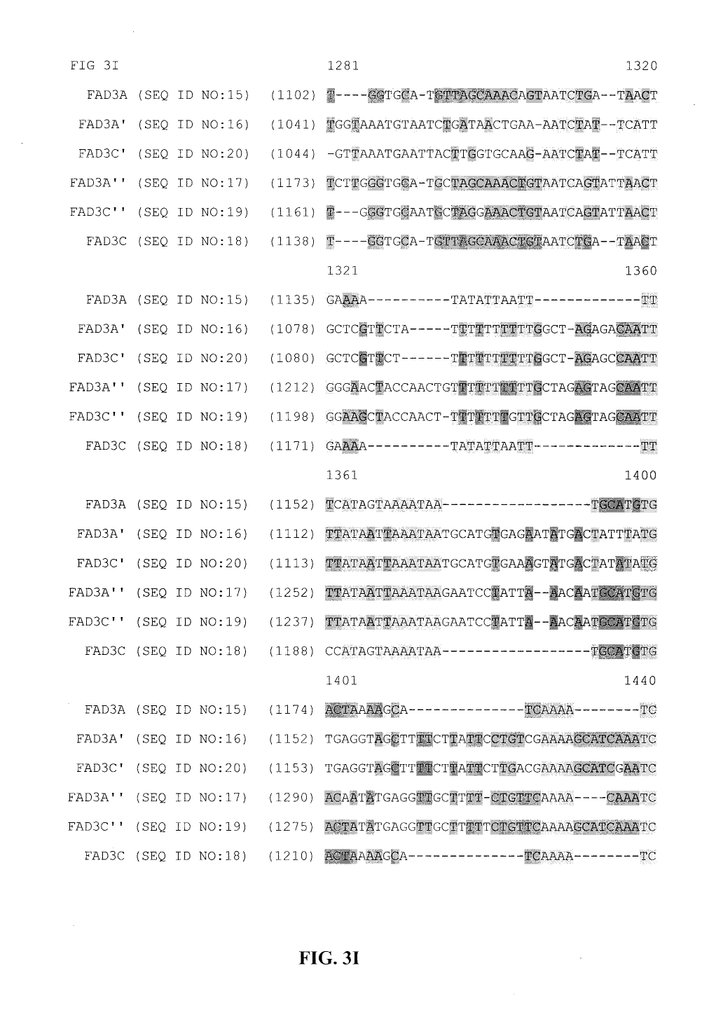

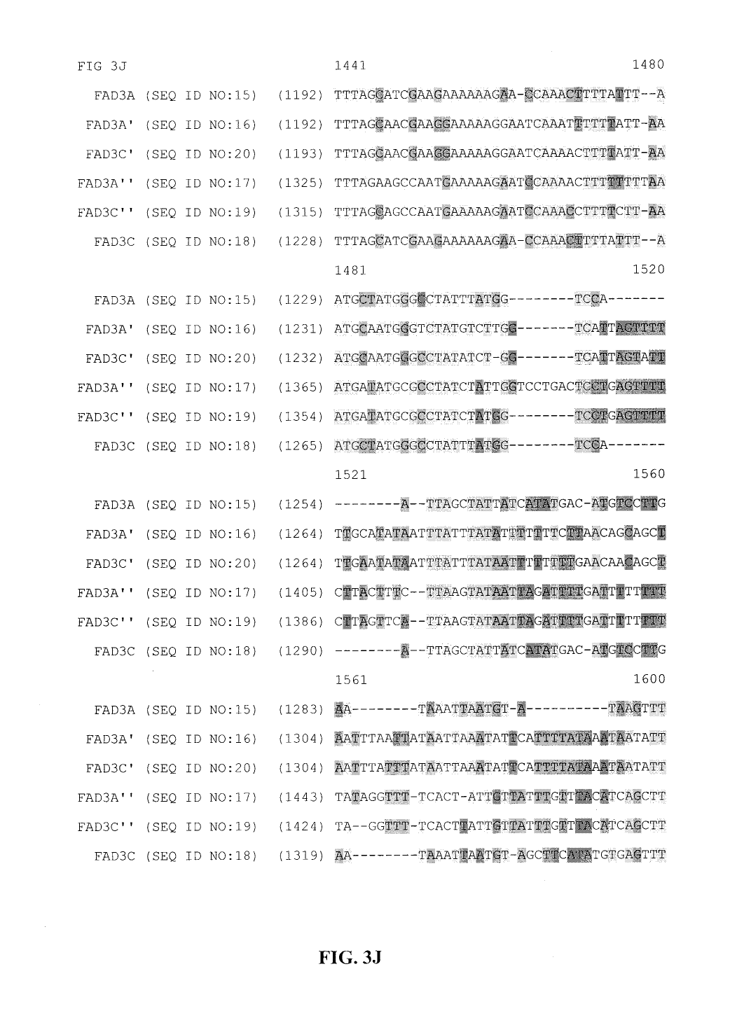

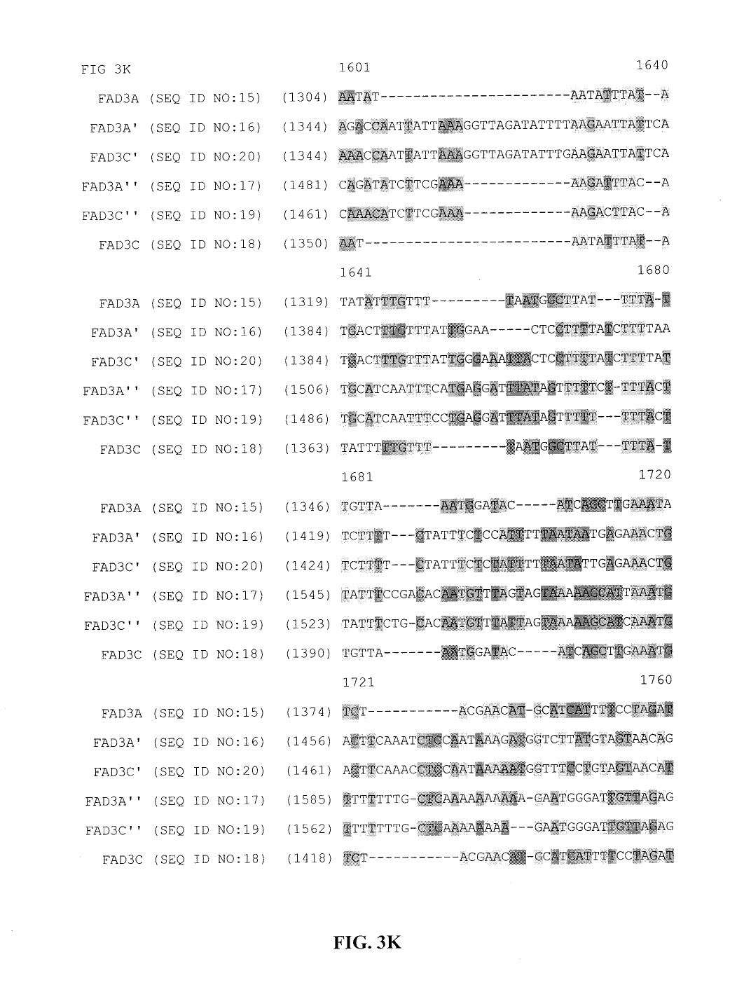

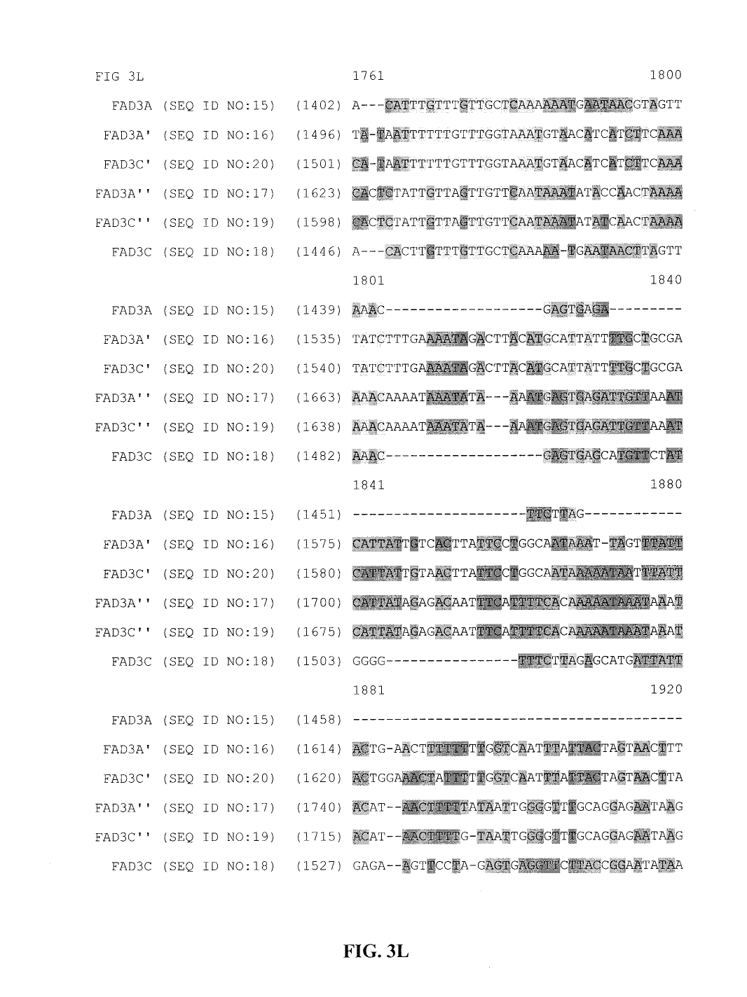

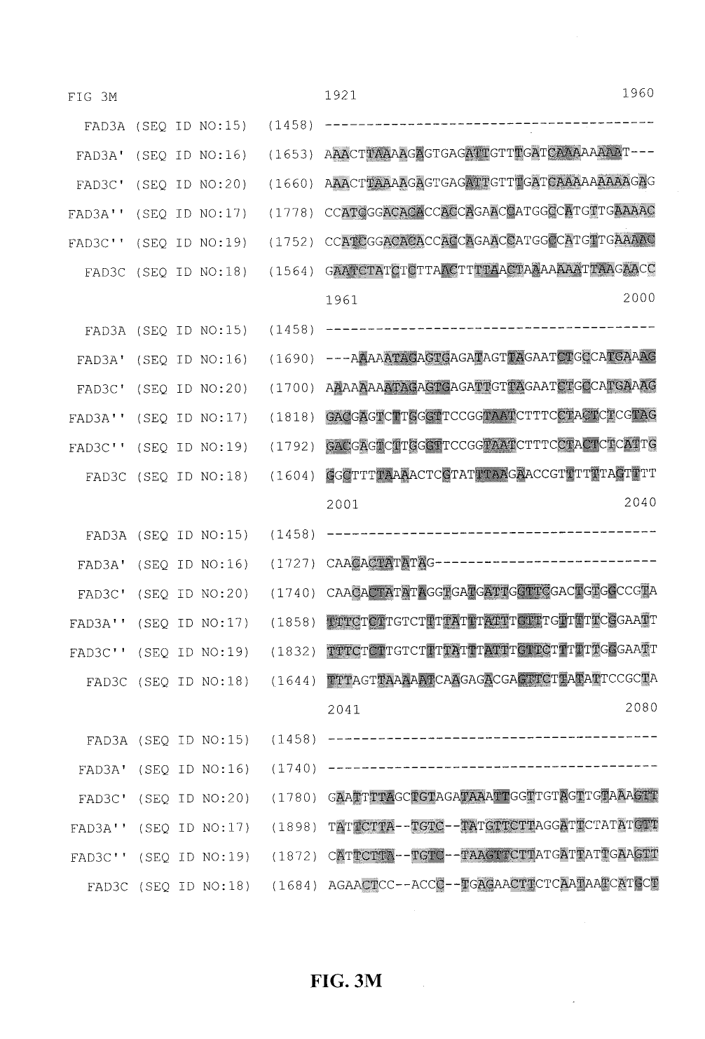

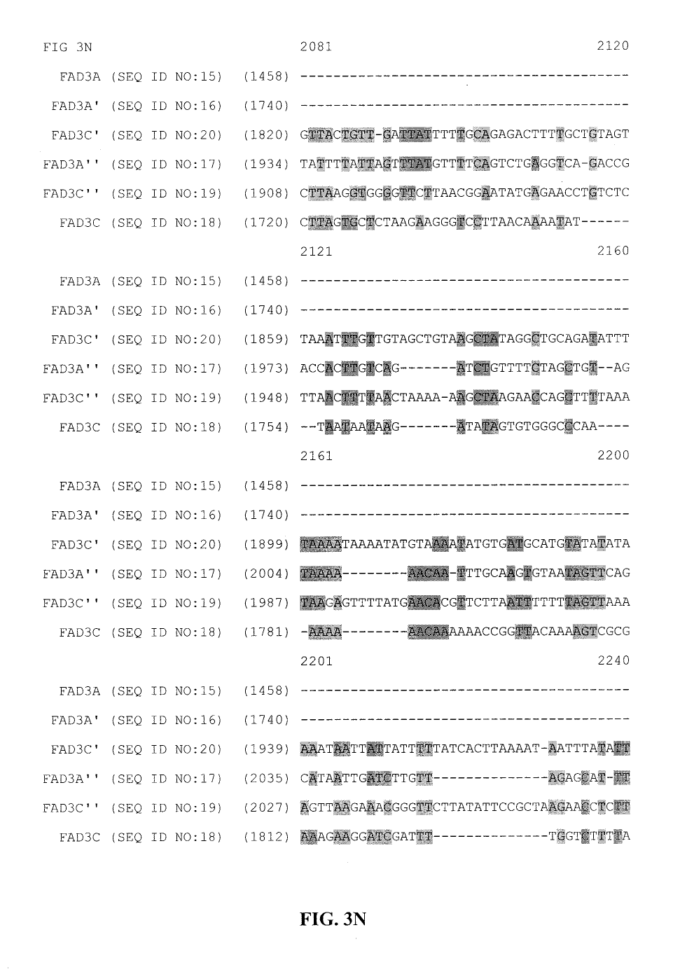

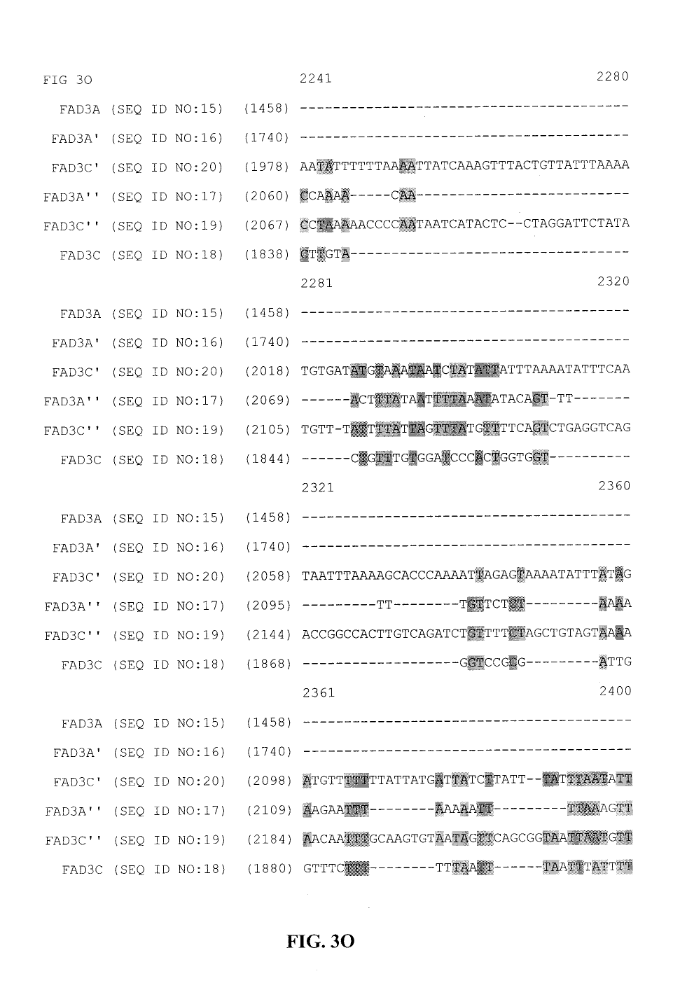

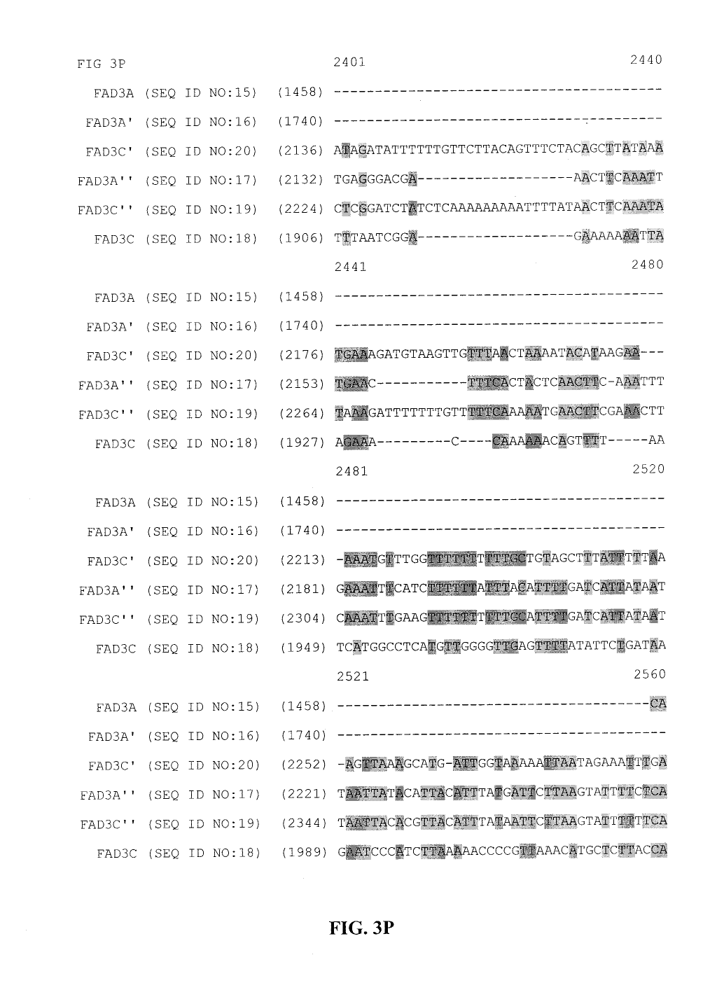

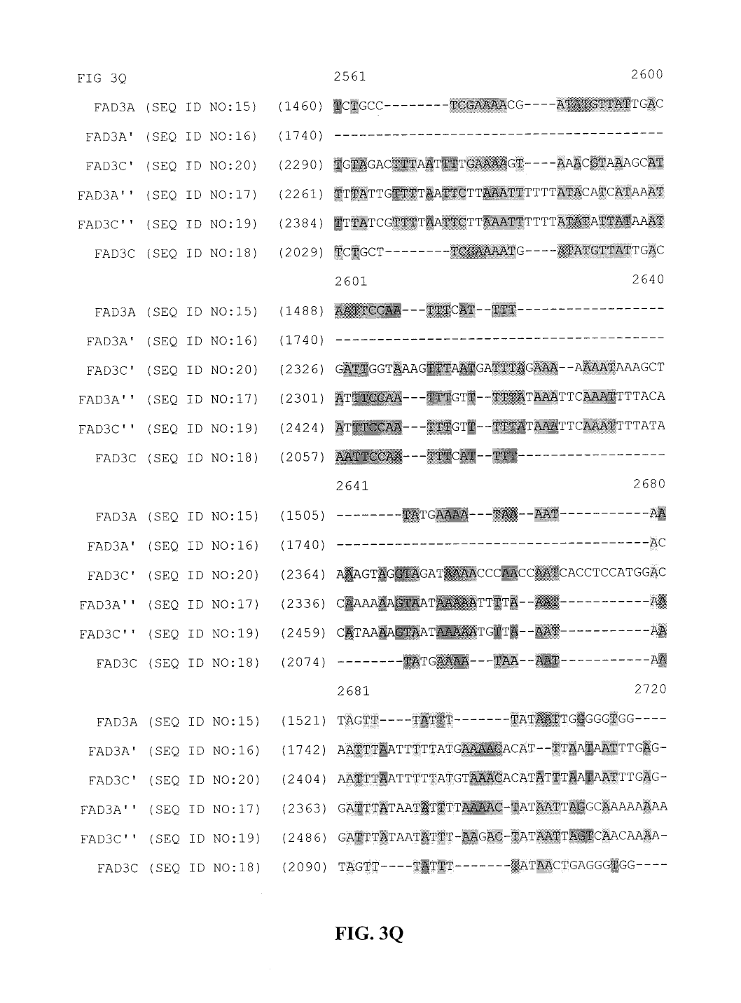

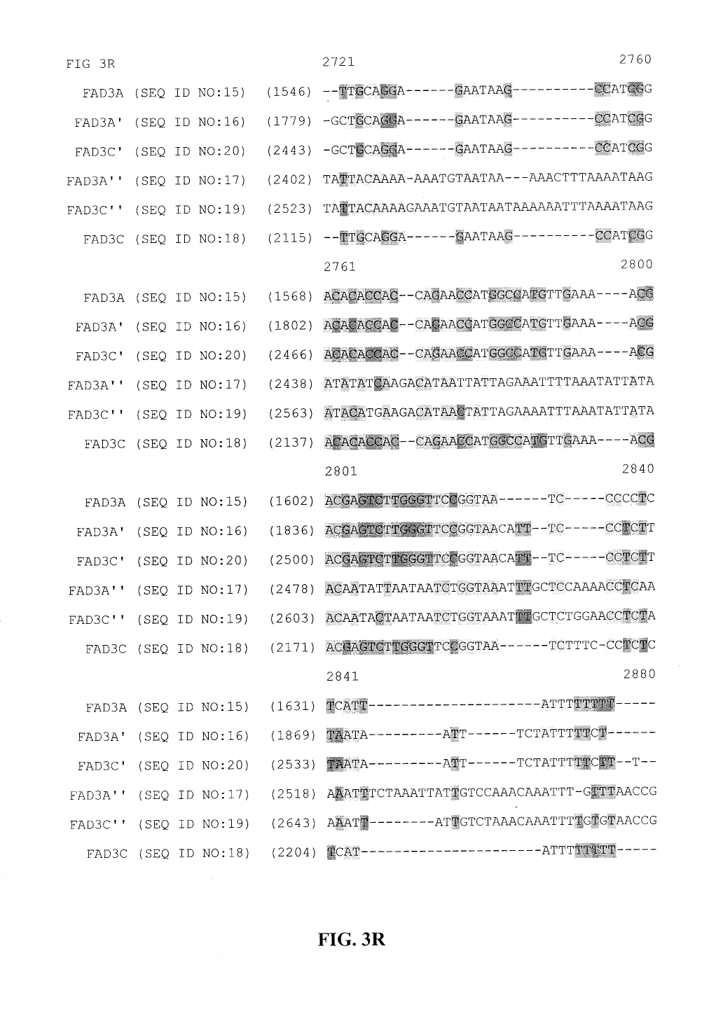

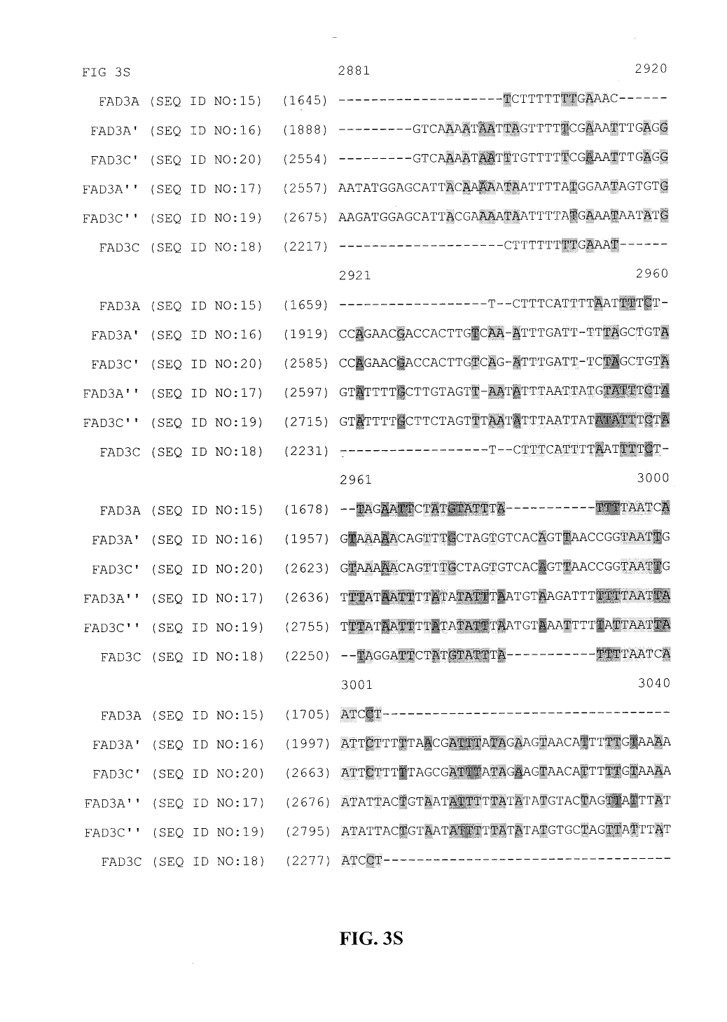

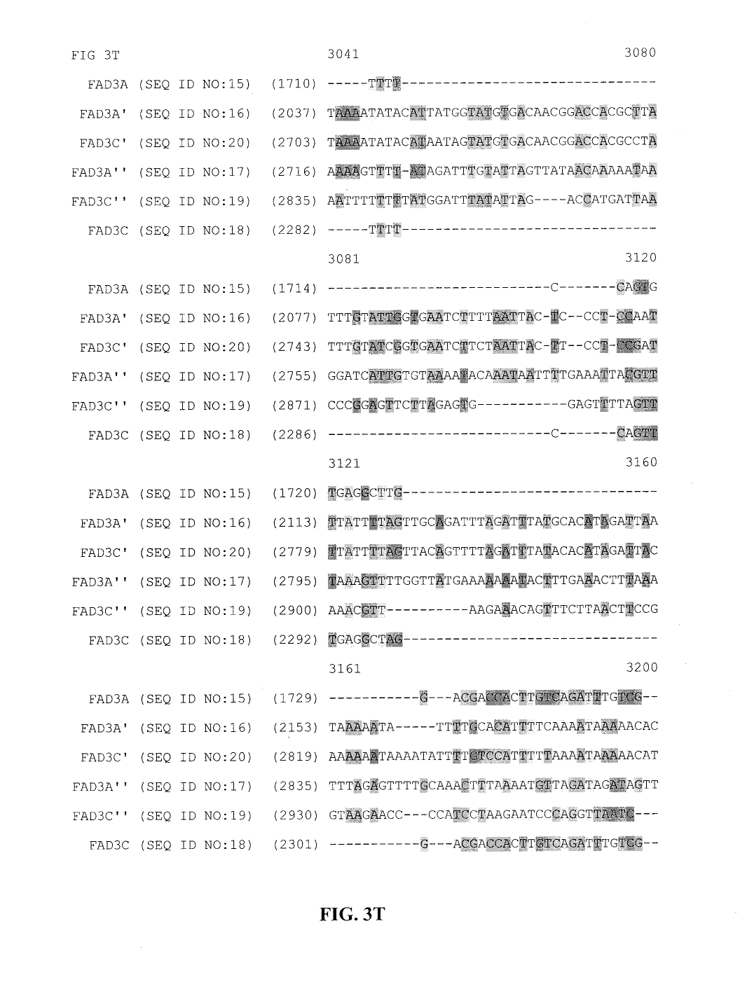

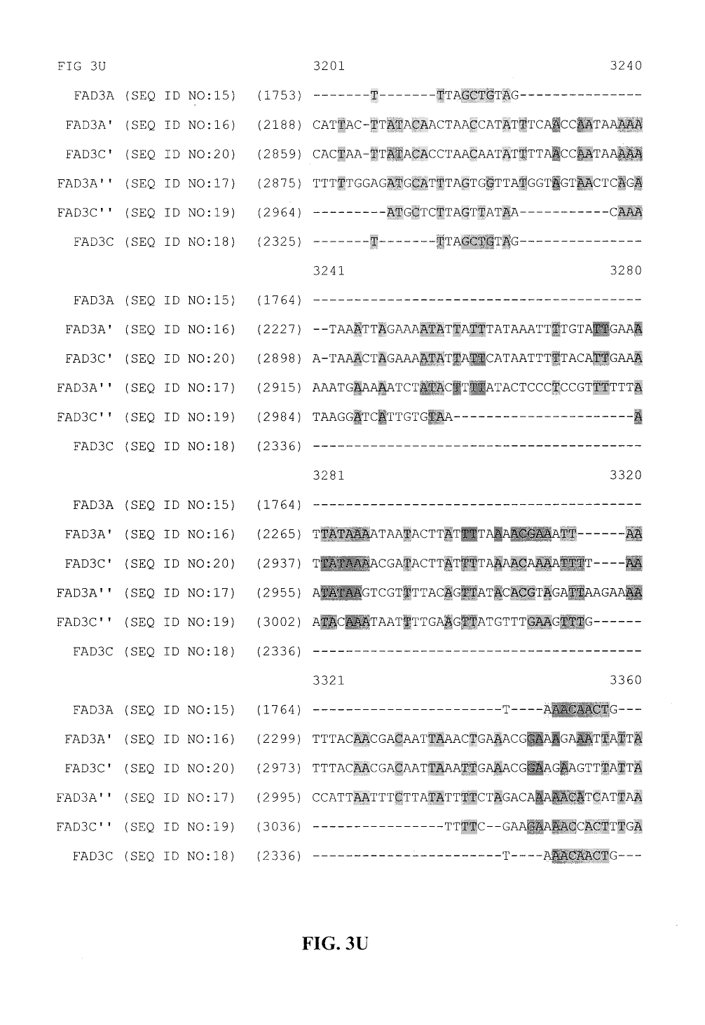

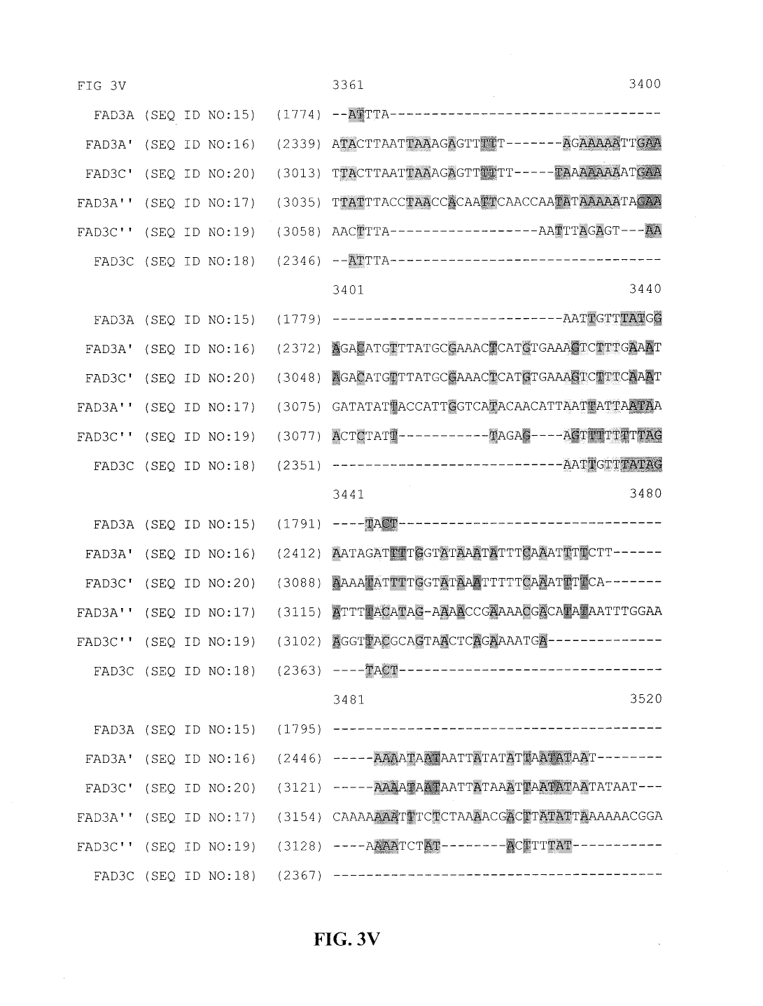

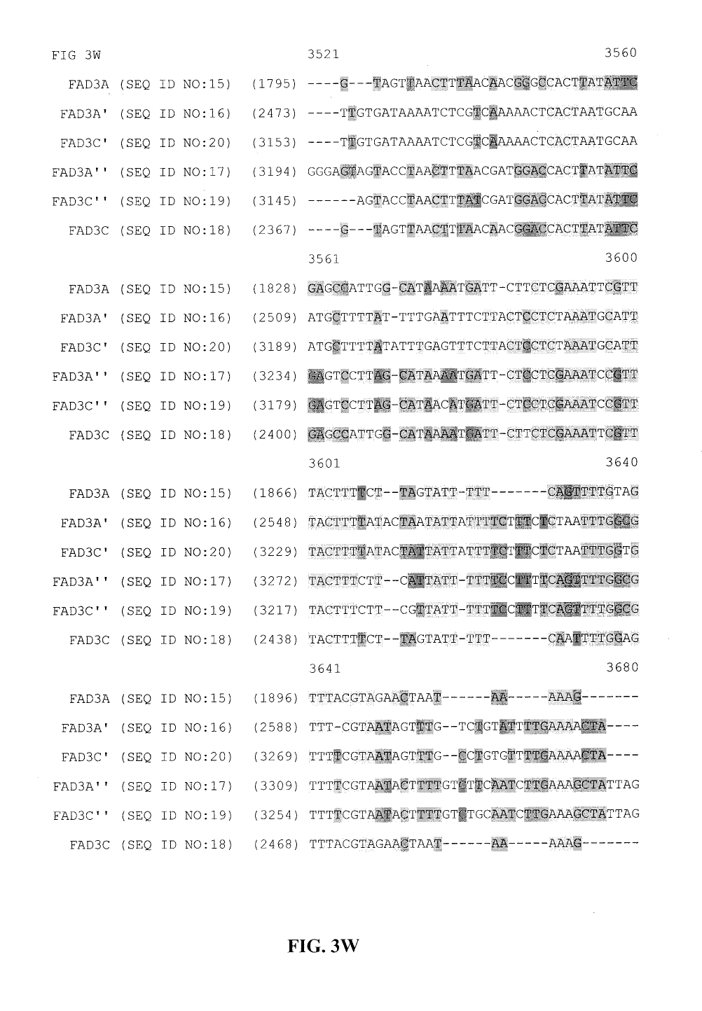

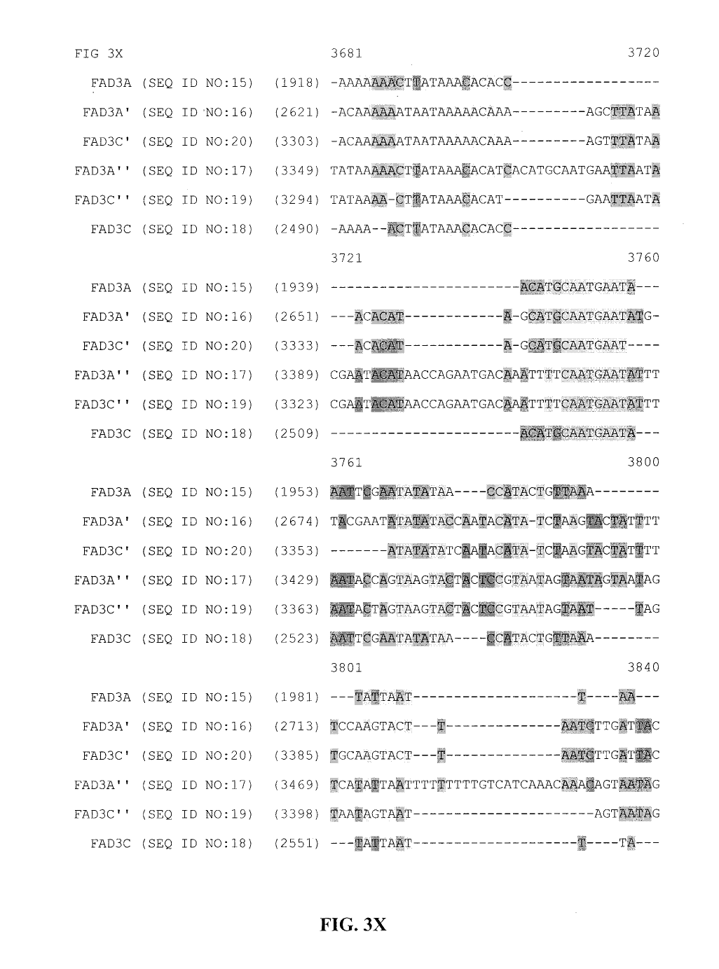

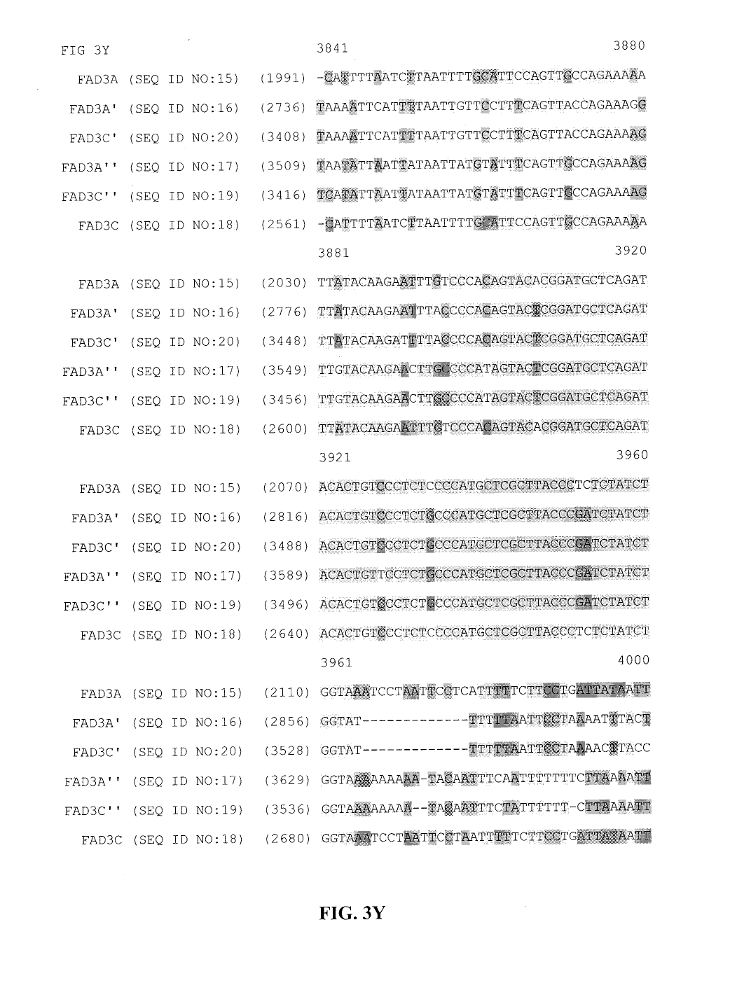

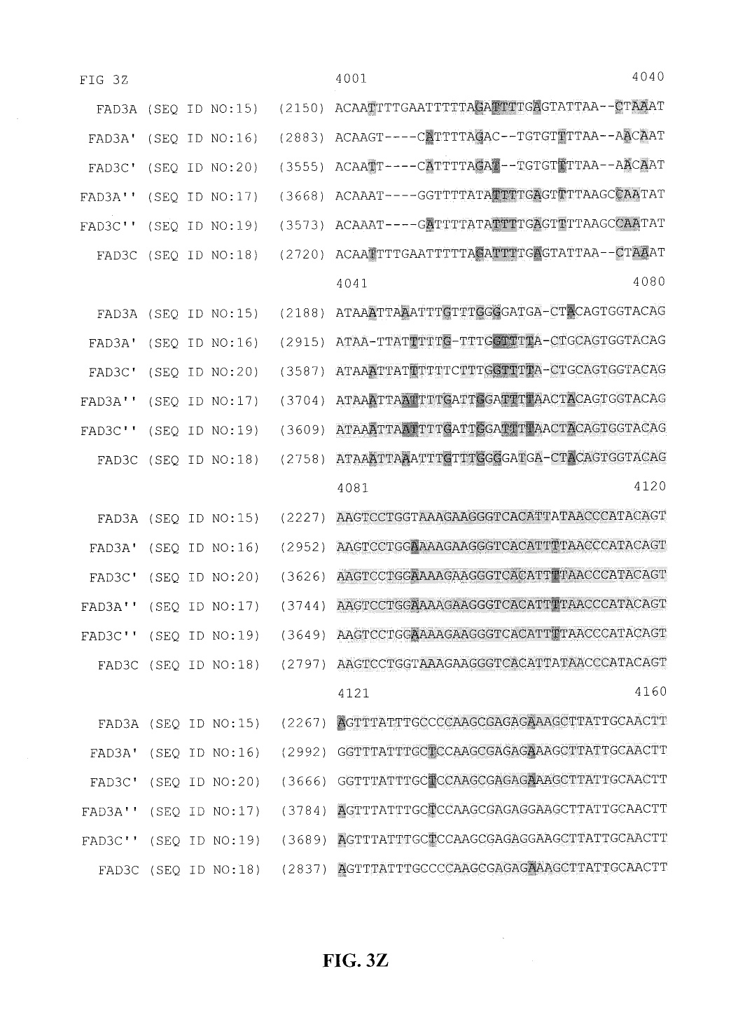

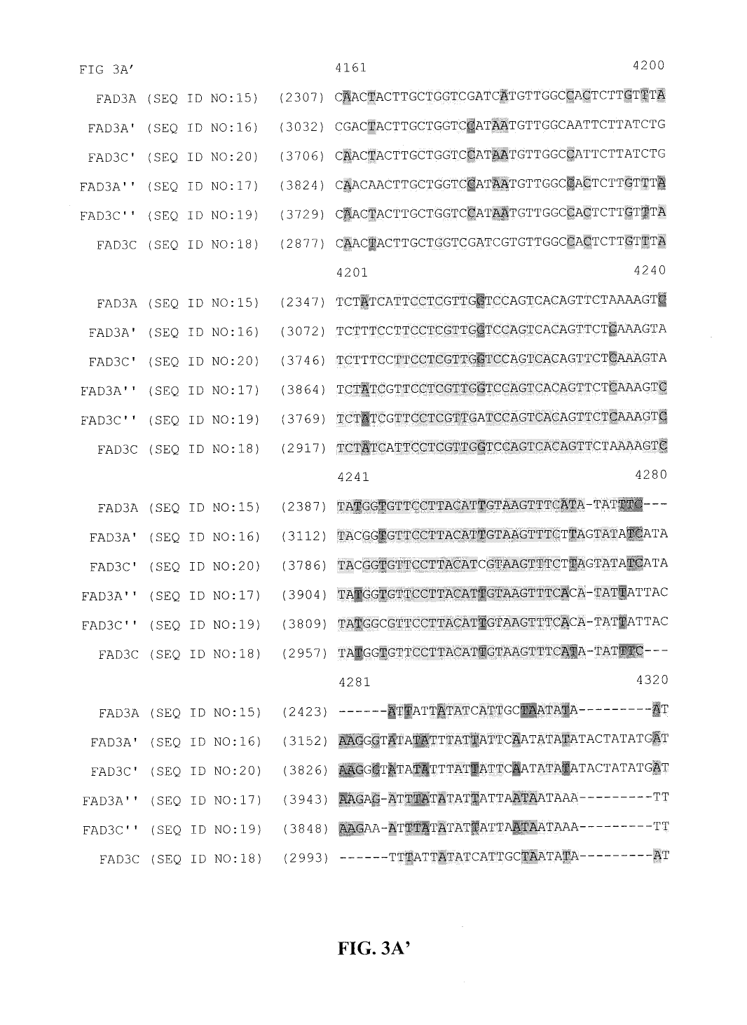

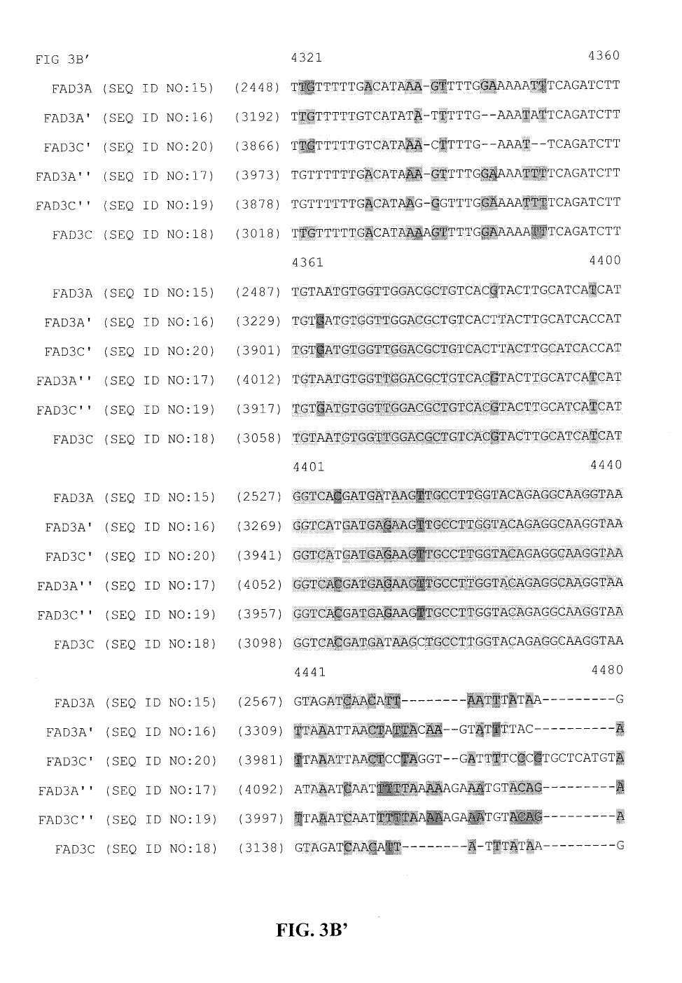

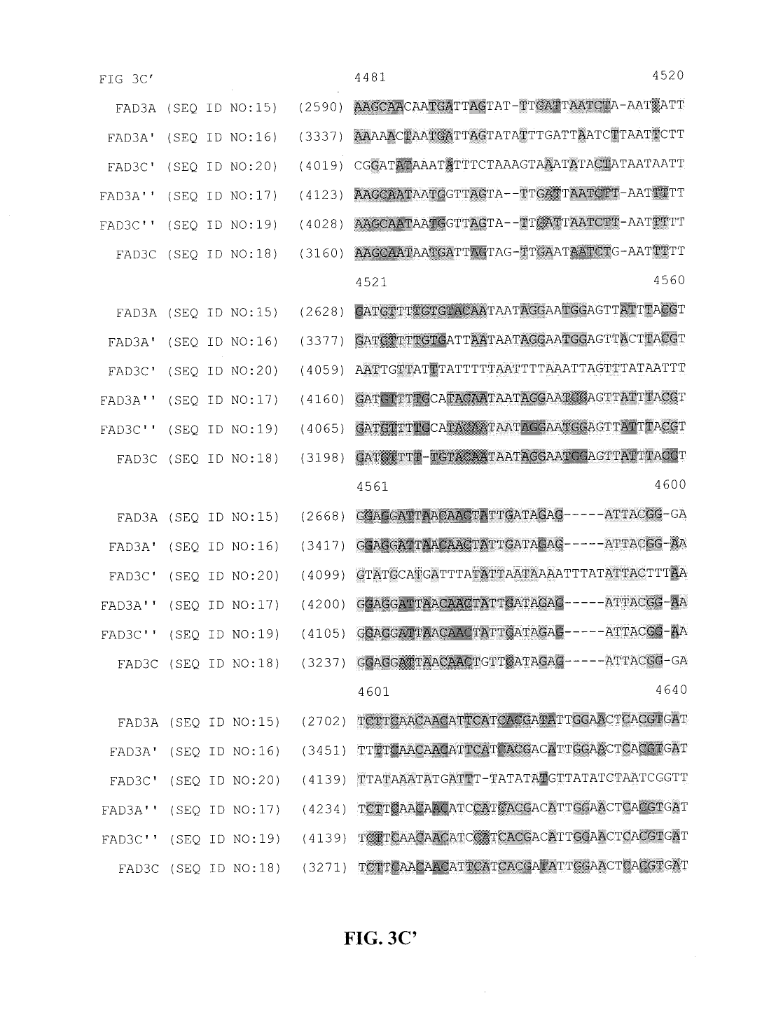

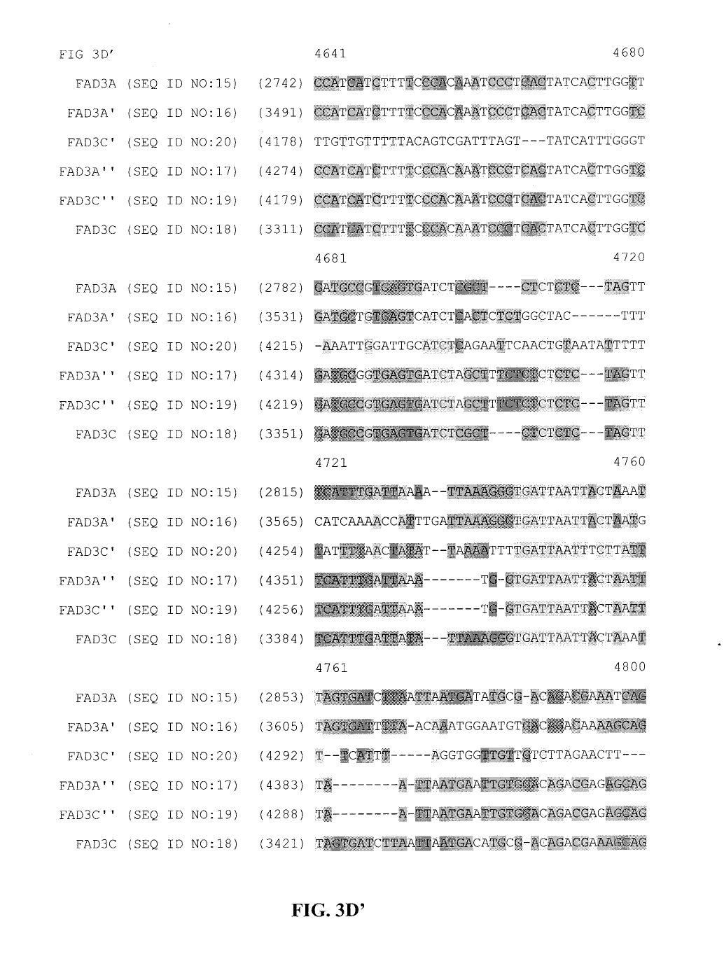

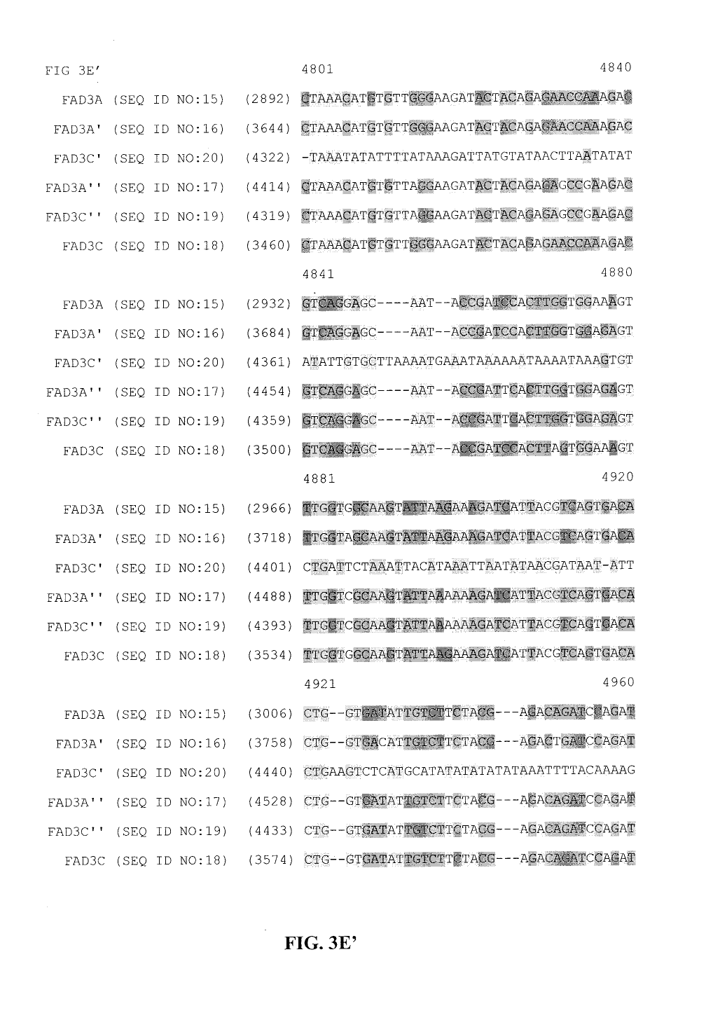

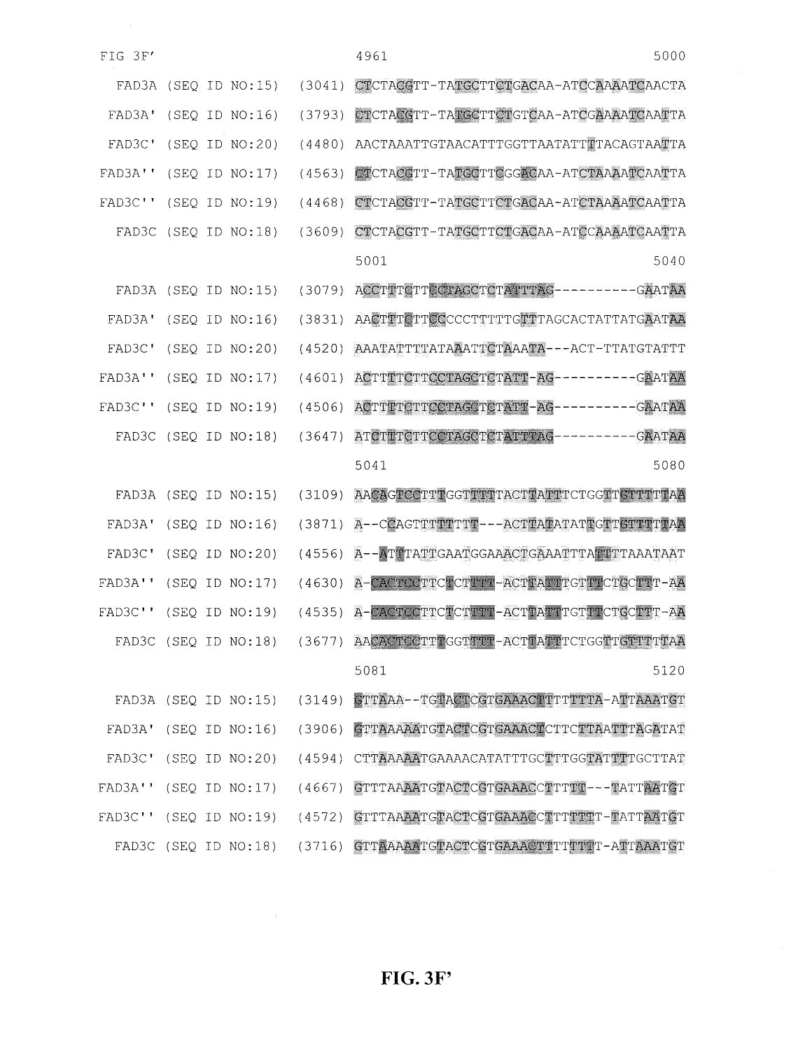

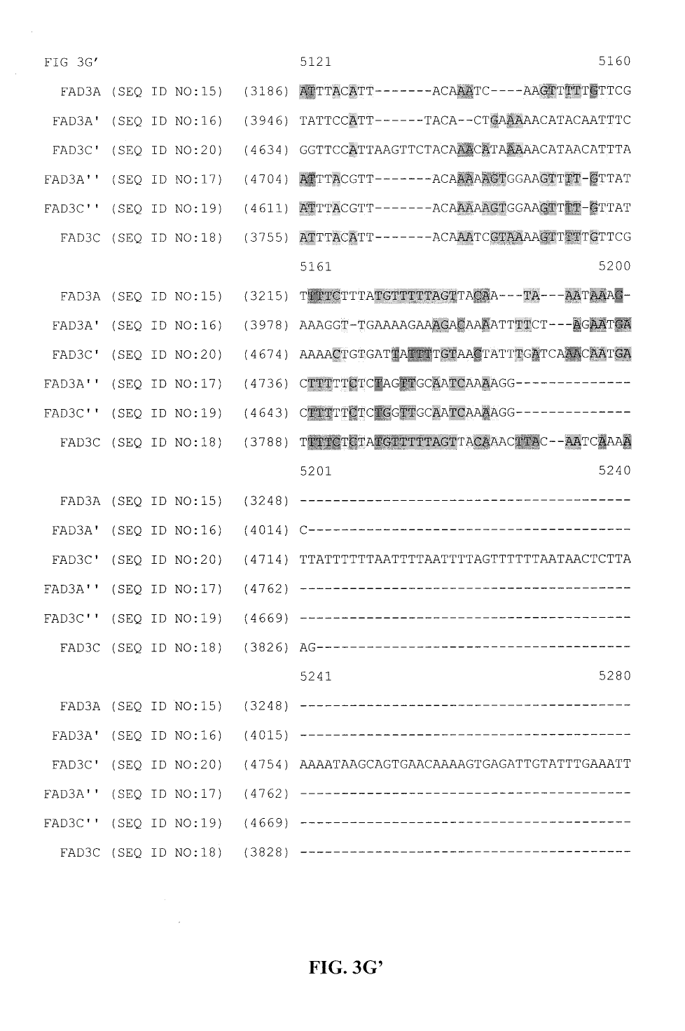

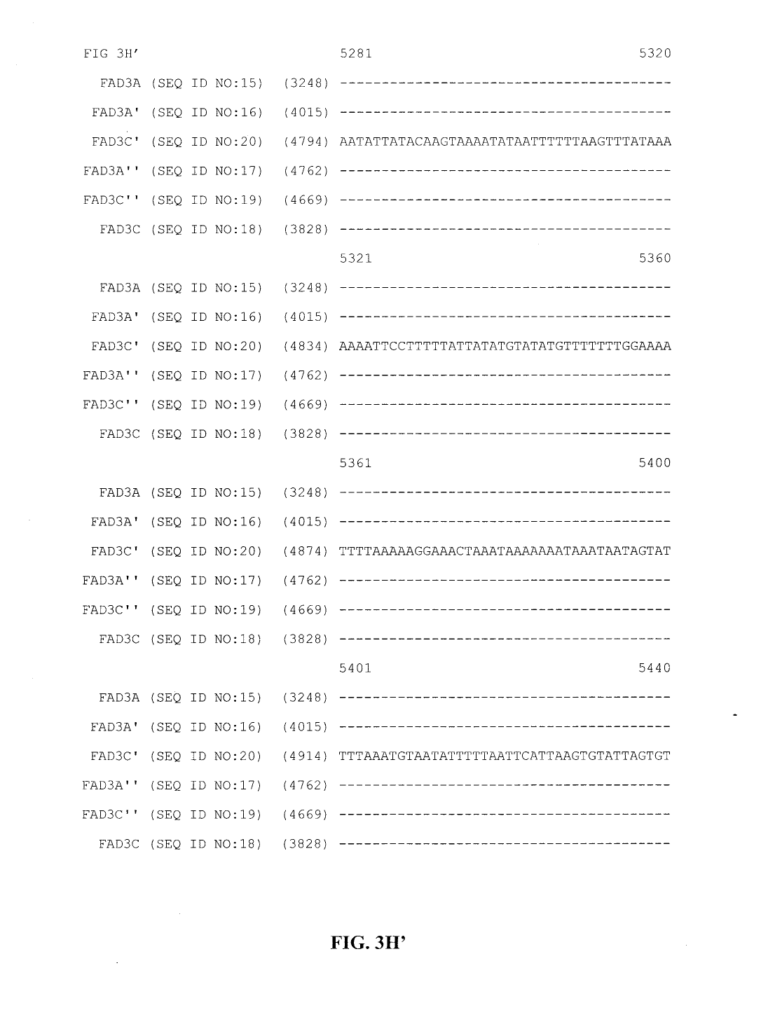

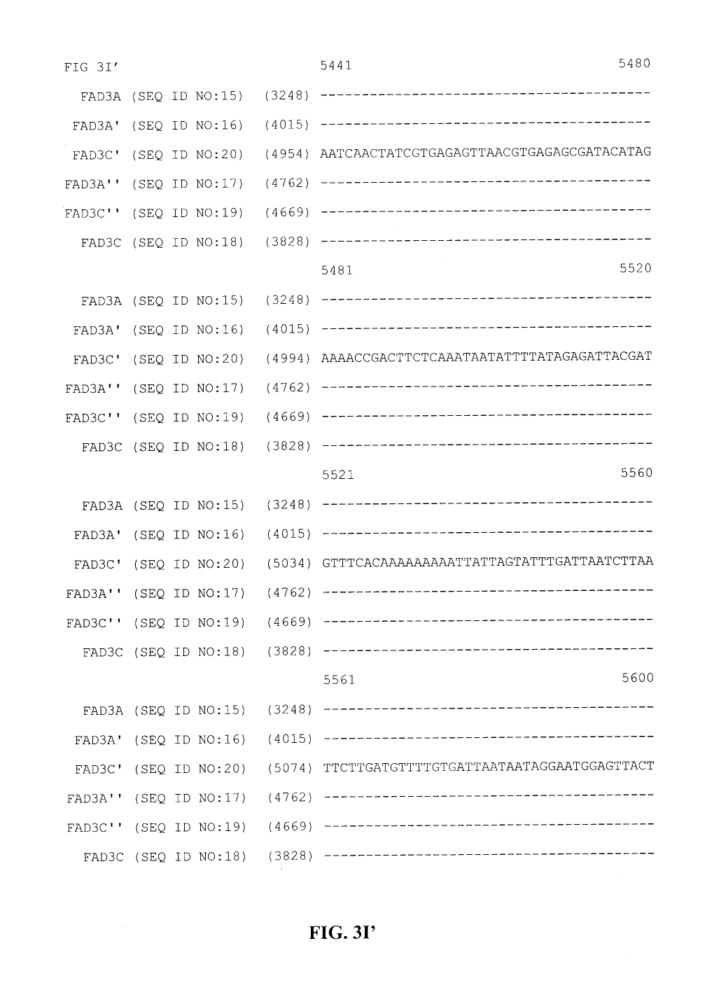

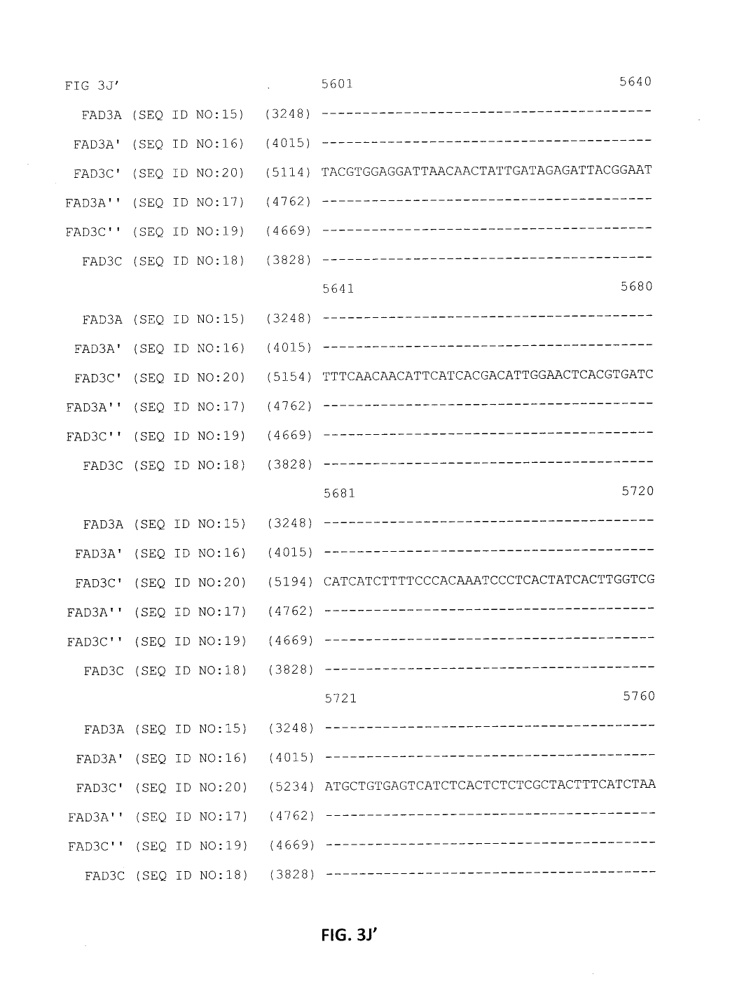

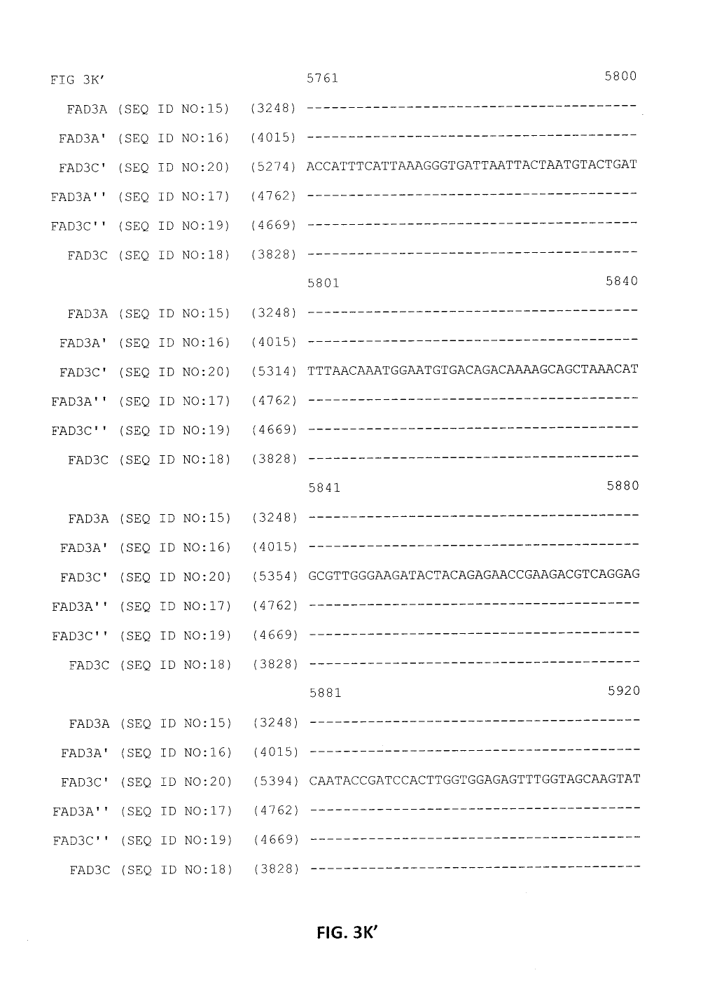

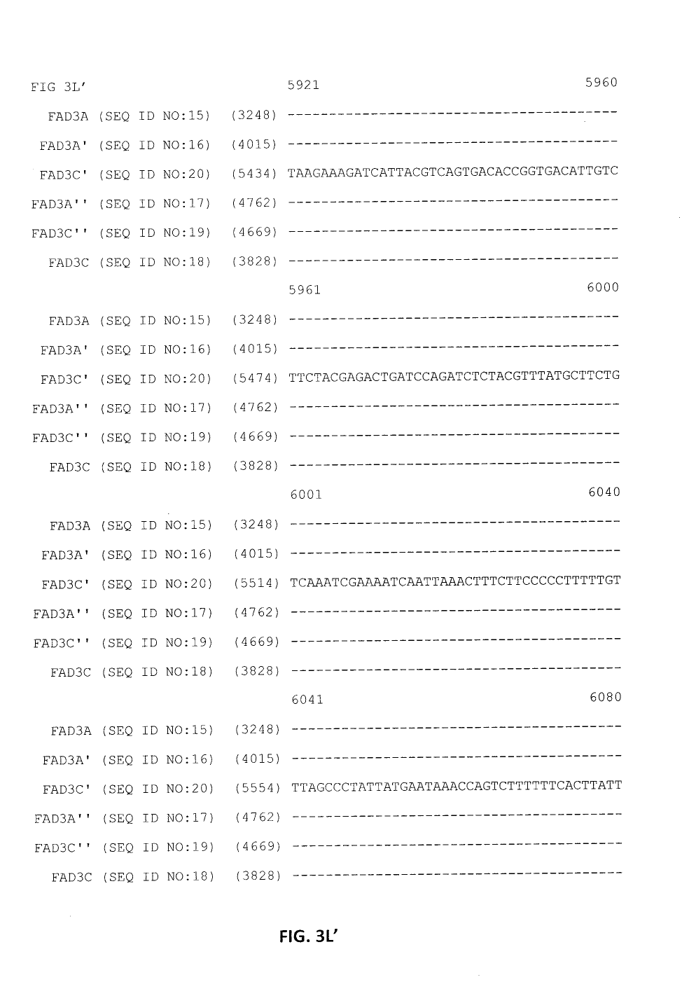

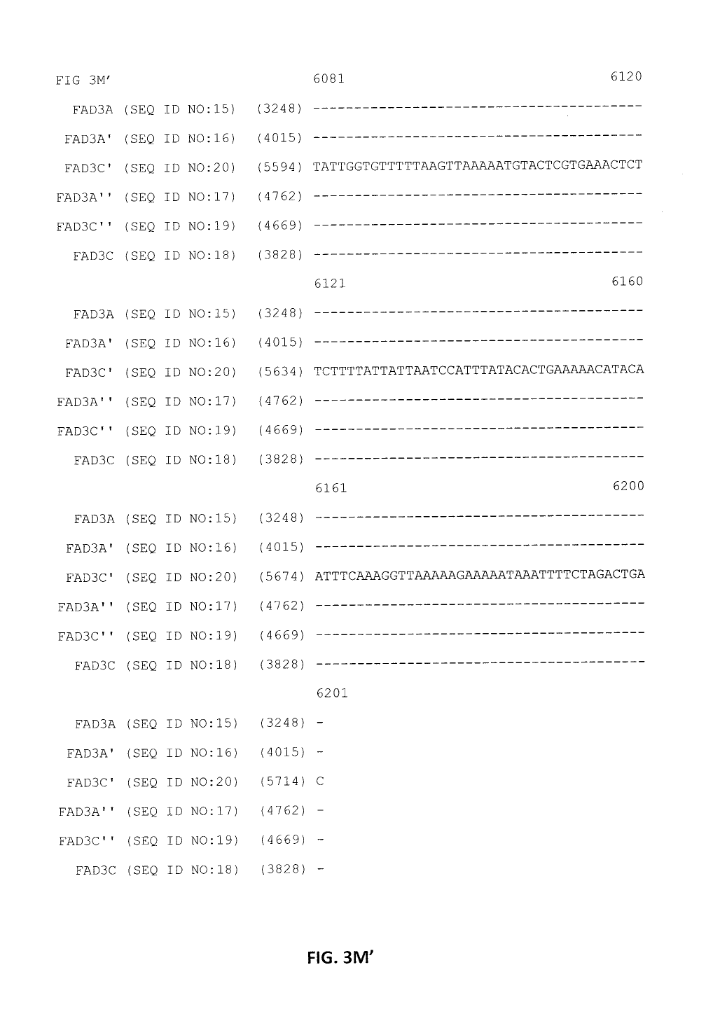

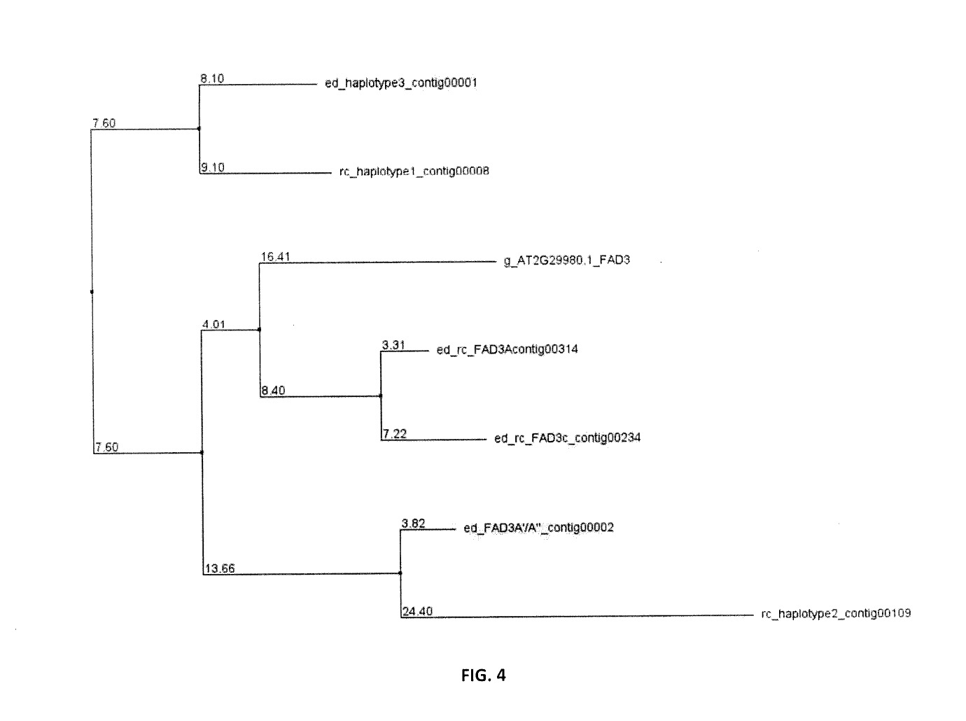

Sequence analysis of the six FAD3 genes was conducted using a sequence alignment program and a neighbor-joining tree using percentage of identity. The sequence alignment was made via the ALIGNX® program from the Vector NTI Advance 11.0 computer program (Life Technologies, Carlsbad, Calif.) and is shown in FIG. 3. ALIGNX® uses a modified Clustal W algorithm to generate multiple sequence alignments of either protein or nucleic acid sequences for similarity comparisons and for annotation. The neighbor-joining tree was created with JALVIEWv2.3® software and is shown in FIG. 4. (Waterhouse et al. (2009) Bioinformatics 25 (9) 1189-1191). The contigs identified as containing FAD3 genes were used as BLASTn queries against a database of Arabidopsis thaliana genes. The region of each of the 6 contigs containing the FAD3 gene was identified through comparison to the Arabidopsis thaliana FAD3 gene (Genbank Accession No: At2g29980). The FAD3 contigs were then orientated such that all FAD3 genes were in the 5′ to 3′ orientation. FAD3 contigs were trimmed to contain as many as 2 upstream (5′) and 1 downstream (3′) Arabidopsis thaliana genes where possible. Once orientated the complete coding region of the FAD3 genes were extracted from each contig and used to generate a Neighbour joining tree to display the relationship between the different FAD3 gene family members. The 6 FAD3 family members were aligned into 3 pairs of FAD3 genes (FIG. 4).

PCR Based Screening

A cohort of PCR primers were design to screen the aforementioned BAC library. The primers were designed as either universal primers, which would amplify all members of the gene family, or as gene specific primers for targeted allele amplification. The PCR primers were designed to be 20 bp long (+/−1 bp) and contain a G/C content of 50% (+/−8%). Table 2 and Table 3 lists the primers which were designed and synthesized. The clones of the BAC library were pooled and screened via the Polymerase Chain Reaction (PCR).

TABLE 2
Primer sequences used for PCR amplification
of FAD3 sequences.

| Primer Name: | SEQ ID NO: | Sequence: |
|---|---|---|
| D_uni_F3_F1 | SEQ ID NO: 21 | GAATAAGCCATCGGACACAC |
| D_spec_F3_F2 | SEQ ID NO: 22 | ATGCGAACGGAGACGAAAGG |
| D_spec_F3_F3 | SEQ ID NO: 23 | TGTTAACGGAGATTCCGGTG |
| D_spec_F3_F4 | SEQ ID NO: 24 | GTAGCAATGTGAACGGAGAT |
| D_uni_F3_R1 | SEQ ID NO: 25 | CAGTGTATCTGAGCATCCG |
| D_spec_F3_R2 | SEQ ID NO: 26 | GTGGCCGAGTACGAAGATAG |
| D_spec_F3_R3 | SEQ ID NO: 27 | CAGTAGAGTGGCCAGAGGA |

TABLE 3
PCR primer sequences designed for BAC
library screening for FAD2 gene
identification.

| Primer Name | SEQ ID NO: | Sequence |
|---|---|---|
| D_UnivF2_F1 | SEQ ID NO: 28 | ATGGGTGCAGGTGGAAGAATG |
| D_UnivF2_F2 | SEQ ID NO: 29 | AGCGTCTCCAGATATACATC |
| D_UnivF2_R1 | SEQ ID NO: 30 | ATGTATATCTGGAGACGCTC |
| D_UnivF2_R2 | SEQ ID NO: 31 | TAGATACACTCCTTCGCCTC |
| D_SpecificF2_ | SEQ ID NO: 32 | TCTTTCTCCTACCTCATCTG |
| F3 | | |
| D_SpecificF2_ | SEQ ID NO: 33 | TTCGTAGCTTCCATCGCGTG |
| R3 | | |
| D_UnivF2_F4 | SEQ ID NO: 34 | GACGCCACCATTCCAACAC |
| D_UnivF2_R4 | SEQ ID NO: 35 | ACTTGCCGTACCACTTGATG |

A Two different sets of conditions were used for the polymerase chain reactions (PCR). The first series of PCR reactions contained: 1×PCR buffer (containing dNTPs); 1.5 mM MgCl₂; 200 μM of 0.25 U IMMOLASE® DNA polymerase (Bioline, London, UK); 250 nM of each primer; and, about 5-10 ng template DNA. A second series of PCR reactions were developed for the amplification of genomic DNA and contained: 5-10 ng of genomic DNA, 1×PCR buffer, 2 mM dNTPs, 0.4 μM forward and reverse primer, and 0.25 U IMMOLASE® DNA polymerase (Bioline, London, UK). Amplifications were pooled into a final volume of 13 μL and amplified using an MJ PTC200® thermocycler (BioRad, Hercules, Calif.) or an ABI 9700 GENE AMP SYSTEM® (Life Technologies, Carlsbad, Calif.). PCR based screening of specific plates was conducted using a 4 dimension screening approach based on the screening system described by Bryan et al (Scottish Crops Research Institute annual report: 2001-2002) with the above described PCR conditions. Following PCR based screening of pooled BAC libraries; the amplified PCR product was sequenced using a direct Sanger sequencing method. The amplified products were purified with ethanol, sodium acetate and EDTA following the BIGDYE® v3.1 protocol (Applied Biosystems) and electrophoresis was performed on an ABI3730xl® automated capillary electrophoresis platform.

Following PCR based screening and conformational Sanger sequencing, a collection of plates were identified that contained the various different FAD2 and FAD3 gene family members. A total of four unique FAD2 and FAD3 paralogous gene sequences were identified (Table 4 and Table 5). A total of two plates per each FAD2 and FAD3 paralogous gene sequence were chosen to undergo plate screening to identify the specific well and clone within the plate that contained the FAD2 and FAD3 gene (Table 4 and Table 5). The specific wells were identified for both of the plates and an individual clone was selected for each of the FAD2 and FAD3 gene family members.

TABLE 4
Identification of the BAC clone plates that provided
positive reaction with the detailed PCR primer combinations,
along with two plate identities that were taken forward
for clone identification within the plate.

| Gene | | Positive Plate | Chosen | Well |
|---|---|---|---|---|
| Name | Primer Sets | Pools | Plates | Id |
| FAD2A | F4 + R1, | 8, 27, 30, 83, 109, | Plate 199 | L23 |
| | F1 + R1, | 147, 180, 199, 209, | Plate 27 | D20 |
| | F1 + R4, | 251, 288 | | |
| | F3 + R3 | | | |
| FAD2-1 | F1 + R4, | 12, 89, 123, 148, | Plate 123 | N17 |
| | F4 + R1, | 269 | Plate 148 | B15 |
| | F1 + R1, | | | |
| | F2 + R2 | | | |
| FAD2-2 | F4 + R1, | 24, 44, 46, 47, 80, | Plate 44 | H03 |
| | F1 + R1, | 91, 104, 110, 119, | Plate 121 | A17 |
| | F1 + R4, | 121, 124, 248 | | |
| | F2 + R2 | | | |
| FAD2-3 | F1 + R4, | 8, 62, 113, 205, 276 | Plate 62 | I16 |
| | F4 + R1, | | Plate 205 | K11 |
| | F1 + R1, | | | |
| | F3 + R3 | | | |

TABLE 5
Identification of the BAC clone plates that provided
positive reaction with the detailed PCR primer combinations,
along with two plate identities that were taken forward
for clone identification within the plate.

| | | Positive Plate | Chosen |
|---|---|---|---|
| Gene Name | Primer Sets | Pools | Plates |
| FAD3A | F2 + R2 | 16, 231 | Plate 16 |
| (FAD3A-1) | | | Plate 231 |
| FAD3C | F4 + R2 | 18, 27, 136, 178, | Plate 18 |
| | | 211,232 | Plate 27 |
| FAD3C″ | F4 + R2, | 23, 44, 53, 56, 77, | Plate 44 |
| (Haplotype1) | F4 + R3, | 116, 158, 199, 209, | Plate 199 |
| | F3 + R3 | 278, 280, 282, 283, | |
| | | 284, 286 | |
| FAD3A′ | F4 + R2 | 52, 121, 139 | Plate 121 |
| (FAD3A′/ | | | Plate 139 |
| FAD3A″) | | | |
| FAD3C′ | F4 + R2 | 144, 188, 235 | Plate 144 |
| (Haplotype2) | | | Plate 188 |
| FAD3A″ | F4 + R3 and | 69, 105, 106, 229, | Plate 69 |
| (Haplotype3) | F3 + R3 | 242, 247, 248 | Plate 106 |

The single BAC clone, for each identified FAD gene family member, was further analysed via sequencing. The DNA was isolated for the BAC clone and was prepared for sequencing using a LARGE CONSTRUCT KIT® (Qiagen, Valencia, Calif.) following the manufacturer's instructions. The extracted BAC DNA was prepared for sequencing using GS-FLX® Titanium Technology (Roche, Indianapolis, Ind.) following manufacturer's instructions. Sequencing reactions were performed using a physically sectored GS-FLX TI® Pico-titer plate with the BACs pooled in pairs for optimal data output. The BACs were combined in pairs where the FAD2 gene was paired with a FAD3 gene. All generated sequence data was assembled by NEWBLER v2.0.01.14® (454 Life Sciences, Branford, Conn.). The assembled contigs were manually assessed for the presence of the corresponding FAD gene using SEQUENCHERv3.7® (GeneCodes, Ann Arbor, Mich.).

After the full genomic sequence of all four FAD2 and six FAD3 genes had been identified and fully characterized, zinc finger nucleases were designed to bind to the sequences for each specific gene family member.

### Example 2

### Design of Zinc Finger Binding Domains Specific to Fad2 Genes

Zinc finger proteins directed against DNA sequences encoding various functional sequences of the FAD2 gene locus were designed as previously described. See, e.g., Urnov et al. (2005) Nature 435:646-651. Exemplary target sequence and recognition helices are shown in Table 6 and Table 8 (recognition helix regions designs) and Table 7 and Table 9 (target sites). In Table 8 and Table 9, nucleotides in the target site that are contacted by the ZFP recognition helices are indicated in uppercase letters; non-contacted nucleotides indicated in lowercase. Zinc Finger Nuclease (ZFN) target sites were designed to bind five target sites of FAD2A, and seven target sites of FAD3. The FAD2 and FAD3 zinc finger designs were incorporated into zinc finger expression vectors encoding a protein having at least one finger with a CCHC structure. See, U.S. Patent Publication No. 2008/0182332. In particular, the last finger in each protein had a CCHC backbone for the recognition helix. The non-canonical zinc finger-encoding sequences were fused to the nuclease domain of the type IIS restriction enzyme FokI (amino acids 384-579 of the sequence of Wah et al., (1998) Proc. Natl. Acad. Sci. USA 95:10564-10569) via a four amino acid ZC linker and an opaque-2 nuclear localization signal derived from Zea mays to form FAD2A zinc-finger nucleases (ZFNs). Expression of the fusion proteins was driven by a relatively strong constitutive promoter such as a promoter derived from the Cassaya Vein Mosaic Virus (CsVMV) promoter and flanked by the Agrobacterium tumefaciens ORF23 3′ UnTranslated Region (AtuORF23 3′UTR v1). The self-hydrolyzing 2A encoding nucleotide sequence from Thosea asigna virus (Szymczak et al., 2004) was added between the two Zinc Finger Nuclease fusion proteins that were cloned into the construct. Exemplary vectors are described below.

The optimal zinc fingers were verified for cleavage activity using a budding yeast based system previously shown to identify active nucleases. See, e.g., U.S. Patent Publication No. 20090111119; Doyon et al. (2008) Nat. Biotechnol. 26:702-708; Geurts et al. (2009) Science 325:433. Zinc fingers for the various functional domains were selected for in-vivo use. Of the numerous ZFNs that were designed, produced and tested to bind to the putative FAD genomic polynucleotide target sites, a ZFNs were identified as having in vivo activity at high levels, and selected for further experimentation. These ZFNs were characterized as being capable of efficiently binding and cleaving the unique FAD2 genomic polynucleotide target sites in planta.

TABLE 6
FAD3 Zinc Finger Designs

| ZFP | F1 | F2 | F3 | F4 | F5 | F6 |
|---|---|---|---|---|---|---|
| 27961 | RSDNLAR | QKKDRSY | RSDNLAR | QRGNRNT | RSDHLSR | RNQDRTN |
| | (SEQ ID | (SEQ ID | (SEQ ID | (SEQ ID | (SEQ ID | (SEQ ID |
| | NO: 178) | NO: 179) | NO: 180) | NO: 181) | NO: 182) | NO: 183) |
| 27962 | DRSNLSR | RQDSRSQ | QSSDLSR | DRSALAR | TSGSLTR | N/A |
| | (SEQ ID | (SEQ ID | (SEQ ID | (SEQ ID | (SEQ ID | |
| | NO: 184) | NO: 185) | NO: 186) | NO: 187) | NO: 188) | |
| 27973 | QSSDLSR | AASNRSK | TSGSLSR | RSDALAR | RSDVLST | WGRLRKL |
| | (SEQ ID | (SEQ ID | (SEQ ID | (SEQ ID | (SEQ ID | (SEQ ID |
| | NO: 189) | NO: 190) | NO: 191) | NO: 192) | NO: 193) | NO: 194) |
| 27974 | ERGTLAR | RSDDLTR | RSDHLSA | QHGALQT | TSGNLTR | QSGHLSR |
| | (SEQ ID | (SEQ ID | (SEQ ID | (SEQ ID | (SEQ ID | (SEQ ID |
| | NO: 195) | NO: 196) | NO: 197) | NO: 198) | NO: 199) | NO: 200) |
| 27987 | TSGSLTR | RSDHLSQ | CTRNRWR | RSDNLSE | ASKTRKN | N/A |
| | (SEQ ID | (SEQ ID | (SEQ ID | (SEQ ID | (SEQ ID | |
| | NO: 201) | NO: 202) | NO: 203) | NO: 204) | NO: 205) | |
| 27990 | TSGSLSR | TSSNRAV | TSGNLTR | DRSALAR | RSDVLSE | RNFSLTM |
| | (SEQ ID | (SEQ ID | (SEQ ID | (SEQ ID | (SEQ ID | (SEQ ID |
| | NO: 206) | NO: 207) | NO: 208) | NO: 209) | NO: 210) | NO: 211) |
| 27991 | QSGDLTR | TSGSLSR | QSGNLAR | TSGSLSR | QSGSLTR | N/A |
| | (SEQ ID | (SEQ ID | (SEQ ID | (SEQ ID | (SEQ ID | |
| | NO: 212) | NO: 213) | NO: 214) | NO: 215) | NO: 216) | |
| 27992 | DRSHLAR | TSGSLSR | TSSNRAV | TSGNLTR | DRSALAR | N/A |
| | (SEQ ID | (SEQ ID | (SEQ ID | (SEQ ID | (SEQ ID | |
| | NO: 217) | NO: 218) | NO: 219) | NO: 220) | NO: 221) | |
| 28004 | QSGNLAR | HLGNLKT | RSDHLSQ | TARLLKL | QSGNLAR | QTSHLPQ |
| | (SEQ ID | (SEQ ID | (SEQ ID | (SEQ ID | (SEQ ID | (SEQ ID |
| | NO: 222) | NO: 223) | NO: 224) | NO: 225) | NO: 226) | NO: 227) |
| 28005 | RSDNLSV | TSGHLSR | TSGSLTR | RSDALST | DRSTRTK | N/A |
| | (SEQ ID | (SEQ ID | (SEQ ID | (SEQ ID | (SEQ ID | |
| | NO: 228) | NO: 229) | NO: 230) | NO: 231) | NO: 232) | |
| 28021 | QNAHRKT | TSGNLTR | LKQMLAV | RSDNLSR | DNSNRKT | N/A |
| | (SEQ ID | (SEQ ID | (SEQ ID | (SEQ ID | (SEQ ID | |
| | NO: 233) | NO: 234) | NO: 235) | NO: 236) | NO: 237) | |
| 28022 | RSDNLSV | QNANRIT | TSGSLSR | QSSVRNS | DRSALAR | N/A |
| | (SEQ ID | (SEQ ID | (SEQ ID | (SEQ ID | (SEQ ID | |
| | NO: 238) | NO: 239) | NO: 240) | NO: 241) | NO: 242) | |
| 28023 | RSDNLSR | DNSNRKT | DRSNLTR | RSDVLSE | TRNGLKY | N/A |
| | (SEQ ID | (SEQ ID | (SEQ ID | (SEQ ID | (SEQ ID | |
| | NO: 243) | NO: 244) | NO: 245) | NO: 246) | NO: 247) | |
| 28024 | RSDALAR | RSDVLSE | RSSDRTK | RSDNLSV | QNANRIT | N/A |
| | (SEQ ID | (SEQ ID | (SEQ ID | (SEQ ID | (SEQ ID | |
| | NO: 248) | NO: 249) | NO: 250) | NO: 251) | NO: 252) | |
| 28025 | QSSDLSR | QSTHRNA | RSDNLAR | QRGNRNT | RSDHLSR | RNQDRTN |
| | (SEQ ID | (SEQ ID | (SEQ ID | (SEQ ID | (SEQ ID | (SEQ ID |
| | NO: 253) | NO: 254) | NO: 255) | NO: 256) | NO: 257) | NO: 258) |
| 28026 | DRSNLSR | RQDSRSQ | QSSDLSR | DRSALAR | TSGSLTR | N/A |
| | (SEQ ID | (SEQ ID | (SEQ ID | (SEQ ID | (SEQ ID | |
| | NO: 259) | NO: 260) | NO: 261) | NO: 262) | NO: 263) | |
| 28035 | QSSDLSR | AASNRSK | TSGSLSR | RSDALAR | RSDTLSQ | QRDHRIK |
| | (SEQ ID | (SEQ ID | (SEQ ID | (SEQ ID | (SEQ ID | (SEQ ID |
| | NO: 264) | NO: 265) | NO: 266) | NO: 267) | NO: 268) | NO: 269) |
| 28036 | RSDDLTR | QSSDLRR | RSDHLSA | QHGALQT | TSGNLTR | QSGHLSR |
| | (SEQ ID | (SEQ ID | (SEQ ID | (SEQ ID | (SEQ ID | (SEQ ID |
| | NO: 270) | NO: 271) | NO: 272) | NO: 273) | NO: 274) | NO: 275) |
| 28039 | TSGSLSR | RSDALAR | RSDTLSQ | QRDHRIK | TSGNLTR | DRGDLRK |
| | (SEQ ID | (SEQ ID | (SEQ ID | (SEQ ID | (SEQ ID | (SEQ ID |
| | NO: 276) | NO: 277) | NO: 278) | NO: 279) | NO: 280) | NO: 281) |
| 28040 | DSSDRKK | TSGNLTR | DNYNRAK | DRSHLTR | RSDNLTT | N/A |
| | (SEQ ID | (SEQ ID | (SEQ ID | (SEQ ID | (SEQ ID | |
| | NO: 282) | NO: 283) | NO: 284) | NO: 285) | NO: 286) | |
| 28051 | RSDNLSN | TSSSRIN | RSDNLSE | ASKTRKN | RSDALTQ | N/A |
| | (SEQ ID | (SEQ ID | (SEQ ID | (SEQ ID | (SEQ ID | |
| | NO: 287) | NO: 288) | NO: 289) | NO: 290) | NO: 291) | |
| 28052 | RSDTLST | DRSSRIK | RSDDLSK | DNSNRIK | N/A | N/A |
| | (SEQ ID | (SEQ ID | (SEQ ID | (SEQ ID | | |
| | NO: 292) | NO: 293) | NO: 294) | NO: 295) | | |
| 28053 | QSSDLSR | QAGNLSK | QSGDLTR | TSGSLSR | QSGNLAR | N/A |
| | (SEQ ID | (SEQ ID | (SEQ ID | (SEQ ID | (SEQ ID | |
| | NO: 296) | NO: 297) | NO: 298) | NO: 299) | NO: 300) | |
| 28054 | TSGSLSR | LRQTLRD | TSGNLTR | DRSALAR | RSDVLSE | RNFSLTM |
| | (SEQ ID | (SEQ ID | (SEQ ID | (SEQ ID | (SEQ ID | (SEQ ID |
| | NO: 301) | NO: 302) | NO: 303) | NO: 304) | NO: 305) | NO: 306) |
| 28055 | QSGDLTR | TSGSLSR | QSGNLAR | TSGSLSR | QSGSLTR | N/A |
| | (SEQ ID | (SEQ ID | (SEQ ID | (SEQ ID | (SEQ ID | |
| | NO: 307) | NO: 308) | NO: 309) | NO: 310) | NO: 311) | |
| 28056 | DRSALAR | TSGSLSR | LRQTLRD | TSGNLTR | DRSALAR | N/A |
| | (SEQ ID | (SEQ ID | (SEQ ID | (SEQ ID | (SEQ ID | |
| | NO: 312) | NO: 313) | NO: 314) | NO: 315) | NO: 316) | |

TABLE 7
Target Sites of FAD3 Zinc Fingers

| ZFP | Target Site (5′ to 3′) | SEQ ID NO: |
|---|---|---|
| 27961 | cgCCGGAGAAAGAGAGAGAGctttgagg | SEQ ID NO: 36 |
| 27962 | tgGTTGTCGCTATGGACcagcgtagcaa | SEQ ID NO: 37 |
| 27969 | tcTCCGTTcGCATTGcTACGCTggtcca | SEQ ID NO: 38 |
| 27970 | gaAAGGTTtGATCCGAGCGCAcaaccac | SEQ ID NO: 39 |
| 27973 | ctTGAACGGTGGTTgTGCGCTcggatca | SEQ ID NO: 40 |
| 27974 | tcGGAGATATAAGGGCGGCCattcctaa | SEQ ID NO: 41 |
| 27987 | taGCCCAGAACAGGGTTecttgggeggc | SEQ ID NO: 42 |
| 27988 | ctTCGTACTCGGCCACGactggtaattt | SEQ ID NO: 43 |
| 27989 | ttGAAGTTGCAaTAAGCTttctctcgct | SEQ ID NO: 44 |
| 27990 | acTTGCTGGTCGATCATGTTggccactc | SEQ ID NO: 45 |
| 27991 | aaGTAGTTGAAGTTGCAataagctttct | SEQ ID NO: 46 |
| 27992 | tgGTCGATCATGTTGGCcactcttgttt | SEQ ID NO: 47 |
| 28004 | aaCGAGAATGAAGGAATGAAgaatatga | SEQ ID NO: 48 |
| 28005 | atACCATGGTTGGTAAGtcattta | SEQ ID NO: 49 |
| 28021 | ccAACGAGgAATGATAGAtaaacaagag | SEQ ID NO: 50 |
| 28022 | caGTCACAGTTcTAAAAGtctatggtgt | SEQ ID NO: 51 |
| 28023 | tgTGACTGGACcAACGAGgaatgataga | SEQ ID NO: 52 |
| 28024 | tcTAAAAGTCTATGGTGttccttacatt | SEQ ID NO: 53 |
| 28025 | cgCCGGAGAAAGAGAGAGCTttgaggga | SEQ ID NO: 54 |
| 28026 | tgGTTGTCGCTATGGACcagcgtagcaa | SEQ ID NO: 55 |
| 28035 | ctTAAACGGTGGTTgTGCGCTcggatca | SEQ ID NO: 56 |
| 28036 | tcGGAGATATAAGGGCTGCGattcctaa | SEQ ID NO: 57 |
| 28039 | tcTCCGATctTAAACGGTGGTTgtgcgc | SEQ ID NO: 58 |
| 28040 | atAAGGGCTGCGATTCCtaagcattgtt | SEQ ID NO: 59 |
| 28051 | agATGGCCCAGAAAAGGgttccttgggc | SEQ ID NO: 60 |
| 28052 | cgTACTCGGCCACGactggtaatttaat | SEQ ID NO: 61 |
| 28053 | ttGAAGTTGCAaTAAGCTttctctcgct | SEQ ID NO: 62 |
| 28054 | acTTGCTGGTCGATCGTGTTggccactc | SEQ ID NO: 63 |
| 28055 | aaGTAGTTGAAGTTGCAataagctttct | SEQ ID NO: 64 |
| 28056 | tgGTCGATCGTGTTGGCcactcttgttt | SEQ ID NO: 65 |

TABLE 8
FAD2 Zinc Finger Designs

| ZFP | F1 | F2 | F3 | F4 | F5 | F6 |
|---|---|---|---|---|---|---|
| 24800 | RSDNLST | HSHARIK | HRSSLRR | RSDHLSE | QNANRIT | N/A |
| | (SEQ ID | (SEQ ID | (SEQ ID | (SEQ ID | (SEQ ID | |
| | NO: 317) | NO: 318) | NO: 319) | NO: 320) | NO: 321) | |
| 24801D | RSNLSR | HRSSLRR | TSGNLTR | MSHHLRD | DQSNLRA | N/A |
| | (SEQ ID | (SEQ ID | (SEQ ID | (SEQ ID | (SEQ ID | |
| | NO: 322) | NO: 323) | NO: 324) | NO: 325) | NO: 326) | |
| 24794 | QSGNLAR | RSDNLSR | DNNARIN | DRSNLSR | RSDHLTQ | N/A |
| | (SEQ ID | (SEQ ID | (SEQ ID | (SEQ ID | (SEQ ID | |
| | NO: 327) | NO: 328) | NO: 329) | NO: 330) | NO: 331) | |
| 24795 | RSDNLRE | QSGALAR | QSGNLAR | RSDVLSE | SPSSRRT | N/A |
| | (SEQ ID | (SEQ ID | (SEQ ID | (SEQ ID | (SEQ ID | |
| | NO: 332) | NO: 333) | NO: 334) | NO: 335) | NO: 336) | |
| 24810 | RSDSLSR | RKDARIT | RSDHLSA | WSSSLYY | NSRNLRN | N/A |
| | (SEQ ID | (SEQ ID | (SEQ ID | (SEQ ID | (SEQ ID | |
| | NO: 337) | NO: 338)** | NO: 339)** | NO: 340)** | NO: 341)** | |
| 24811 | DQSTLRN | DRSNLSR | DRSNLWR | DRSALSR | RSDALAR | N/A |
| | (SEQ ID | (SEQ ID | (SEQ ID | (SEQ ID | (SEQ ID | |
| | NO: 342) | NO: 343) | NO: 344) | NO: 345) | NO: 346) | |
| 24814 | RSDALSR | DRSDLSR | RSDHLTQ | QSGALAR | QSGNLAR | N/A |
| | (SEQ ID | (SEQ ID | (SEQ ID | (SEQ ID | (SEQ ID | |
| | NO: 347) | NO: 348) | NO: 349) | NO: 350) | NO: 351) | |
| 24815 | DRSNLSR | DSSARNT | DRSSRKR | QSGDLTR | LAHHLVQ | N/A |
| | (SEQ ID | (SEQ ID | (SEQ ID | (SEQ ID | (SEQ ID | |
| | NO: 352) | NO: 353) | NO: 354) | NO: 355) | NO: 356) | |
| 24818 | RSDNLST | HSHARIK | TSGHLSR | RSDNLSV | IRSTLRD | N/A |
| | (SEQ ID | (SEQ ID | (SEQ ID | (SEQ ID | (SEQ ID | |
| | NO: 357) | NO: 358) | NO: 359) | NO: 360) | NO: 361) | |
| 24819 | TSGHLSR | DRSNLSR | HRSSLRR | TSGNLTR | MSHHLRD | N/A |
| | (SEQ ID | (SEQ ID | (SEQ ID | (SEQ ID | (SEQ ID | |
| | NO: 362) | NO: 363) | NO: 364) | NO: 365) | NO: 366) | |
| 24796 | RSDALSR | DRSDLSR | RSDHLTQ | QSGALAR | QSGNLAR | N/A |
| | (SEQ ID | (SEQ ID | (SEQ ID | (SEQ ID | (SEQ ID | |
| | NO: 367) | NO: 368) | NO: 369) | NO: 370) | NO: 371) | |
| 24797 | RSAVLSE | TNSNRIT | LKQHLNE | QSGALAR | QSGNLAR | N/A |
| | (SEQ ID | (SEQ ID | (SEQ ID | (SEQ ID | (SEQ ID | |
| | NO: 372) | NO: 373) | NO: 374) | NO: 375) | NO: 376) | |
| 24836 | DRSNLSR | QSGDLTR | QSGALAR | DRSNLSR | QRTHLTQ | N/A |
| | (SEQ ID | (SEQ ID | (SEQ ID | (SEQ ID | (SEQ ID | |
| | NO: 377) | NO: 378) | NO: 379) | NO: 380) | NO: 381) | |
| 24837 | RSDNLSN | TNSNRIK | QSSDLSR | QSSDLRR | DRSNRIK | N/A |
| | (SEQ ID | (SEQ ID | (SEQ ID | (SEQ ID | (SEQ ID | |
| | NO: 382) | NO: 383) | NO: 384) | NO: 385) | NO: 386) | |
| 24844 | RSANLAR | RSDNLTT | QSGELIN | RSADLSR | RSDNLSE | DRSHLAR |
| | (SEQ ID | (SEQ ID | (SEQ ID | (SEQ ID | (SEQ ID | (SEQ ID |
| | NO: 387) | NO: 388) | NO: 389) | NO: 390) | NO: 391) | NO: 392) |
| 24845 | DRSHLAR | RSDNLSE | SKQYLIK | ERGTLAR | RSDHLTT | N/A |
| | (SEQ ID | (SEQ ID | (SEQ ID | (SEQ ID | (SEQ ID | |
| | NO: 393) | NO: 394) | NO: 395) | NO: 396) | NO: 397) | |
| 24820 | QSGALAR | QSGNLAR | DRSHLAR | DRSDLSR | RSDNLTR | N/A |
| | (SEQ ID | (SEQ ID | (SEQ ID | (SEQ ID | (SEQ ID | |
| | NO: 398) | NO: 399) | NO: 400) | NO: 401) | NO: 402) | |
| 24821 | DRSHLAR | RSDNLSE | SKQYLIK | ERGTLAR | RSDHLTT | N/A |
| | (SEQ ID | (SEQ ID | (SEQ ID | (SEQ ID | (SEQ ID | |
| | NO: 403) | NO: 404) | NO: 405) | NO: 406) | NO: 407) | |
| 24828 | DRSDLSR | RSDNLTR | QRTHLTQ | RSDNLSE | ASKTRKN | N/A |
| | (SEQ ID | (SEQ ID | (SEQ ID | (SEQ ID | (SEQ ID | |
| | NO: 408) | NO: 409) | NO: 410) | NO: 411) | NO: 412) | |
| 24829 | RSDTLSE | QSHNRTK | QSDHLTQ | RSSDLSR | QSSDLSR | RSDHLTQ |
| | (SEQ ID | (SEQ ID | (SEQ ID | (SEQ ID | (SEQ ID | (SEQ ID |
| | NO: 413) | NO: 414) | NO: 415) | NO: 416) | NO: 417) | NO: 418) |
| 24832 | RSDSLSR | RKDARIT | DRSHLSR | QSGNLAR | QSSDLSR | DRSALAR |
| | (SEQ ID | (SEQ ID | (SEQ ID | (SEQ ID | (SEQ ID | (SEQ ID |
| | NO: 419) | NO: 420) | NO: 421) | NO: 422) | NO: 423) | NO: 424) |
| 24833 | RSDDLSK | RSDTRKT | DRSNLSR | DRSNLWR | RSDSLSR | NNDHRKT |
| | (SEQ ID | (SEQ ID | (SEQ ID | (SEQ ID | (SEQ ID | (SEQ ID |
| | NO: 425) | NO: 426 | NO: 427) | NO: 428) | NO: 429) | NO: 430) |

TABLE 9
Target Sites of FAD2 Zinc Fingers

| | | | ZFP target/ |
|---|---|---|---|
| | | | binding site |
| | | | present in |
| ZFP | Plasmid No. | Target Site (5′ to 3′) | SEQ ID Nos. |
| 24800 | pDAB104001 | ccCAAAGGGTTGTTGAGgtacttgccgt | SEQ ID NO: 66 |
| 24801 | pDAB104001 | cgCACCGTGATGTTAACggttcagttca | SEQ ID NO: 67 |
| 24794 | pDAB104002 | taAGGGACGAGGAGGAAggagtggaaga | SEQ ID NO: 68 |
| 24795 | pDAB104002 | ttCTCCTGGAAGTACAGtcatcgacgcc | SEQ ID NO: 69 |
| 24810 | pDAB104003 | gtCGCTGAAGGcGTGGTGgccgcactcg | SEQ ID NO: 70 |
| 24811 | pDAB104003 | caGTGGCTgGACGACACCgtcggcctca | SEQ ID NO: 71 |
| 24814 | pDAB104004 | gaGAAGTAAGGGACGAGgaggaaggagt | SEQ ID NO: 72 |
| 24815 | pDAB104004 | gaAGTACAGTCATCGACgccaccattcc | SEQ ID NO: 73 |
| 24818 | pDAB104005 | tcCCAAAGGGTtGTTGAGgtacttgccg | SEQ ID NO: 74 |
| 24819 | pDAB104005 | acCGTGATGTTAACGGTtcagttcactc | SEQ ID NO: 75 |
| 24796 | pDAB104006 | gaGAAGTAAGGGACGAGgaggaaggagt | SEQ ID NO: 76 |
| 24797 | pDAB104006 | tgGAAGTAcAGTCATCGAcgccaccatt | SEQ ID NO: 77 |
| 24836 | pDAB104007 | gtAGAGACcGTAGCAGACggcgaggatg | SEQ ID NO: 78 |
| 24837 | pDAB104007 | gcTACGCTGCTgTCCAAGgagttgcctc | SEQ ID NO: 79 |
| 24844 | pDAB104008 | gaGGCCAGGCGAAGTAGGAGagagggtg | SEQ ID NO: 80 |
| 24845 | pDAB104008 | acTGGGCCTGCCAGGGCtgegtectaac | SEQ ID NO: 81 |
| 24820 | pDAB104009 | gaGAGGCCaGGCGAAGTAggagagaggg | SEQ ID NO: 82 |
| 24821 | pDAB104009 | acTGGGCCTGCCAGGGCtgcgtcctaac | SEQ ID NO: 83 |
| 24828 | pDAB104010 | agGCCCAGtAGAGAGGCCaggcgaagta | SEQ ID NO: 84 |
| 24829 | pDAB104010 | ccAGGGCTGCGTCCTAACCGgcgtctgg | SEQ ID NO: 85 |
| 24832 | pDAB104011 | taGTCGCTGAAGGCGTGGTGgccgcact | SEQ ID NO: 86 |
| 24833 | pDAB104011 | agTGGCTGGACGACaCCGTCGgcctcat | SEQ ID NO: 87 |

### Example 3

### Evaluation of Zinc Finger Nuclease Cleavage of Fad2 Genes

Construct Assembly

Plasmid vectors containing ZFN expression constructs of the exemplary zinc finger nucleases, which were identified using the yeast assay, as described in Example 2, were designed and completed using skills and techniques commonly known in the art. Each zinc finger-encoding sequence was fused to a sequence encoding an opaque-2 nuclear localization signal (Maddaloni et al. (1989) Nuc. Acids Res. 17(18):7532), that was positioned upstream of the zinc finger nuclease.

Next, the opaque-2 nuclear localization signal::zinc finger nuclease fusion sequence was paired with the complementary opaque-2 nuclear localization signal::zinc finger nuclease fusion sequence. As such, each construct consisted of a single open reading frame comprised of two opaque-2 nuclear localization signal::zinc finger nuclease fusion sequences separated by the 2A sequence from Thosea asigna virus (Mattion et al. (1996) J. Virol. 70:8124-8127). Expression of the fusion proteins was driven by a relatively strong constitutive promoter such as a promoter derived from the Cassaya Vein Mosaic Virus (CsVMV) promoter and flanked by the Agrobacterium tumefaciens ORF23 3′ UnTranslated Region (AtuORF23 3′UTR).

The vectors were assembled using the IN-FUSION™ Advantage Technology (Clontech, Mountain View, Calif.). Restriction endonucleases were obtained from New England BioLabs (NEB; Ipswich, Mass.) and T4 DNA Ligase (Invitrogen) was used for DNA ligation. Plasmid preparations were performed using NUCLEOSPIN® Plasmid Kit (Macherey-Nagel Inc., Bethlehem, Pa.) or the Plasmid Midi Kit (Qiagen) following the instructions of the suppliers. DNA fragments were isolated using QIAquick Gel Extraction Kit™ (Qiagen) after agarose Tris-acetate gel electrophoresis. Colonies of all assembled plasmids were initially screened by restriction digestion of miniprep DNA. Plasmid DNA of selected clones was sequenced by a commercial sequencing vendor (Eurofins MWG Operon, Huntsville, Ala.). Sequence data were assembled and analyzed using the SEQUENCHER™ software (Gene Codes Corp., Ann Arbor, Mich.). Before delivery to B. napus protoplasts, Plasmid DNA was prepared from cultures of E. coli using the Pure Yield PLASMID MAXIPREP System® (Promega Corporation, Madison, Wis.) or PLASMID MAXI KIT® (Qiagen, Valencia, Calif.) following the instructions of the suppliers.

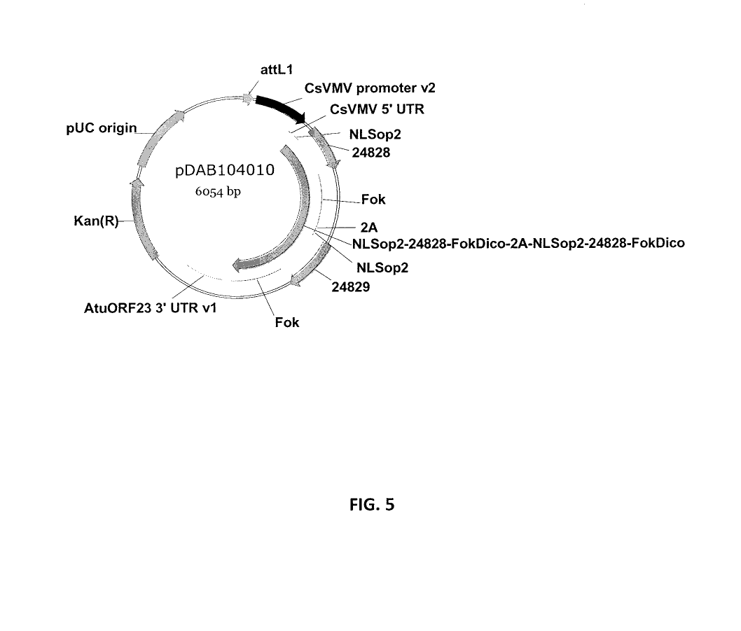

The resulting eleven plasmid constructs; pDAB104008 (containing the ZFN24845 and ZFN24844 construct), pDAB104009 (containing the ZFN24820 and ZFN24821 construct), pDAB104010 (containing the ZFN24828 and ZFN24829 construct) (FIG. 5), pDAB104003 (containing the ZFN24810 and ZFN24811 construct), pDAB104011 (containing the ZFN24832 and ZFN24833 construct), pDAB104002 (containing the ZFN24794 and ZFN24795 construct), pDAB 104006 (containing the ZFN24796 and ZFN24797 construct), pDAB 104004 (containing the ZFN24814 and ZFN24815 construct), pDAB 104001 (containing the ZFN24800 and ZFN24801 construct), pDAB104005 (containing the ZFN24818 and ZFN24819 construct), and pDAB104007 (containing the ZFN24836 and ZFN24837 construct) were confirmed via restriction enzyme digestion and via DNA sequencing. Table 10 lists the different constructs and the specific FAD sequence which each ZFN was designed to cleave and bind.

The resulting plasmid constructs; pDAB107824 (ZFNs 28025-2A-28026), pDAB107815 (ZFNs 27961-2A-27962), pDAB107816 (ZFNs 27969-2A-27970), pDAB107817 (ZFNs 27973-2A-27974), pDAB107825 (ZFNs 28035-2A-28036), pDAB107826 (ZFNs 28039-2A-28040), pDAB107818 (ZFNs 27987-2A-27988), pDAB107827 (ZFNs 28051-2A-28052), pDAB 107821 (ZFNs 28004-2A-28005), pDAB 107819 (ZFNs 27989-2A-27990), pDAB 107828 (ZFNs 28053-2A-28054), pDAB107829 (ZFNs 28055-2A-28056), pDAB107820 (ZFNs 27991-2A-27992), pDAB107822 (ZFNs 28021-2A-28022) and pDAB107823 (ZFNs 28023-2A-28024) were confirmed via restriction enzyme digestion and via DNA sequencing.

TABLE 10
lists the Zinc Finger protein binding motif and the corresponding
construct number. Each Zinc Finger was designed to bind and
cleave the FAD2A which is described in the table.

| | | | Target Cut Site in |
|---|---|---|---|
| ZFN Design | Construct No. | Locus ID. | FAD2A Sequence |
| 24844-2A-24845 | pDAB104008 | FAD2_ZFN_Locus1_F2A | 263-265 |
| 24820-2A-24821 | pDAB104009 | FAD2_ZFN_Locus1_F2B | 265 |
| 24828-2A-24829 | pDAB104010 | FAD2_ZFN_Locus1_F2C | 275 |
| 24810-2A-24811 | pDAB104003 | FAD2_ZFN_Locus2_F1D | 343-345 |
| 24832-2A-24833 | pDAB104011 | FAD2_ZFN_Locus2_F1E | 345-346 |
| 24794-2A-24795 | pDAB104002 | FAD2_ZFN_Locus3_F2F | 402 |
| 24796-2A-24797 | pDAB104006 | FAD2_ZFN_Locus3_F2G | 408 |
| 24814-2A-24815 | pDAB104004 | FAD2_ZFN_Locus3_F2H | 408-410 |
| 24800-2A-24801 | pDAB104001 | FAD2_ZFN_Locus4_F1J | 531 |
| 24818-2A-24819 | pDAB104005 | FAD2_ZFN_Locus4_F1K | 532-534 |
| 24836-2A-24837 | pDAB104007 | FAD2_ZFN_Locus5_F1L | 724 |

Preparation of DNA for Transfection

Plasmid DNA of the above described vectors was sterilized by precipitation and washing in 100% (v/v) ethanol and dried in a laminar flow hood. The DNA pellet was suspended in 30 μL of sterile double-distilled water at a final concentration of 0.7 μg/μl for transfection into protoplast cells as described below. The preparation of the plasmid DNA was undertaken to result in supercoiled plasmid DNA for transient transfection and linearized plasmid DNA for stable transfection. The addition of carrier DNA (e.g. fish-sperm DNA) to the transforming plasmid was not required for the transient transfection of protoplast cells. For transient studies about 30 μg of plasmid DNA per 10⁶ protoplasts was used per transformation.

Transfection

Transfection of Brassica napus L. var. DH10275 was completed as described in Spangenberg et al., (1986) Plant Physiology 66: 1-8, the media formulations are described in Spangenberg G. and Protrykus I. (1995) Polyethylene Glycol-Mediated Direct Gene Transfer in Tobacco Protoplasts. In: Gene Transfer to Plants. (Protrykus I. and Spangenberg G. Eds.) Springer-Verlag, Berlin. Brassica napus seeds were surface sterilized in 70% ethanol. The seeds were immersed in 12 mL of the 70% ethanol solution and mixed by gently rocking the cocktail for 10 minutes. The 70% ethanol solution was removed by decanting the solution and exchanged with a seed sterilization solution consisting of 1% w/v calcium hypochlorite and 0.1% v/v Tween-20. The seeds were immersed in the seed sterilization solution and mixed by gently rocking the cocktail for 25 minutes. The seed sterilization solution was decanted and the sterilized seeds were rinsed three times in 50 mL of sterile water. Finally, the seeds were transferred to a sterile 80 mm WHATMAN® filter paper disc (Fisher-Scientific, St. Louis, Mo.) that had been laid within a Petri dish and the seeds were lightly saturated with sterile water. The Petri dish was sealed with PARAFILM® (Fisher-Scientific, St. Louis, Mo.) and the plates were incubated at 25° C. under complete darkness for one to two days. After signs of seedling emergence were observed from the seeds, the seedlings were transferred to Petri dish containing solidified GEM medium to encourage further seed germination. The seedlings were incubated on the GEM medium at 25° C. for four to five days.

A volume of liquid PS medium (about 10 mL) was decanted into a sterile Petri dish. Using sterile forceps and a scalpel, an aerial portion of the four to five day old seedling in the 4-leaf stage of growth and development, was removed and discarded. Hypocotyl segments in lengths of 20-40 mm were determined to produce the highest population of small, cytoplasmic-rich protoplasts. The hypocotyl segments were aseptically excised and transferred to liquid PS medium. The excised hypocotyl segments were grouped together and cut transversely into 5-10 mm segments. Next, the hypocotyl segments were transferred to fresh PS medium and incubated at room temperature for 1 hour. The plasmolyzed hypocotyls were transferred to a Petri dish containing enzyme solution. Care was taken to immerse all of the hypocotyl segments into the solution. The Petri dishes were sealed with PARAFILM® and incubated overnight for sixteen to eighteen hours at 20-22° C. with gentle rocking.

Protoplast cells were released from the hypocotyl segments. The overnight hypocotyl digests were gently agitated to release protoplasts into the enzyme solution. The Petri dish was angled slightly to aid the transfer of the digesting suspension which consisted of enzyme solution and plant debris. Using a 10 mL pipette the digesting suspension was transferred to a sterilized protoplast filtration (a filter of 100 micron mesh) unit to further separate the protoplasts from the plant debris. The filtration unit was tapped gently to release the excess liquid that had been caught in the sieve. The protoplast suspension, about 8 to 9 mL, was gently mixed and distributed into 14 mL sterile plastic round-bottomed centrifuge tubes. Each suspension was overlaid with 1.5 mL of W5 solution. The W5 solution was carefully dispensed over the protoplast suspension at an angle and dispensed drop-by-drop with minimal agitation. The addition of the W5 solution to the protoplast suspension resulted in the production of a protoplast rich interface. This interface was collected using a pipette. Next, the collected protoplasts were transferred into a new 14 mL centrifuge tube, and gently mixed. The yield or obtained protoplasts were determined using a hemocytometer to determine the number of protoplasts per milliliter. The method was repeated, wherein leaf tissue was digested to produce mesophyll protoplasts.

Next, W5 solution was added to a volume of 10 mL and the protoplasts were pelleted at 70 g, before removing the W5 solution. The remaining protoplast suspension was resuspended by gentle shaking. Each tube containing the protoplast suspension was filled with 5 mL of W5 solution and incubated at room temperature from one to four hours. The protoplast suspensions were pelleted at 70 g, and all of the W5 solution was removed. Next, 300 μL of transformation buffer was added to each of the pelleted protoplast suspensions which contained the isolated protoplasts. To each of the tubes, 10 μg of plasmid DNA was added to the protoplast suspensions. The plasmid DNA consisted of the Zinc Finger Nuclease constructs described above (e.g., pDAB104010). Next, 300 μL of pre-warmed PEG 4000 solution was added to the protoplast suspension and the tubes were gently tapped. The protoplast suspensions and transformation mixture was allowed to incubate at room temperature for fifteen minutes without any agitation. An additional 10 mL of W5 solution was added to each tube in sequential aliquots of 1 mL, 1 mL, 1 mL, 2 mL, 2 mL, and 3 mL with gentle inversion of the tubes between each addition of W5 solution. The protoplasts were pelleted by spinning in a centrifuge at 70 g. All of the W5 solution was removed leaving a pure protoplast suspension.

Next, 0.5 mL of K3 medium was added to the pelleted protoplast cells and the cells were resuspended. The resuspended protoplast cells were placed in the center of a Petri dish and 5 mL of K3 and 0.6 mL Sea Plaque™ agarose (Cambrex, East Rutherford, N.J.) in a 1:1 concentration. The Petri dishes were shaken in a single gentle swirling motion and left to incubate for 20-30 minutes at room temperature. The Petri dishes were sealed with PARAFILM® and the protoplasts were cultured for twenty-four hours in complete darkness. After the incubation in darkness, the Petri dishes were cultured for six days in dim light (5 μMol m⁻² s⁻¹ of Osram L36 W/21 Lumilux white tubes). After the culture step, a sterile spatula was used to divide the agarose containing the protoplasts into quadrants. The separated quadrants were placed into a 250 mL plastic culture vessel containing 20 mL of A medium and incubated on a rotary shaker at 80 rpm and 1.25 cm throw at 24° C. in continuous dim light for 14 days and then analyzed to determine the level of activity of each Zinc Finger Nuclease construct.

Genomic DNA Isolation from Canola Protoplasts

Transfected protoplasts were supplied in individual 1.5 or 2.0 mL microfuge tubes. The cells were pelleted at the base of the tube in a buffer solution. DNA extraction was carried out by snap freezing the cells in liquid nitrogen followed by freeze drying the cells, for about 48 hours in A LABCONCO FREEZONE 4.5® (Labconco, Kansas City, Mo.) at −40° C. and about 133×10⁻³ mBar pressure. The lyophilized cells were subjected to DNA extraction using the DNEASY® (QIAGEN, Carlsbad, Calif.) plant kit following manufactures instructions, with the exception that tissue disruption was not required and the protoplast cells were added directly to the lysis buffer.

Testing of Fad2a and Fad3 ZFNs for Genomic DNA Sequence Cleavage in Canola Protoplasts

The design of the ZFN target sites for the FAD2A and FAD3 gene loci were clustered, so that multiple pairs of ZFN were design to overlap the target sites. The clustering of ZFN target sites enabled PCR primers to be designed that would amplify the surrounding genomic sequence from all FAD2A and FAD3 gene family members within a 100 bp window as to encapsulate all of the overlapping ZFN target sites. As such, the Illumina short read sequence technology could be used to assess the integrity of the target ZFN site of the transfected protoplasts. In addition, the PCR primers designed needed to include specific nucleotide bases that would attribute sequence reads to the specific gene member of the FAD2A and FAD3 family. Therefore, all of the PCR primers would be required to bind 5-10 nucleotides away from any ZFN target cut site as non-homologous end joining (NHEJ) activity is known to cause small deletions that could remove a priming site, inhibit amplification and therefore distort the assessment of NHEJ activity.

Primers were designed to bind to all of the ZFN target loci for the FAD2A and FAD3 gene families (Table 11) and were empirically tested for amplification of all gene family members through Sanger based sequencing of PCR amplification products. In several instances primers could not be developed that would distinguish all gene family members (Table 12 and Table 13), however in all instances the target gene sequences of FAD2A and FAD3, could be distinguished. Following PCR primer design custom DNA barcode sequences were incorporated into the PCR primers that were used to distinguish the different ZFN target loci and identify specific sequence reads to a transfection and ZFN (Tables 11, 12 and 13).

TABLE 11
Primer sequences designed for FAD2 and FAD3 ZFN assessment of activity.
Primers include custom barcodes, along with both requisite Illumina
adaptor sequences for construction of Illumina library for sequencing-
by-synthesis analysis. Purchased primer was the sum of all three
columns presented.

| | SEQ | | | |
|---|---|---|---|---|
| | ID | Illumina Adaptor | | |
| Locus ID | NO: | Primer Sequence | Barcode | Locus Primer |
| FAD2_ZFN_Locus | 88 | ACACTCTTTCCCTACACGACGCT | CGGG | CCCTCTCYCYTACYTC |
| 1_F | | CTTCCGATCT | | GCC |
| FAD2_ZFN_Locus | 89 | ACACTCTTTCCCTACACGACGCT | ACGTA | CCCTCTCYCYTACYTC |
| 1_F2A | | CTTCCGATCT | | GCC |
| FAD2_ZFN_Locus | 90 | ACACTCTTTCCCTACACGACGCT | CGTAC | CCCTCTCYCYTACYTC |
| 1_F2B | | CTTCCGATCT | | GCC |
| FAD2_ZFN_Locus | 91 | ACACTCTTTCCCTACACGACGCT | GTACG | CCCTCTCYCYTACYTC |
| 1_F2C | | CTTCCGATCT | | GCC |
| FAD2_ZFN_Locus | 92 | ACACTCTTTCCCTACACGACGCT | TACGT | GTCATAGCCCACGAGT |
| 2_F1D | | CTTCCGATCT | | GCGGC |
| FAD2_ZFN_Locus | 93 | ACACTCTTTCCCTACACGACGCT | CTGAC | GTCATAGCCCACGAGT |
| 2_F1E | | CTTCCGATCT | | GCGGC |
| FAD2_ZFN_Locus | 94 | ACACTCTTTCCCTACACGACGCT | TGACT | GTCGGCCTCATCTTCC |
| 3_F2F | | CTTCCGATCT | | ACTCC |
| FAD2_ZFN_Locus | 95 | ACACTCTTTCCCTACACGACGCT | GACTG | GTCGGCCTCATCTTCC |
| 3_F2G | | CTTCCGATCT | | ACTCC |
| FAD2_ZFN_Locus | 96 | ACACTCTTTCCCTACACGACGCT | ACTGA | GTCGGCCTCATCTTCC |
| 3_F2H | | CTTCCGATCT | | ACTCC |
| FAD2_ZFN_Locus | 97 | ACACTCTTTCCCTACACGACGCT | GCTAG | CAGACATCAAGTGGTA |
| 4_F1J | | CTTCCGATCT | | CGGC |
| FAD2_ZFN_Locus | 98 | ACACTCTTTCCCTACACGACGCT | CTAGC | CAGACATCAAGTGGTA |
| 4_F1K | | CTTCCGATCT | | CGGC |
| FAD2_ZFN_Locus | 99 | ACACTCTTTCCCTACACGACGCT | TAGCT | ATCTCCGACGCTGGCA |
| 5_F1L | | CTTCCGATCT | | TCCTC |
| FAD2_ZFN_Locus | 100 | CGGTCTCGGCATTCCTGCTGAAC | ACGTA | CTGGTAGTCGCTGAAG |
| 1_R1A | | CGCTCTTCCGATCT | | GCGT |
| FAD2_ZFN_Locus | 101 | CGGTCTCGGCATTCCTGCTGAAC | CGTAC | CTGGTAGTCGCTGAAG |
| 1_R1B | | CGCTCTTCCGATCT | | GCGT |
| FAD2_ZFN_Locus | 102 | CGGTCTCGGCATTCCTGCTGAAC | GTACG | CTGGTAGTCGCTGAAG |
| 1_R1C | | CGCTCTTCCGATCT | | GCGT |
| FAD2_ZFN_Locus | 103 | CGGTCTCGGCATTCCTGCTGAAC | TACGT | GGACGAGGAGGAAGG |
| 2_R1D | | CGCTCTTCCGATCT | | AGTGGA |
| FAD2_ZFN_Locus | 104 | CGGTCTCGGCATTCCTGCTGAAC | CTGAC | GGACGAGGAGGAAGG |
| 2_R1E | | CGCTCTTCCGATCT | | AGTGGA |
| FAD2_ZFN_Locus | 105 | CGGTCTCGGCATTCCTGCTGAAC | TGACT | AGTGTTGGAATGGTGG |
| 3_R1F | | CGCTCTTCCGATCT | | CGTCG |
| FAD2_ZFN_Locus | 106 | CGGTCTCGGCATTCCTGCTGAAC | GACTG | AGTGTTGGAATGGTGG |
| 3_R1G | | CGCTCTTCCGATCT | | CGTCG |
| FAD2_ZFN_Locus | 107 | CGGTCTCGGCATTCCTGCTGAAC | ACTGA | AGTGTTGGAATGGTGG |
| 3_R1H | | CGCTCTTCCGATCT | | CGTCG |
| FAD2_ZFN_Locus | 108 | CGGTCTCGGCATTCCTGCTGAAC | GCTAG | CCCGAGACGTTGAAG |
| 4_R1J | | CGCTCTTCCGATCT | | GCTAAG |
| FAD2_ZFN_Locus | 109 | CGGTCTCGGCATTCCTGCTGAAC | CTAGC | CCCGAGACGTTGAAG |
| 4_R1K | | CGCTCTTCCGATCT | | GCTAAG |
| FAD2_ZFN_Locus | 110 | CGGTCTCGGCATTCCTGCTGAAC | TAGCT | GAAGGATGCGTGTGCT |
| 5_R1L | | CGCTCTTCCGATCT | | GCAAG |
| FAD3_ZFN-Locus | 111 | ACACTCTTTCCCTACACGACGCT | ACGTA | CCTTTCTTCACCACATT |
| 1A_F3 | | CTTCCGATCT | | YCA |
| FAD3_ZFN_Locus | 112 | ACACTCTTTCCCTACACGACGCT | CGTAC | CCTTTCTTCACCACATT |
| 1B_F3 | | CTTCCGATCT | | YCA |
| FAD3_ZFN_Locus | 113 | ACACTCTTTCCCTACACGACGCT | CTGAC | GATGGTTGTCGCTATG |
| 2C_F1 | | CTTCCGATCT | | GACC |
| FAD3_ZFN_Locus | 114 | ACACTCTTTCCCTACACGACGCT | TGACT | CGAAAGGTTTGATCCR |
| 3D_F1 | | CTTCCGATCT | | AGCG |
| FAD3_ZFN_Locus | 115 | ACACTCTTTCCCTACACGACGCT | GACTG | CGAAAGGTTTGATCCR |
| 3E_F1 | | CTTCCGATCT | | AGCG |
| FAD3_ZFN_Locus | 116 | ACACTCTTTCCCTACACGACGCT | ACTGA | CGAAAGGTTTGATCCR |
| 3F_F1 | | CTTCCGATCT | | AGCG |
| FAD3_ZFN_Locus | 117 | ACACTCTTTCCCTACACGACGCT | GCTAG | CCGTGTATTTTGATAG |
| 4G_F1 | | CTTCCGATCT | | CTGGTTC |
| FAD3_ZFN_Locus | 118 | ACACTCTTTCCCTACACGACGCT | CTAGC | CCGTGTATTTTGATAG |
| 4H_F1 | | CTTCCGATCT | | CTGGTTC |
| FAD3_ZFN_Locus | 119 | ACACTCTTTCCCTACACGACGCT | TAGCT | GGAGCTTCTCAGACAT |
| 5J_F1 | | CTTCCGATCT | | TCCTCT |
| FAD3_ZFN_Locus | 120 | ACACTCTTTCCCTACACGACGCT | TCAGT | GTTTATTTGCCCCAAG |
| 6K_F1 | | CTTCCGATCT | | CGAGAG |
| FAD3_ZFN_Locus | 121 | ACACTCTTTCCCTACACGACGCT | CAGTC | GTTTATTTGCCCCAAG |
| 6L_F1 | | CTTCCGATCT | | CGAGAG |
| FAD3_ZFN_Locus | 122 | ACACTCTTTCCCTACACGACGCT | AGTCA | GTTTATTTGCCCCAAG |
| 6M_F1 | | CTTCCGATCT | | CGAGAG |
| FAD3_ZFN_Locus | 123 | ACACTCTTTCCCTACACGACGCT | GTCAG | GTTTATTTGCCCCAAG |
| 6N_F1 | | CTTCCGATCT | | CGAGAG |
| FAD3_ZFN_Locus | 124 | ACACTCTTTCCCTACACGACGCT | GTACG | ACTTCAACTACTTGCT |
| 7P_F3 | | CTTCCGATCT | | GGTCSAT |
| FAD3_ZFN_Locus | 125 | ACACTCTTTCCCTACACGACGCT | TACGT | ACTTCAACTACTTGCT |
| 7Q_F3 | | CTTCCGATCT | | GGTCSAT |
| FAD3_ZFN_Locus | 126 | CGGTCTCGGCATTCCTGCTGAAC | ACGTA | CGTTCACATTGSTRCG |
| 1A_R1 | | CGCTCTTCCGATCT | | YTGG |
| FAD3_ZFN_Locus | 127 | CGGTCTCGGCATTCCTGCTGAAC | CGTAC | CGTTCACATTGSTRCG |
| 1B_R1 | | CGCTCTTCCGATCT | | YTGG |
| FAD3_ZFN_Locus | 128 | CGGTCTCGGCATTCCTGCTGAAC | CTGAC | CCGATCTTAAACGGYG |
| 2C_R1 | | CGCTCTTCCGATCT | | GTTGT |
| FAD3_ZFN_Locus | 129 | CGGTCTCGGCATTCCTGCTGAAC | TGACT | TAGCTCATGGATCTCA |
| 3D_R1 | | CGCTCTTCCGATCT | | AAGGACT |
| FAD3_ZFN_Locus | 130 | CGGTCTCGGCATTCCTGCTGAAC | GACTG | TAGCTCATGGATCTCA |
| 3E_R1 | | CGCTCTTCCGATCT | | AAGGACT |
| FAD3_ZFN_Locus | 131 | CGGTCTCGGCATTCCTGCTGAAC | ACTGA | TAGCTCATGGATCTCA |
| 3F_R1 | | CGCTCTTCCGATCT | | AAGGACT |
| FAD3_ZFN_Locus | 132 | CGGTCTCGGCATTCCTGCTGAAC | GCTAG | TTAAATTACCAGTCGT |
| 4G_R_uni | | CGCTCTTCCGATCT | | GGCC |
| FAD3_ZFN_Locus | 133 | CGGTCTCGGCATTCCTGCTGAAC | CTAGC | TTAAATTACCAGTCGT |
| 4H_R_uni | | CGCTCTTCCGATCT | | GGCC |
| FAD3_ZFN_Locus | 134 | CGGTCTCGGCATTCCTGCTGAAC | TAGCT | CTTTTTTCTTCGATKCT |
| 5J_R2 | | CGCTCTTCCGATCT | | AAAGATT |
| FAD3_ZFN_Locus | 135 | CGGTCTCGGCATTCCTGCTGAAC | TCAGT | CTGTGACTGGACCAAC |
| 6K_R1 | | CGCTCTTCCGATCT | | GAGG |
| FAD3_ZFN_Locus | 136 | CGGTCTCGGCATTCCTGCTGAAC | CAGTC | CTGTGACTGGACCAAC |
| 6L_R1 | | CGCTCTTCCGATCT | | GAGG |
| FAD3_ZFN_Locus | 137 | CGGTCTCGGCATTCCTGCTGAAC | AGTCA | CTGTGACTGGACCAAC |
| 6M_R1 | | CGCTCTTCCGATCT | | GAGG |
| FAD3_ZFN_Locus | 138 | CGGTCTCGGCATTCCTGCTGAAC | GTCAG | CTGTGACTGGACCAAC |
| 6N_R1 | | CGCTCTTCCGATCT | | GAGG |
| FAD3_ZFN_Locus | 139 | CGGTCTCGGCATTCCTGCTGAAC | GTACG | ACTTACAATGTAAGGA |
| 7P_R1 | | CGCTCTTCCGATCT | | ACRCCRTA |
| FAD3_ZFN_Locus | 140 | CGGTCTCGGCATTCCTGCTGAAC | TACGT | ACTTACAATGTAAGGA |
| 7Q_1 | | CGCTCTTCCGATCT | | ACRCCRTA |

TABLE 12
Amplification performance of the designed PCR primers
on the FAD2 gene families. An “X” indicates gene copy detection
specificity, grey shading and an “+” indicates that at the specific
locus in question the sequence reads designed by the two
primers were unable to be distinguished and an “N/A” indicates
that the locus was unable to be amplified from those specific gene copies.

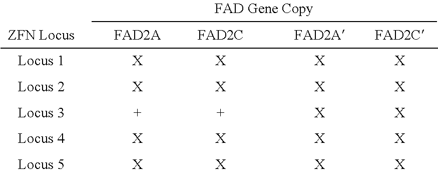

TABLE 13
Amplification performance of the designed PCR primers
on the FAD3 gene families. An “X” indicates gene copy detection
specificity, grey shading and an “+” indicates that at the specific
locus in question the sequence reads designed by the two
primers were unable to be distinguished and an “N/A” indicates that the
locus was unable to be amplified from those specific gene copies.

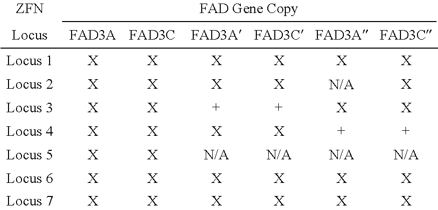

Following DNA extraction of canola protoplasts transfected with the ZFN, PCR amplification of the target ZFN loci was performed to generate the requisite loci specific DNA molecules in the correct format for Illumina based sequencing by synthesis technology. Each assay was optimised to work on 25 ng starting DNA (about 12,500 cell equivalents of the Brassica napus genome). Multiple reactions were performed, per sample to provide the coverage required to assess NHEJ efficiency and specificity at the appropriate level, about sixteen PCR reactions equivalent to 200,000 copies of the Brassica napus genome taken from individual protoplasts. PCR amplification master-mixes were made for all samples to be tested with the same assay and one reaction, performed in triplicate, was assayed using a quantitative PCR method that was used to determine the optimal number of cycles to perform on the target tissue, to ensure that PCR amplification had not become reagent limited and was still in an exponential amplification stage. The experimentation with the necessary negative control reactions, was performed in 96 well format using a MX3000P THERMOCYCLER® (Stratagene, LaJolla, Calif.). From the output gathered from the quantitative PCR platform, the relative increase in fluorescence was plotted from cycle-to-cycle and the cycle number was determined per assay that would deliver sufficient amplification, while not allowing the reaction to become reagent limited, in an attempt to reduce over cycling and the amplification of common transcripts or molecules. The unused master mix, remained on ice until the quantitative PCR analysis was concluded and the cycle number determined and was then aliquoted into the desired number of reaction tubes (about 16 per ZFN assay) and the PCR reaction was performed. Following amplification, samples for a single ZFN locus were pooled together and 200 μL of pooled product per ZFN was cleaned using the MINELUTE® PCR purification kit (Qiagen) following manufacturer's instructions. To enable the sample to be sequenced using the Illumina short read technology additional paired end primers were required to be attached by amplification onto the generated fragments. This was achieved by PCR amplification using primers that would be, in part complementary to the sequence added in the first round of amplification, but also contain the paired end sequence required. The optimal number of PCR cycles to perform, that would add the paired end sequences without over amplifying common fragments to the template was again determined using a sample pass through a quantitative PCR cycle analysis, as described previously. Following PCR amplification, the generated product was cleaned using a MINELUTE® column (Qiagen) following manufacturer's instructions and was resolved on a 2.5% agarose gel. DNA fragments visualised using SYBER SAFE® (Life Technologies, Carlsbad, Calif.) as bands of the correct size were gel extracted to remove any residual PCR generated primer-dimer or other spurious fragments, the DNA was extracted from the gel slice using a MINELUTE® gel extraction kit (Qiagen) following manufacturer's instructions. After completion of the gel extraction an additional clean up of the DNA was performed using AMPURE® magnetic beads (Beckman-Coulter, Brea, Calif.) with a DNA to bead ratio of 1:1.7. The DNA was then assessed for concentration using a quantitative PCR based library quantification kit for Illumina sequencing (KAPA) with a 1/40,000 and a 1/80,000 dilution and with the reaction being performed in triplicate. Based on the quantitative PCR results the DNA was diluted to a standard concentration of 2 nM and all libraries were combined for DNA sequencing. The samples were prepared for sequencing using a CBOT CLUSTER® generation kit (Illumina, San Diego, Calif.) and were sequenced on an ILLUMINA GA2x® with 100 bp paired-end sequencing reads following manufacturer's instructions.

Method of Data Analysis for Detection of Non-Homologous End Joining at Target Zinc Finger Sites

Following completion of the sequencing reaction and primary data calling performed using the Illumina bioinformatic pipeline for base calling, full analysis was performed to identify deleted bases at the target ZFN site in each instance. A custom PERL script was designed to extract and sort barcodes from DNA sequences computationally following a list of input sequences. The barcode had to match the reference sequence at a Phred score of greater than 30 to be accepted, to reduce misattributing sequence reads. After the sequence reads had been binned into the different barcode groups that had been used, a quality filter was passed across all sequences. The quality filter was a second custom developed PERL script. Sequence reads were excluded if there were more than three bases called as “N,” or if the median Phred score was less than 20, or if there were 3 consecutive bases with a Phred score of less than 20, or if the sequence read was shorter than 40 bp in length. The remaining sequences were merged where both of the paired sequence reads were available using the NEXTGENE® (SoftGenetics, State College, Pa.) package. The remaining merged sequence reads were then reduced to a collection of unique sequence reads using a third custom PERL script with a count of the number of redundant sequences that had been identified recorded on the end of the remaining sequence identifier. The unique sequence reads were then aligned to the FAD2 and FADS reference sequence using the NEXTGENE® software that created a gapped FASTA aligned file.

Using the gapped FASTA file a conversion of the gapped base position number to the input reference was performed using a fourth custom PERL script. This enabled bases that discriminate the different gene family members (either homoeologous or paralogous sequence variation between the different gene family members) to be identified in the assembled data. Once the conversion of base numbering had been performed it was possible to generate haplotype reports for each unique sequence reads and assign the reads to specific gene family members. Once the reads had been grouped by gene a 10 bp window was identified and assessed that surrounded the ZFN target site. The number of sequences with deletions was recorded per gene along with the number of missing bases.

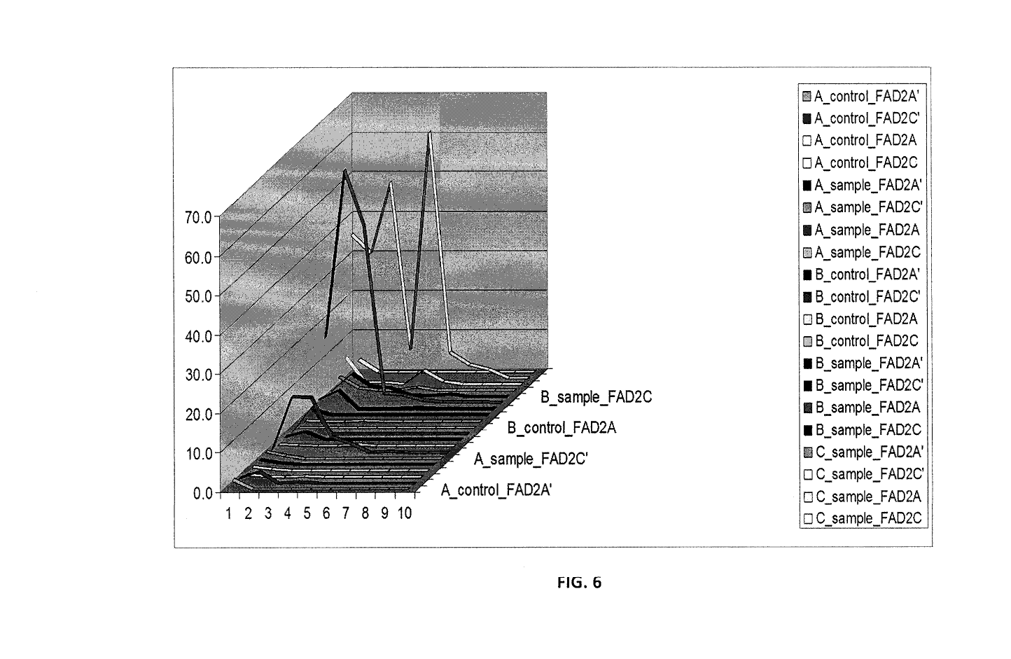

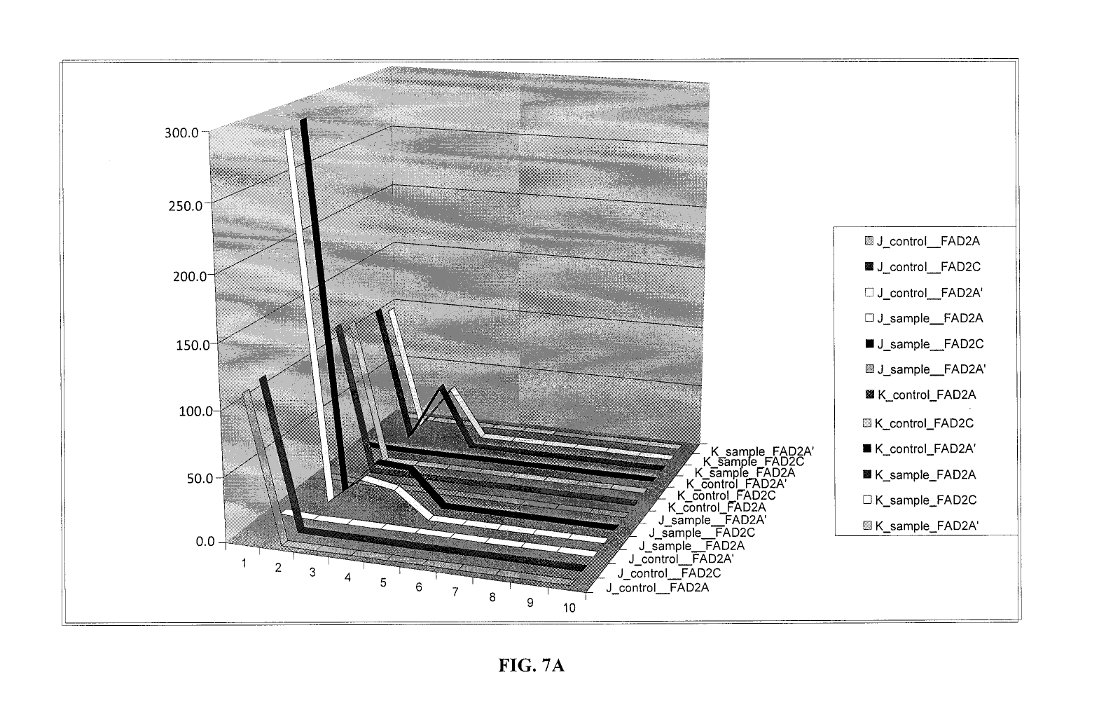

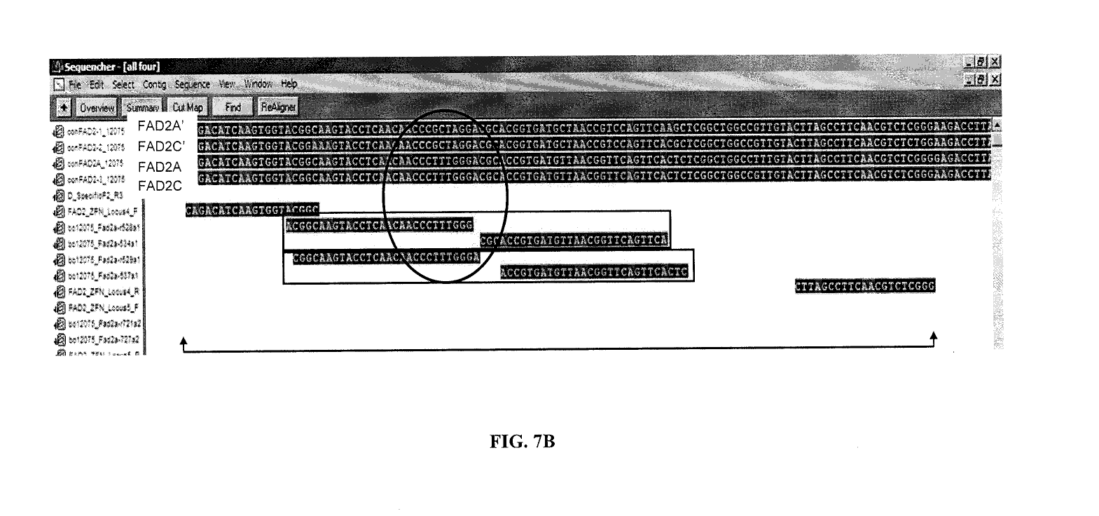

The data was then graphically displayed as a multiple line graph, with the number of sequences with 1 through 10 bases deleted at the target ZFN site per 10,000 sequence reads (FIG. 6). This analysis was performed for all ZFN transfections along with control transfections. In several instances, repeats in the native DNA sequence lead to an increase in sequencing error in the target ZFN site, such an error can be commonly seen as an increase in the prevalence of single base deletions that were reported in all samples, both transfected with ZFN or controls (FIG. 7).

From these results highest level of ZFN activity at a FAD2 target site, as determined by the greater activity of NHEJ, was identified at locus E. The ZFNs which were encoded on plasmid pDAB104010 (i.e., ZFN24828 and 24829) were selected for in planta targeting of an Engineered Transgene Integration Platform (ETIP) given its characteristics of significant genomic DNA cleavage activity and minimal non-target activity.

### Example 4

### DNA Constructs for Engineered Transgene Integration Platform (ETIP) Canola Plant Lines

The plasmid vector constructs described below were built using methods and techniques commonly known by one with skill in the art. The application of specific reagents and techniques described within this paragraph are readily known by those with skill in the art, and could be readily interchanged with other reagents and techniques to achieve the desired purpose of building plasmid vector constructs. The restriction endonucleases were obtained from New England BioLabs (NEB; Ipswich, Mass.). Ligations were completed with T4 DNA Ligase (Invitrogen, Carlsbad, Calif.). Gateway reactions were performed using GATEWAY® LR CLONASE® enzyme mix (Invitrogen) for assembling one entry vector into a single destination vector. IN-FUSION™ reactions were performed using IN-FUSION™ Advantage Technology (Clontech, Mountain View, Calif.) for assembling one entry vector into a single destination vector Plasmid preparations were performed using NUCLEOSPIN® Plasmid Kit (Macherey-Nagel Inc., Bethlehem, Pa.) or the Plasmid Midi Kit® (Qiagen) following the instructions of the suppliers. DNA fragments were isolated using QIAquick Gel Extraction Kit™ (Qiagen) after agarose Tris-acetate gel electrophoresis. Colonies of all assembled plasmids were initially screened by restriction digestion of miniprep DNA. Plasmid DNA of selected clones was sequenced by a commercial sequencing vendor (Eurofins MWG Operon, Huntsville, Ala.). Sequence data were assembled and analyzed using the SEQUENCHER™ software (Gene Codes Corp., Ann Arbor, Mich.).

Direct-Delivery Vectors for Precision Integration of ETIP in the Fad2A Locus of Canola

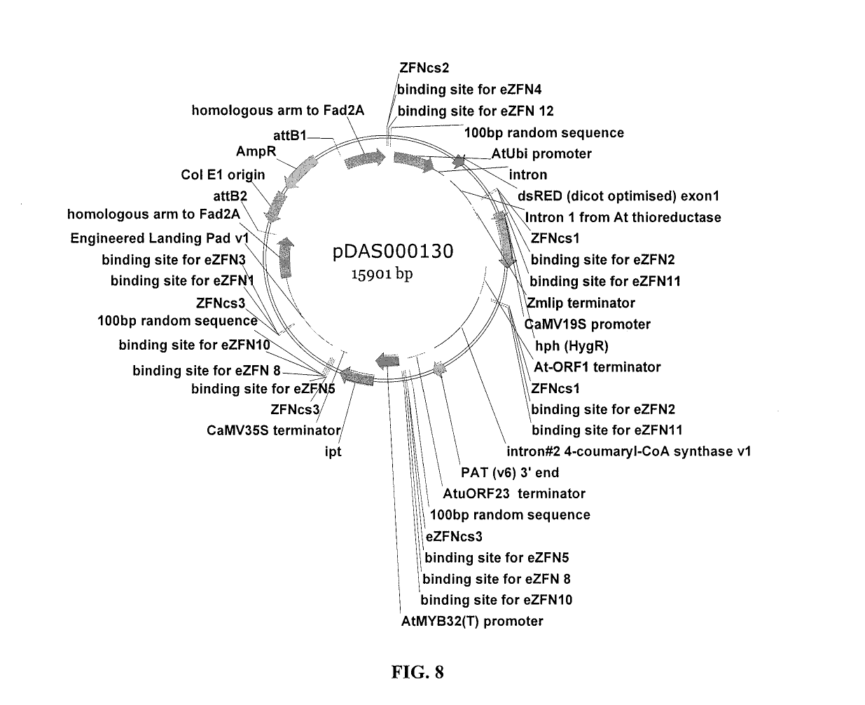

Standard cloning methods were used in the construction of the ETIP-containing vectors pDAS000130 (FIG. 8, T-strand insert as SEQ ID NO:141), for specific integration into the FAD2A gene of B. napus. This construct has been designed to be delivered into canola protoplasts with the Zinc Finger Nuclease construct pDAB 1004010. The Zinc Finger Nuclease Construct will cleave the FAD2A locus and then the pDAS000130 construct will integrated within the canola genome via a homology directed repair mechanism. The ETIP consists of four expression cassettes (two incomplete) separated by additional ZFN recognition sequences and an Engineered Landing Pad (ELP) containing another ZFN recognition sequences. The additional ZFN recognition sequences are unique and have been designed to be targeted for the introduction of polynucleotide sequences within the ETIP and ELP transgene insertions. Similarly, the ZFN recognition sequences can be utilized for excision of polynucleotide sequences. The first gene expression cassette was an incomplete dsRED expression cassette and contained the promoter, 5′ untranslated region and intron from the Arabidopsis thaliana Polyubiquitin 10 (AtUbi promoter) gene (Callis, et al., (1990) J. Biol. Chem., 265: 12486-12493) followed by 210 bp of a dsRed gene from the reef coral Discosoma sp. (Clontech, Mountain View, Calif.) codon-optimized for expression in dicot plants (ds RED (dicot optimized)exon 1) followed by an intron from the Arabidopsis thaliana thioreductase-like gene (Intron 1 from At thioreductase: Accession No: NC_{—}00374) and the 3′ untranslated region comprising the transcriptional terminator and polyadenylation site of the Zea mays Viviparous-1 (Vp1) gene (Zmlip terminator: Paek et al., (1998) Molecules and Cells, 8(3): 336-342). The second expression cassette contained the 19S promoter including 5′ UTR from cauliflower mosaic virus (CaMV 19S: Cook and Penon (1990) Plant Molecular Biology 14(3): 391-405) followed by the hph gene from E. coli, codon-optimized for expression in dicots (hph(HygR): Kaster et al., (1983) Nucleic Acids Research 11(19): 6895-6911) and the 3′UTR comprising the transcriptional terminator and polyadenylation site of open reading frame 1 of A. tumefaciens pTil5955 (At-ORF1 terminator: Barker et al., (1983) Plant Molecular Biology 2(6): 335-50). The third expression cassette was an incomplete PAT expression cassette and contained the first intron from Arabidopsis 4-coumaryl-CoA synthase (intron#2 4-coumaryl-CoA synthase v: Accession No: At3g21320/NC003074) followed by the last 256 bp of a synthetic, plant-optimized version of phosphinothricin acetyl transferase gene, isolated from Streptomyces viridochromogenes, which encodes a protein that confers resistance to inhibitors of glutamine synthetase comprising phosphinothricin, glufosinate, and bialaphos (PAT(v6) 3′ end: Wohlleben et al., (1988) Gene 70(1): 25-37). This cassette was terminated with the 3′ UTR comprising the transcriptional terminator and polyadenylation sites of open reading frame 23 of A. tumefaciens pTi15955 (AtuORF23 terminator: Barker et al., (1983) Plant Molecular Biology 2(6): 335-50). The fourth Expression Cassette was the ipt gene cassette and contained a 588 bp truncated version of the promoter and 5′ UTR from the Arabidopsis DNA-binding protein MYB32 gene (U26933) (AtMYB32(T) promoter: Li et al., (1999) Plant Physiology 121: 313) followed by the isopentyl transferase (ipt) gene from A. tumefaciens and the 35S terminator comprising the transcriptional terminator and polyadenylation sites from cauliflower mosaic virus (CaMV 35S terminator: Chenault et al., (1993) Plant Physiology 101 (4): 1395-1396). For delivery to FAD2A, each end of the ETIP sequence was flanked by 1 kb of FAD2A genomic sequence from either side of the location of the double-stranded break induced by delivery of the ZFN encoded in pDAB104010 to the FAD2A gene of B. napus.

The ETIP sequence was synthesized by a commercial gene synthesis vendor (GeneArt, Life Technologies). The 1 kb segments of FAD2A genome sequence were amplified from genomic DNA purified from leaf tissue of B. napus DH 12075 using a Qiagen DNEASY® plant mini kit (Qiagen, Hilden) following instructions supplied by the manufacturer. The 1 kb FAD2A sequences were ligated into the ETIP vector using T4 ligase (NEB, Ipswich, Mass.). Colonies of all assembled plasmids were initially screened by restriction digestion of miniprep DNA. Restriction endonucleases were obtained from New England BioLabs (NEB, Ipswich, Mass.) and Promega (Promega Corporation, WI). Plasmid preparations were performed using the QIAPREP SPIN MINIPREP® Kit (Qiagen) or the Pure Yield Plasmid MAXIPREP® system (Promega Corporation, WI) following the instructions of the suppliers. Plasmid DNA of selected clones was sequenced using ABI Sanger Sequencing and BIG DYE TERMINATOR® v3.1 cycle sequencing protocol (Applied Biosystems, Life Technologies). Sequence data were assembled and analyzed using the SEQUENCHER™ software (Gene Codes Corp., Ann Arbor, Mich.).

Direct-Delivery Vectors for Precision Integration of ETIP in the Fad 3 Locus of Canola

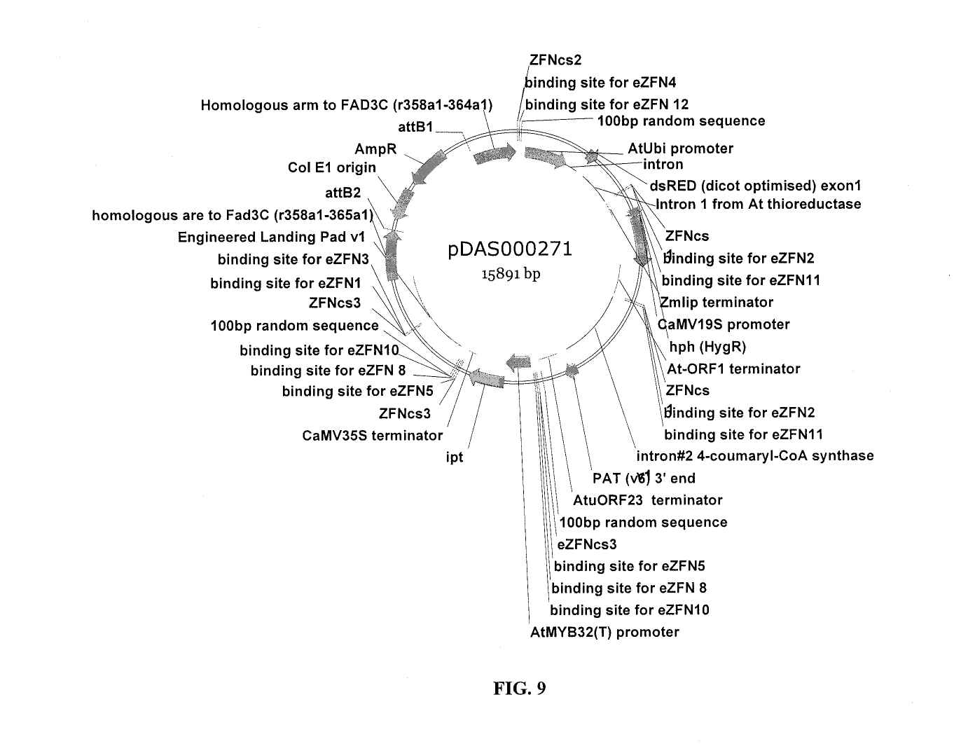

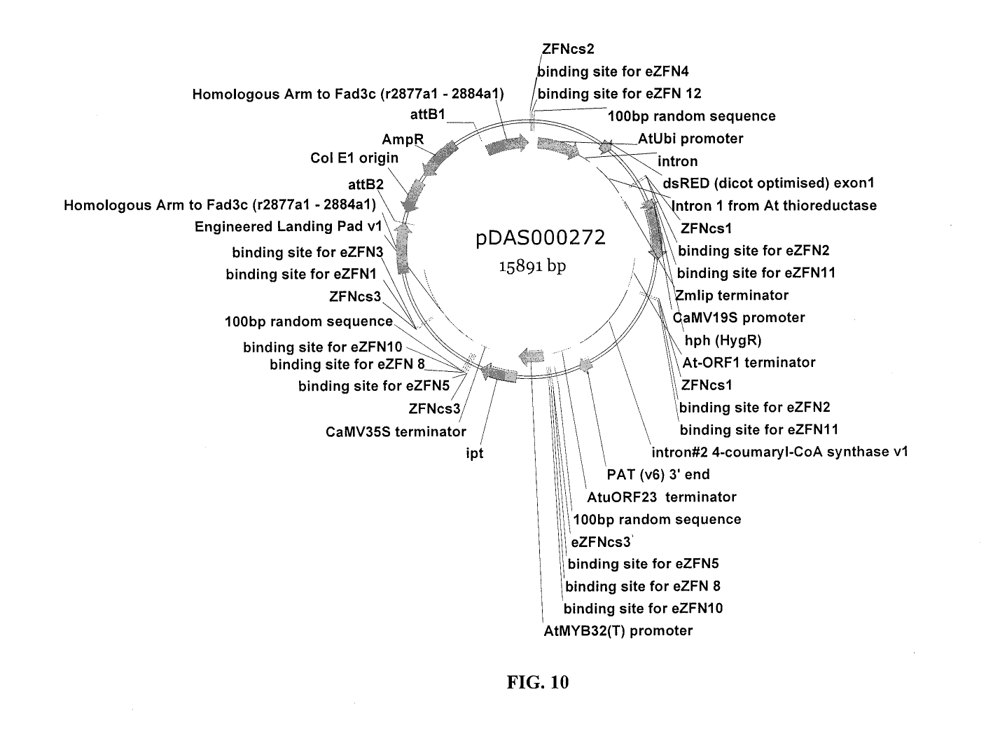

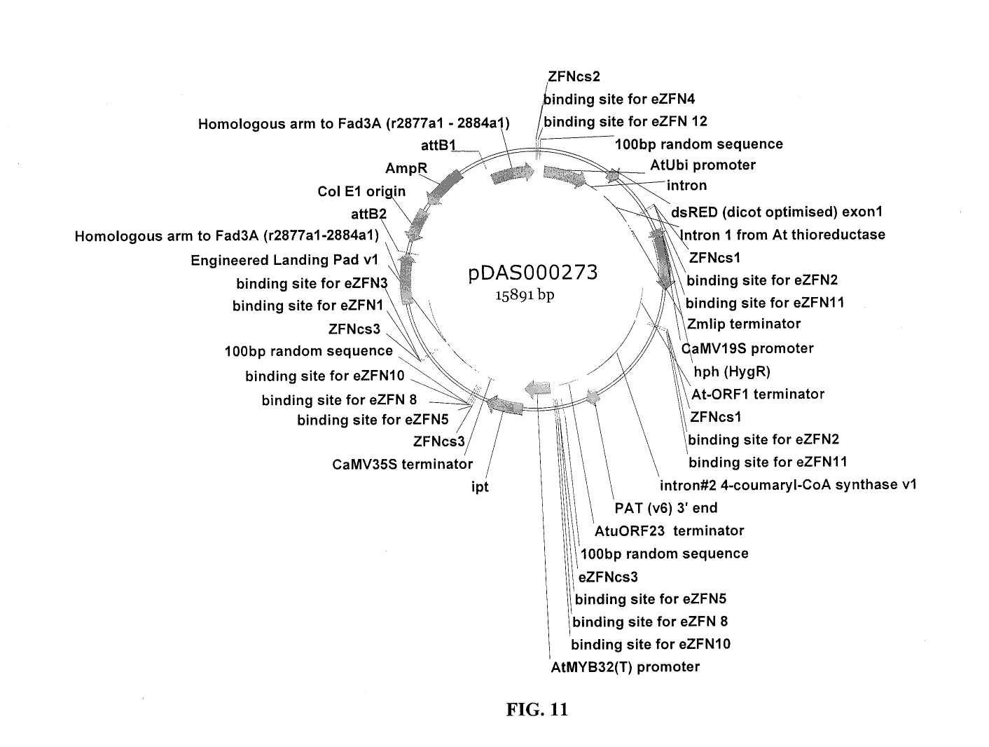

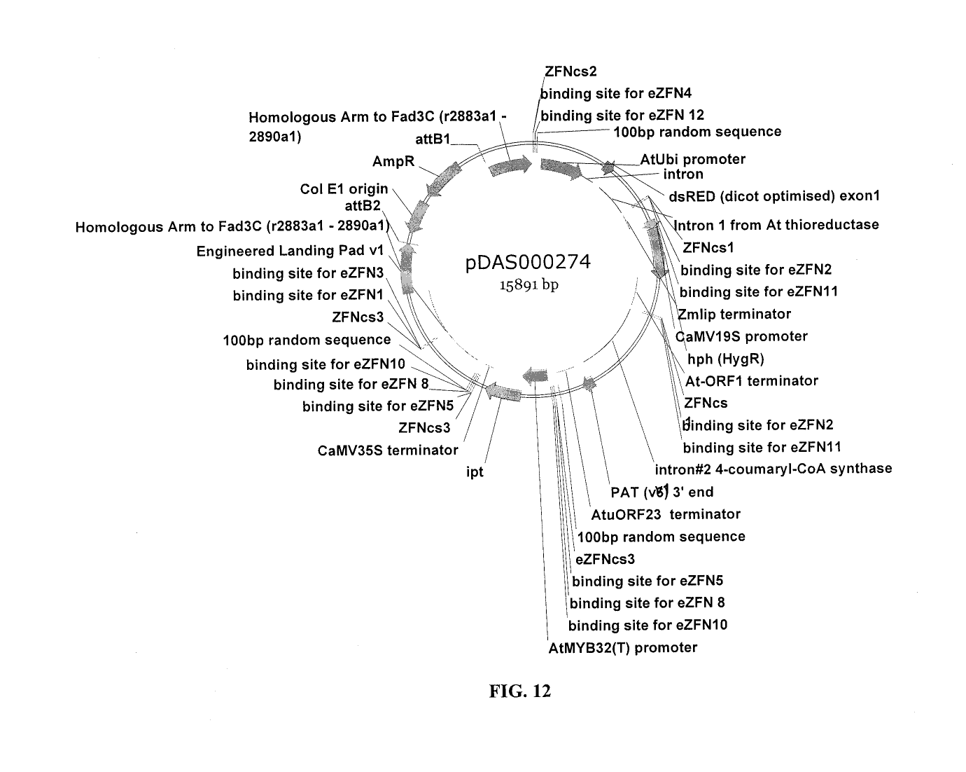

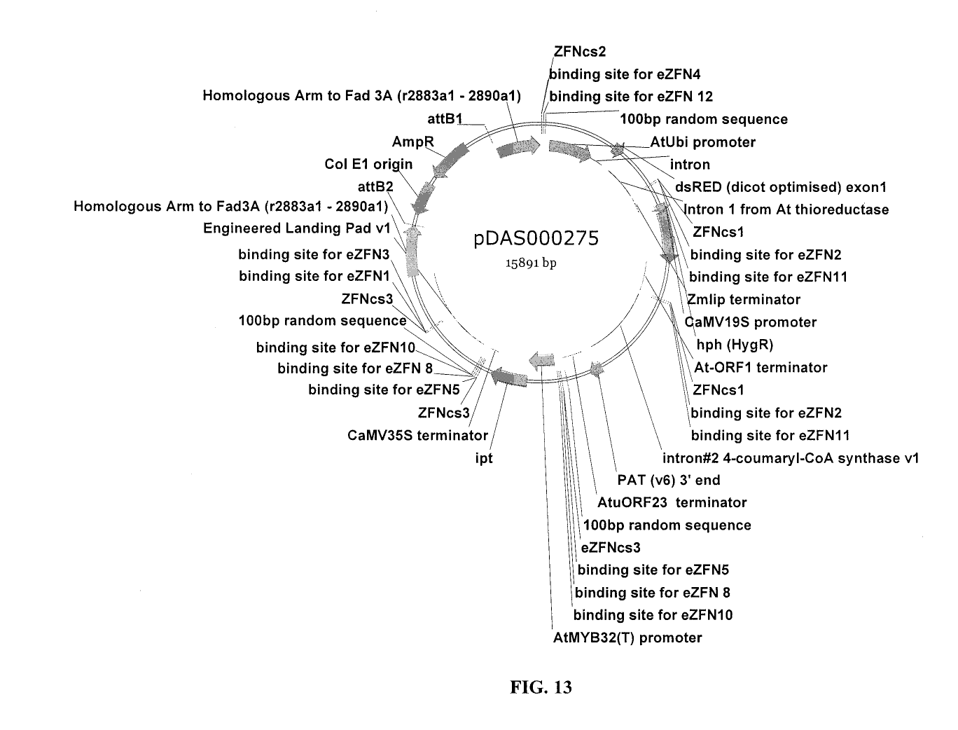

Standard cloning methods were used in the construction of the ETIP-containing vectors pDAS000271 (FIG. 9, T-strand insert as SEQ ID NO:142), pDAS000272 (FIG. 10, T-strand insert as SEQ ID NO:143), pDAS000273 (FIG. 11, T-strand insert as SEQ ID NO:144), pDAS000274 (FIG. 12, T-strand insert as SEQ ID NO:145), and pDAS000275 (FIG. 13, T-strand insert as SEQ ID NO:146) for specific integration into the FAD3A or FAD3C gene locus of B. napus. This construct has been designed to be delivered into canola protoplasts with the Zinc Finger Nuclease construct pDAB107828 or pDAB107829. A specific Zinc Finger Nuclease Construct will cleave the FAD3A locus and then the pDAS000273 or pDAS275 construct will integrate within the canola genome via a homology directed repair mechanism. Likewise, a specific Zinc Finger Nuclease Construct will cleave the FAD3C locus and then the pDAS000271, pDAS000272 or pDAS000274 construct will integrate within the canola genome via a homology directed repair mechanism. The ETIP consists of four expression cassettes (two incomplete) separated by additional ZFN recognition sequences and an Engineered Landing Pad (ELP) containing another ZFN recognition sequences. The additional ZFN recognition sequences are unique and have been designed to be targeted for the introduction of polynucleotide sequences within the ETIP and ELP transgene insertions. Similarly, the ZFN recognition sequences can be utilized for excision of polynucleotide sequences. The first gene expression cassette was an incomplete dsRED expression cassette and contained the promoter, 5′ untranslated region and intron from the Arabidopsis thaliana Polyubiquitin 10 (AtUbi promoter) gene (Callis, et al., (1990) J. Biol. Chem., 265: 12486-12493) followed by 210 bp of a dsRed gene from the reef coral Discosoma sp. (Clontech, Mountain View, Calif.) codon-optimized for expression in dicot plants (ds RED (dicot optimized)exon 1) followed by an intron from the Arabidopsis thaliana thioreductase-like gene (Intron 1 from At thioreductase: Accession No: NC 00374) and the 3′ untranslated region comprising the transcriptional terminator and polyadenylation site of the Zea mays Viviparous-1 (Vp1) gene (Zmlip terminator: Paek et al., (1998) Molecules and Cells, 8(3): 336-342). The second expression cassette contained the 19S promoter including 5′ UTR from cauliflower mosaic virus (CaMV 195: Cook and Penon (1990) Plant Molecular Biology 14(3): 391-405) followed by the hph gene from E. coli, codon-optimized for expression in dicots (hph(HygR): Kaster et al., (1983) Nucleic Acids Research 11(19): 6895-6911) and the 3′UTR comprising the transcriptional terminator and polyadenylation site of open reading frame 1 of A. tumefaciens pTi15955 (At-ORF1 terminator: Barker et al., (1983) Plant Molecular Biology 2(6): 335-50). The third expression cassette was an incomplete PAT expression cassette and contained the first intron from Arabidopsis 4-coumaryl-CoA synthase (intron#2 4-coumaryl-CoA synthase v: Accession No: At3g21320/NC003074) followed by the last 256 bp of a synthetic, plant-optimized version of phosphinothricin acetyl transferase gene, isolated from Streptomyces viridochromogenes, which encodes a protein that confers resistance to inhibitors of glutamine synthetase comprising phosphinothricin, glufosinate, and bialaphos (PAT(v6) 3′ end: Wohlleben et al., (1988) Gene 70(1): 25-37). This cassette was terminated with the 3′ UTR comprising the transcriptional terminator and polyadenylation sites of open reading frame 23 of A. tumefaciens pTi15955 (AtuORF23 terminator: Barker et al., (1983) Plant Molecular Biology 2(6): 335-50). The fourth Expression Cassette was the ipt gene cassette and contained a 588 bp truncated version of the promoter and 5′ UTR from the Arabidopsis DNA-binding protein MYB32 gene (U26933) (AtMYB32(T) promoter: Li et al., (1999) Plant Physiology 121: 313) followed by the isopentyl transferase (ipt) gene from A. tumefaciens and the 35S terminator comprising the transcriptional terminator and polyadenylation sites from cauliflower mosaic virus (CaMV 35S terminator: Chenault et al., (1993) Plant Physiology 101 (4): 1395-1396). For delivery to FAD3A or FAD3C, each end of the ETIP sequence was flanked by 1 kb of FAD3A or FAD3C genomic sequence from either side of the location of the double-stranded break induced by delivery of the ZFN encoded in FAD3A or FAD3c gene of B. napus.

The ETIP sequence was synthesized by a commercial gene synthesis vendor (GeneArt, Life Technologies). The 1 kb segments of FAD3A and FAD3C genome sequence were amplified from genomic DNA purified from leaf tissue of B. napus DH12075 using a Qiagen DNEASY® plant mini kit (Qiagen, Hilden) following instructions supplied by the manufacturer. The 1 kb FAD3A or FAD3C sequences were ligated into the ETIP vector using T4 ligase (NEB, Ipswich, Mass.). Colonies of all assembled plasmids were initially screened by restriction digestion of miniprep DNA. Restriction endonucleases were obtained from New England BioLabs (NEB, Ipswich, Mass.) and Promega (Promega Corporation, WI). Plasmid preparations were performed using the QIAPREP Spin Miniprep® Kit (Qiagen) or the Pure Yield Plasmid MAXIPREP System® (Promega Corporation, WI) following the instructions of the suppliers. Plasmid DNA of selected clones was sequenced using ABI Sanger Sequencing and BIG DYE TERMINATOR® v3.1 cycle sequencing protocol (Applied Biosystems, Life Technologies). Sequence data were assembled and analyzed using the SEQUENCHER™ software (Gene Codes Corp., Ann Arbor, Mich.).

Control Vectors

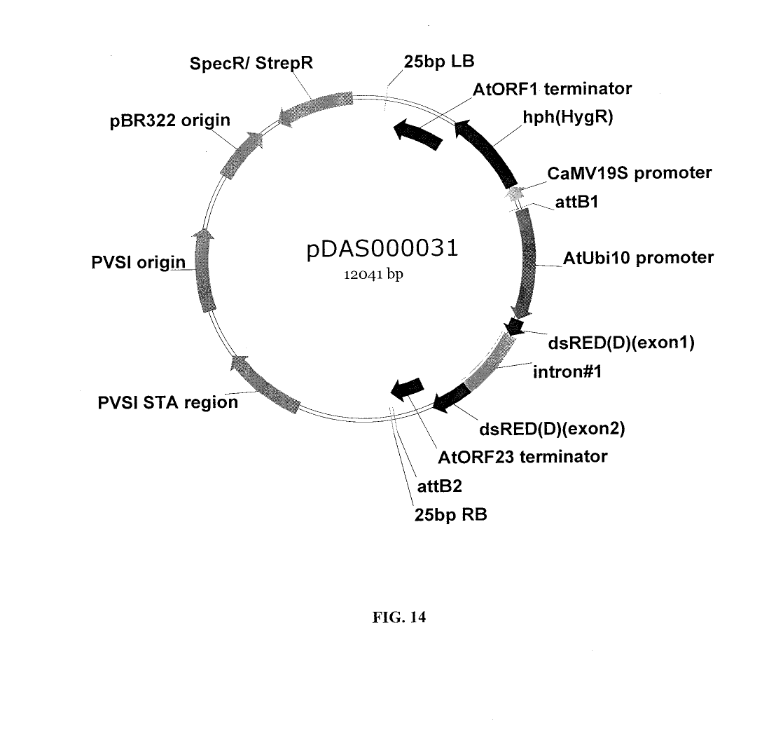

A control vector was used to develop a Fluorescence Activated Cell Sorting (FACS) cell based sorting method. Standard cloning methods were used in the construction of a control vector, pDAS000031 (FIG. 14: T-strand insert as SEQ ID NO:147) consisting of two gene expression cassettes. The first gene expression cassette contained the Cauliflower mosaic virus 19s promoter (CaMV 19S promoter; Shillito, et al., (1985) Bio/Technology 3; 1099-1103):: hygromycin resistance gene (hph(HygR); U.S. Pat. No. 4,727,028):: and the Agrobacterium tumefaciens Open Reading Frame 1 3′ UnTranslated Region (AtORF1 terminator; Huang et al., (1990) J. Bacteriol. 1990 172:1814-1822). The second gene expression cassette contained the Arabidopsis thaliana Ubiquitin 10 promoter (AtUbi10 promoter; Callis, et al., (1990) J. Biol. Chem., 265: 12486-12493):: dsRED (dsRED(D); U.S. Pat. No. 6,852,849) and an intron from Arabidopsis (intron #1; GenBank: AB025639.1) Agrobacterium tumefaciens Open Reading Frame 23 3′ UnTranslated Region (AtORF23 terminator; U.S. Pat. No. 5,428,147) as an in-frame fusion with a trans orientation (e.g., head to head orientation). The plasmid vector was assembled using the IN-FUSION™ Advantage Technology (Clontech, Mountain View, Calif.).

Construction of Binary Vectors for Random Integration of ETIP in Canola

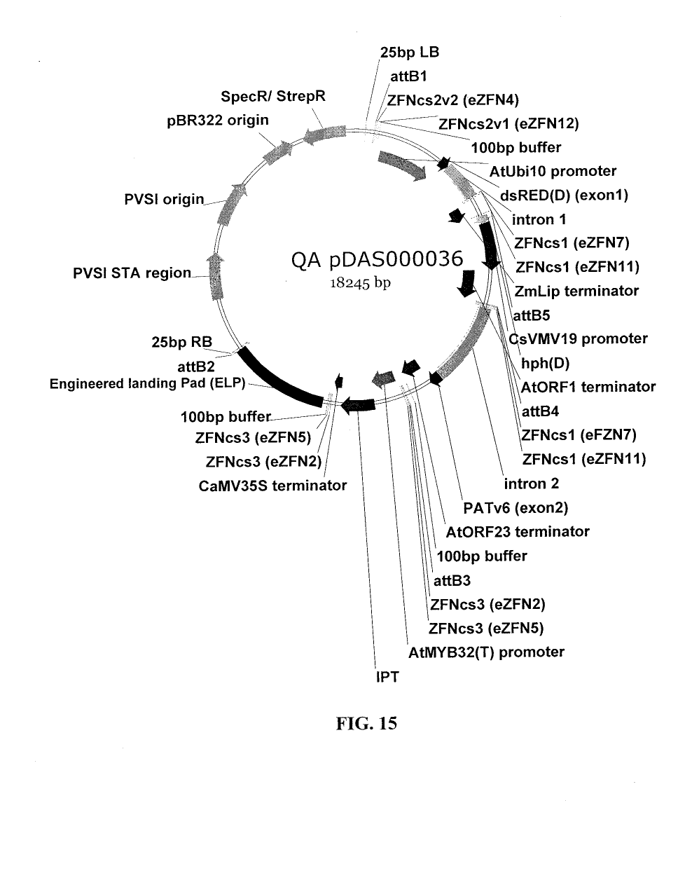

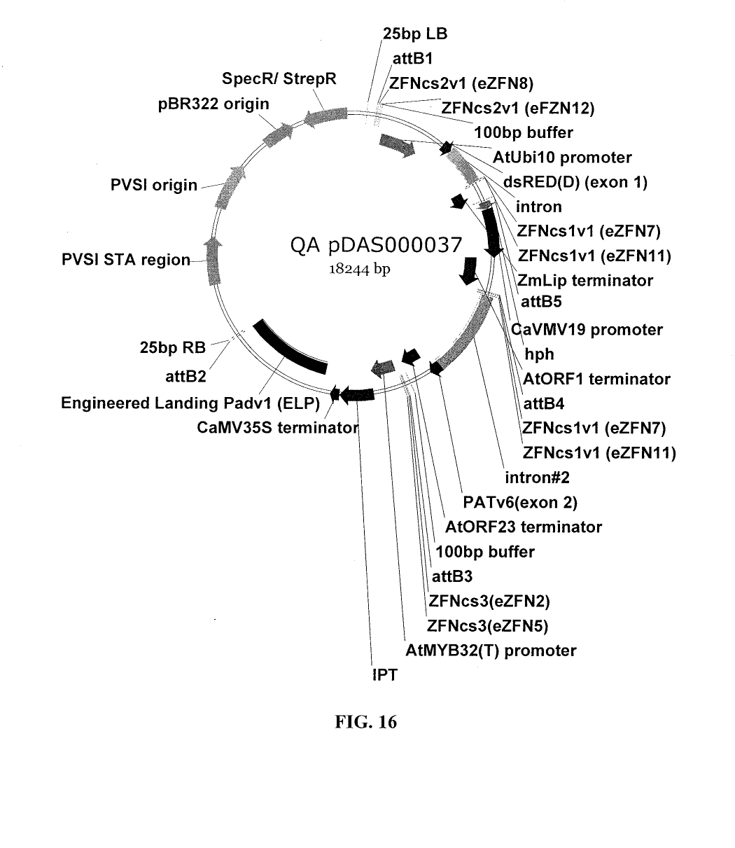

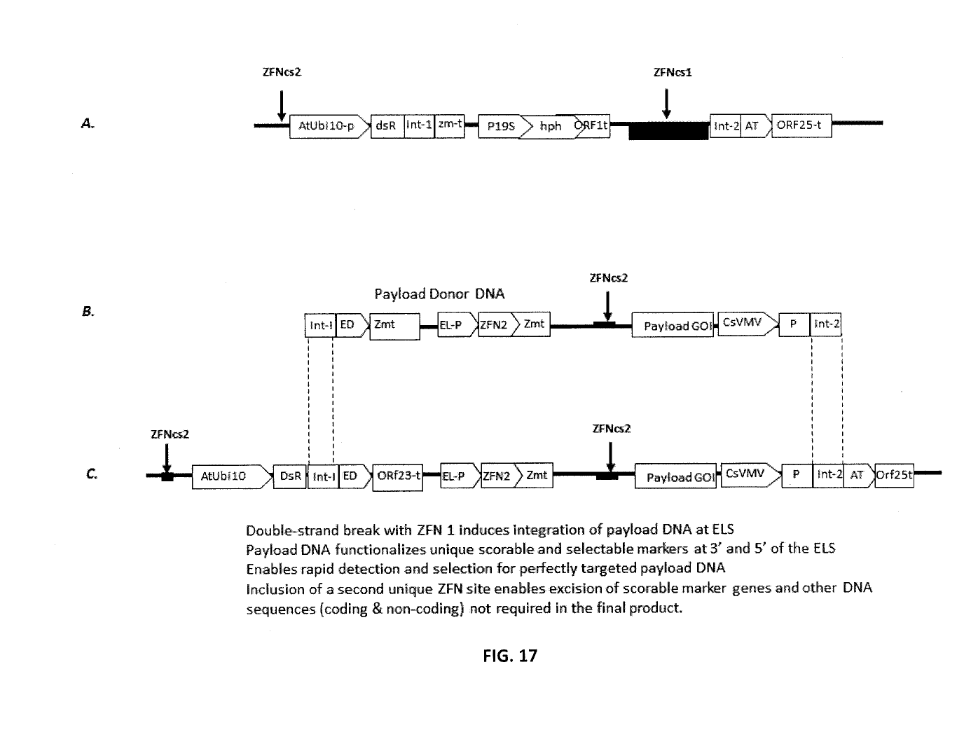

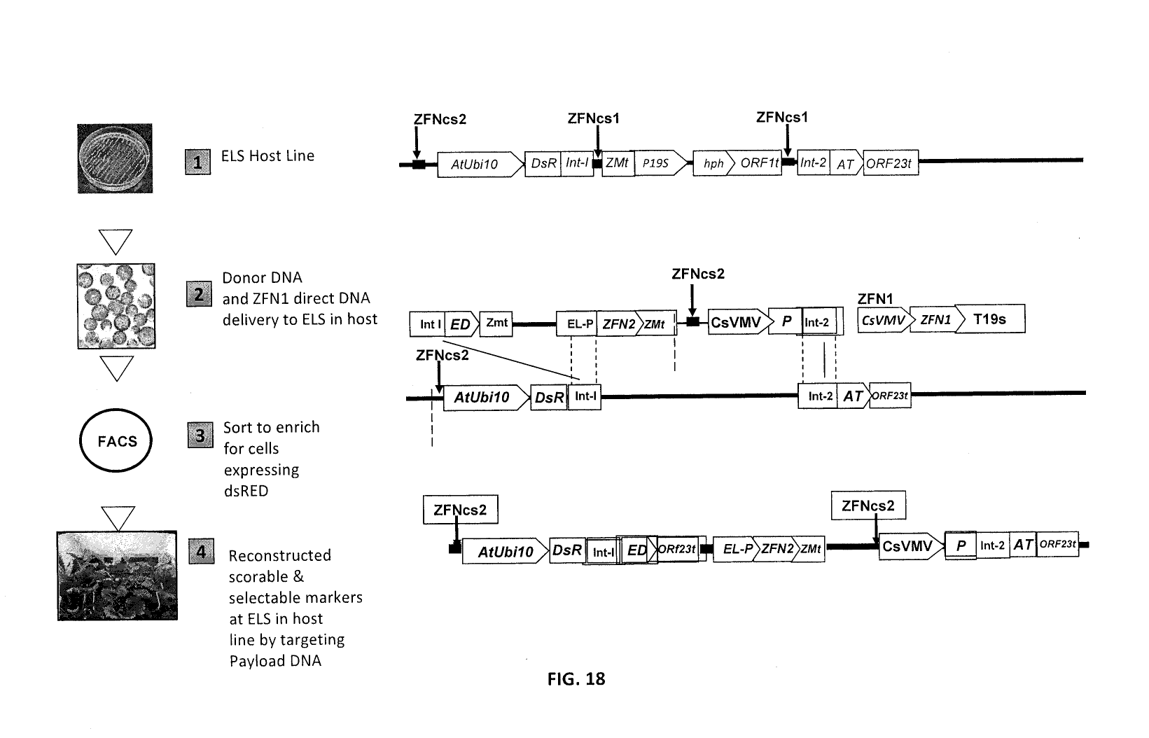

Two binary vectors were constructed for random integration of an ETIP T-Strand sequence within the genome of Brassica napus. Standard cloning methods were used in the construction of the ETIP-containing vectors pDAS000036 (FIG. 15, T-strand insert as SEQ ID NO:148) and pDAS000037 (FIG. 16, T-strand insert as SEQ ID NO:149). The ETIP vectors consist of four expression cassettes (two incomplete expression cassettes) separated by ZFN recognition sequences and an Engineered Landing Pad (ELP) containing further ZFN recognition sequences. The first gene expression cassette was an incomplete dsRED expression cassette and contained the promoter, 5′ untranslated region and intron from the Arabidopsis thaliana polyubiquitin 10 (AtUbi10 promoter) gene (Norris et al., (1993) Plant Molecular Biology, 21(5): 895-906) followed by 210 bp of a dsRed gene from the reef coral Discosoma sp. (Clontech, Mountain View, Calif.) codon-optimized for expression in dicot plants followed by an intron from the Arabidopsis thioreductase-like gene (Accession No: NC_{—}00374) and the 3′ untranslated region (UTR) comprising the transcriptional terminator and polyadenylation site of the Zea mays Viviparous-1 (Vp1) gene (Zm Lip terminator: Paek et al., (1998) Molecules and Cells, 8(3): 336-342). The second expression cassette contained the 19S promoter including 5′ UTR from cauliflower mosaic virus (CsVMV 19 promoter: Cook and Penon (1990) Plant Molecular Biology 14(3): 391-405) followed by the hph gene from E. coli codon-optimized for expression in dicots (hph(D): Kaster et at (1983) Nucleic Acids Research, 11(19): 6895-6911) and the 3′ UTR comprising the transcriptional terminator and polyadenylation site of open reading frame 1 (ORF1) of A. tumefaciens pTi15955 (AtORF1 terminator: Barker et al., (1983) Plant Molecular Biology, 2(6): 335-50). The third expression cassette was an incomplete PAT expression cassette and contained the first intron from Arabidopsis 4-coumaryl-CoA synthase (Accession No: At3g21320 (NC003074)) followed by the last 256 bp of a synthetic, plant-optimized version of phosphinothricin acetyl transferase (PAT) gene, isolated from Streptomyces viridochromogenes, which encodes a protein that confers resistance to inhibitors of glutamine synthetase comprising phosphinothricin, glufosinate, and bialaphos (PATv6(exon2); Wohlleben et al., (1988) Gene, 70(1): 25-37). This cassette was terminated with the 3′ UTR comprising the transcriptional terminator and polyadenylation sites of open reading frames 23 (ORF23) of A. tumefaciens pTi15955 (AtORF23 terminator; Barker et al., (1983) Plant Molecular Biology, 2(6): 335-50). The fourth Expression Cassette was the ipt gene cassette and contained a 588 bp truncated version of the promoter and 5′ UTR from the Arabidopsis DNA-binding protein MYB32 gene (U26933) (AtMYB32(T)promoter; Li et al., (1999) Plant Physiology, 121: 313) followed by the isopentyl transferase (ipt) gene from A. tumefaciens and the 35S terminator comprising the transcriptional terminator and polyadenylation sites from cauliflower mosaic virus (CaMV 35 S terminator; Chenault et al., (1993) Plant Physiology, 101 (4): 1395-1396).

The expression cassettes and ELP were synthesized with Multi-Gateway sites by a commercial gene synthesis vendor (GeneArt, Life Technologies). Entry clones were constructed of each expression cassette and ELP using BP clonase II enzyme mix™ (Invitrogen, Life Technologies) and the pDONR221 vector suite™ (Invitrogen, Life Technologies). The Entry clones were then used in a Multi-Gateway reaction with a Gateway-enabled binary vector using LR Clonase II Plus Enzyme mix™ (Invitrogen, Life Technologies). Colonies of all assembled plasmids were initially screened by restriction digestion of miniprep DNA. Restriction endonucleases were obtained from New England BioLabs (NEB; Ipswich, Mass.) and Promega (Promega Corporation, WI). Plasmid preparations were performed using the QIAprep Spin Miniprep Kit™ (Qiagen, Hilden) or the Pure Yield Plasmid Maxiprep System™ (Promega Corporation, WI) following the instructions of the suppliers. Plasmid DNA of selected clones was sequenced using ABI Sanger Sequencing and Big Dye Terminator v3.1 cycle sequencing protocol™ (Applied Biosystems, Life Technologies). Sequence data were assembled and analyzed using the SEQUENCHER™ software (Gene Codes Corporation, Ann Arbor, Mich.).

### Example 5

### Generation of ETIP Canola Plant Lines

Transformation of Brassica napus

The ETIP constructs (pDAS000036, pDAS000037), the DS-Red control construct (pDAS000031), and the FAD2A, FAD3A, and FAD3C site specific constructs (pDAS000130, and pDAS000271-pDAS000275) and accompanying Zinc Finger Nuclease (pDAB104010, pDAB10728, and pDAB10729) described in Example 4. The binary vectors were transformed into Agrobacterium tumefaciens strain GV3101. PM90. Transformation of Brassica napus protoplast cells was completed using the transfection protocol described in Example 3 with some modification.

The modifications to the protocol included the use of sodium alginate instead of Sea Plaque™ agarose. The transfection experiments in which both the Zinc Finger Nuclease construct and the ETIP construct were co-delivered into Brassica napus protoplast cells were completed at DNA concentrations comprising a 5:1 molar ratio of plasmid DNA. The other ETIP and control plasmid constructs were transformed at concentrations of 30 μg of plasmid DNA. As such, pDAS000130 consisted of a concentration of 27.8 μg of plasmid DNA and pDAB 104010 consisted of a concentration of 2.2 μg of plasmid DNA. The other ETIP and control plasmid constructs were transformed at concentrations of 30 μg of plasmid DNA.

Additional modifications to the protocol included the propagation of whole plants from the transformed protoplast cells in medium containing 1.5 mg/mL of hygromycin. The propagation of whole plants required that the A medium was replaced every two weeks and the growth of the protoplast-derived colonies was monitored. After the protoplast-derived colonies had grown to approximately 2-3 mm in diameter, the colonies were transferred into individual wells of a 12-well COSTAR® plate (Fisher Scientific, St. Louis, Mo.) containing solidified MS morpho medium. The plates were incubated for one to two weeks at 24° C. under continuous dim light until the calli had proliferated to a size of 8-10 mm in diameter. After the protoplast cells had reached a diameter of 1-2 cm in diameter, the protoplast cells were transferred to individual 250 mL culture vessels containing MS morpho medium. The vessels were incubated at 24° C. under 16 h light (20 μMol m⁻² s⁻¹ of Osram L36 W/21 Lumilux white tubes) and 8 h dark conditions. Within one to two weeks, multiple shoots were visible. The shoots were transferred into 250 mL culture vessels containing MS medium after they reached a length of 3-4 cm. The 250 mL culture vessels were incubated at 24° C. under 16 h light (20 μMol m⁻² s⁻¹ of Osram L36 W/21 Lumilux white tubes) and 8 h dark conditions. The shoots were maintained in the culture vessels until they developed into plantlets at which time they were transferred to a greenhouse to grow to maturity.

### Example 6

### Molecular Confirmation of Integration of T-DNAS Containing ETIPS in Canola

Genomic DNA was extracted from leaf tissue of all putative transgenic plants using a DNEASY® 96 Plant DNA extraction kit™ or a DNEASY® Plant Mini Kit™ (Qiagen). The genomic DNA from each plant was analyzed by PCR using primers designed to amplify virC from pTiC58 Forward (SEQ ID NO:150 CGAGAACTTGGCAATTCC) and pTiC58 Reverse (SEQ ID NO:151 TGGCGATTCTGAGATTCC) to test for persistence of A. tumfaciens, primers designed to amplify actin from B. napus; Actin Forward (SEQ ID NO:152 GACTCATCGTACTCTCCCTTCG) and Actin Reverse (SEQ ID NO:153 GACTCATCGTACTCTCCCTTCG) to check the quality of the genomic DNA. Primers were designed to amplify the hph gene; HPH Forward (SEQ ID NO:154 TGTTGGTGGAAGAGGATACG) and HPH Reverse (SEQ ID NO:155 ATCAGCAGCAGCGATAGC) encoded by the ETIP. Plants that did not give a product from virC primers but from which products of the correct size were amplified with primers to actin and hph were classified as transgenic.

A second screen was completed, where gDNA from each transgenic plant was analyzed by PCR using five sets of primers designed to amplify the binary vector outside of the T-DNA region [(1F SEQ ID NO:156 ATGTCCACTGGGTTCGTGCC; 1R SEQ ID NO:157 GAAGGGAACTTATCCGGTCC) (2F SEQ ID NO:158 TGCGCTGCCATTCTCCAAAT; 2R SE ID NO:159 ACCGAGCTCGAATTCAATTC) (3F SEQ ID NO:160 CCTGCATTCGGTTAAACACC; 3R SEQ ID NO:161 CCATCTGGCTTCTGCCTTGC) (4F SEQ ID NO:162 ATTCCGATCCCCAGGGCAGT; 4R SEQ ID NO:163 GCCAACGTTGCAGCCTTGCT) (5F SEQ ID NO:164 GCCCTGGGATGTTGTTAAGT; 5R SEQ ID NO:165 GTAACTTAGGACTTGTGCGA)]. Plants from which PCR products of the correct and expected size were amplified with primer sets 3 and 4 were considered to have backbone integration.

DNA from plants with no backbone integration was purified from 20 g of leaf tissue using a modified CTAB method (Maguire et al., (1994) Plant Molecular Biology Reporter, 12(2): 106-109). The isolated gDNA was digested with several restriction enzymes and 10 μg of gDNA was separated by electrophoresis on an agarose gel and transferred to membrane using a standard Southern blotting protocol. Membranes were probed using the DIG Easy Hyb System™ (Roche, South San Francisco, Calif.) following the manufacturer's instructions. Probes to each expression cassette to the ELP and to an endogenous control gene, actin, were amplified from the ETIP construct using the following primers: (IPT-F SEQ ID NO:166 TCTCTACCTTGATGATCGG; IPT-R SEQ ID NO:167 AACATCTGCTTAACTCTGGC; dsRED-F SEQ ID NO:168 ATGGCTTCATCTGAGAACG; dsRED-R SEQ ID NO:169 TTCCGTATTGGAATTGAGG; PAT-F SEQ ID NO:170 TTGCTTAAGTCTATGGAGGCG; PAT-R SEQ ID NO:171 TGGGTAACTGGCCTAACTGG; ELP-F SEQ ID NO:172 ATGATATGTAGACATAGTGGG; ELP-R SEQ ID NO:173 AGGGTGTAAGGTACTAGCC; Hph-F SEQ ID NO:174 TGTTGGTGGAAGAGGATACG; Hph-R SEQ ID NO:175 ATCAGCAGCAGCGATAGC; actin-F SEQ ID NO:176 GTGGAGAAGAACTACGAGCTACCC; actin-R SEQ ID NO:177 GACTCATCGTACTCTCCCTTCG).

The ETIP sequence was amplified and sequenced from all plants containing only a single copy of the ETIP. The sequence of each T-DNA insert was analyzed by direct sequencing of PCR products using the AB13730xI™ (Applied Biosystems, Life Technologies). The T-DNA insert was amplified from genomic DNA, using Phusion Hot Start II Polymerase™ (Finnzymes, Thermo Fisher Scientific). The amplification reactions of the T-DNA were completed with multiple primer pairs to amplify overlapping sequences of approximately 2 Kbp in length. Each PCR product was sequenced with multiple primers to ensure complete coverage. The PCR reactions were treated with shrimp alkaline phosphatase and exonuclease I (Applied Biosystems, Life Technologies) to inactivate excess primer prior to the sequencing PCR reaction. The sequences flanking the T-DNA insert of each single copy ETIP line were identified by digestion of purified genomic DNA with eight restriction endonucleases followed by ligation of double-stranded adapters specific for the overhangs created by the restriction endonucleases. Following this ligation step a PCR was performed with a biotinylated primer to either the 3′ or 5′ end of the ETIP and a primer to each adapter. The PCR products were captures and cleaned on Ampure Solid Phase Reversible Immobilization (SPRI) beads™ (Agencourt Bioscience Corporation, Beckman Coulter Company). A nested PCR was performed and all products were sequenced using ABI Sanger Sequencing and Big Dye Terminator v3.1 cycle™ sequencing protocol (Applied Biosystems, Life Technologies). Sequence data were assembled and analyzed using the SEQUENCHER™ software (Gene Codes Corp., Ann Arbor, Mich.).

Southern Blot Analysis

Specific restriction enzymes were selected to digest gDNA samples prior to Southern probing. The putative transgenic plants were analyzed by digesting the genomic DNA with EcoRI and SwaI. Next, the digested gDNA and uncut gDNA samples were probed with either polynucleotide fragments comprising PATv6, IPT or ELP gene elements as these polynucleotide probe fragments enabled differentiation of multiple inserts in EcoRI digests as well as in the SwaI digests. Identified single copy transgenic plant lines were then further analyzed with all six probes to identify the presence of all essential elements of the inserted vector.

Accordingly, 67 independent events transformed with ETIP-pDAS000036 were sampled and tested for the presence of the transgene (hph), and the presence of vector backbone. Of the 67 plants tested, 47 were found to have the transgene integrated within the genome. From the 47 transgenic plants, 17 of the plants were found to contain vector backbone (Table 14). The remaining 30 plants that contained no significant portion of vector backbone (absence of Ori or SpecR) were sampled for Southern analysis. As a general rule, the plants were screened initially with the IPT probe, and plant lines identified as putative single copy lines were further tested with probes comprising the dsRED, PAT, ELP and hph gene elements in order to confirm the presence of the whole cassette.

Likewise, 52 independent events transformed with ETIP-pDAS000037 and surviving in soil were sampled and tested for the presence of the transgene (hph), and the presence of vector backbone. Of the 52 plants tested, 48 were found to have the transgene integrated within the genome. From the 48 transgenic plants, 23 of the plants were found to contain vector backbone as well and 3 plants were not tested (Table 14). The remaining 22 plants that contained no significant portion of vector backbone (absence of Ori or SpecR) were sampled for Southern analysis. These transgenic plants were initially screened with the IPT probe, and the plant lines were identified as putative single copy lines, and were further tested with the dsRED, PAT, ELP, hph and actin probes in order to confirm results. Once the identification of 5 independent single copy lines were obtained, Southern analysis was terminated on the remaining plants. In total, 11 ETIP-pDAS000037 lines underwent Southern analysis.

TABLE 14
Summary of +/− transgene and +/− vector backbone PCRs results
Confirmation of transgene - Endpoint PCR

| | Independent | | | Independent |
|---|---|---|---|---|
| | Events | Independent | Independent | Events |
| | Surviving | Events | Events | Positive for |
| | in Soil | Sampled | Tested | Transgene |
| pDAS000036 | 67 | 67 | 67 | 47 |
| pDAS000037 | 52 | 52 | 52 | 48 |

Presence of Backbone - Endpoint PCR

| | Independent | Independent Events with |
|---|---|---|
| | Events Tested | no Ori or Spec^{R} |
| pDAS000036 | 47 | 30 |
| pDAS000037 | 48 | 22 |

Results of ETIP Transgenic Canola Transformed with PDAS000036 AND PDAS000037

The transgenic Brassica napus events which were produced via transformation of pDAS000036 and pDAS000037 resulted in the production of single copy, full length T-strand insertions. Three to four events for each plant were fully characterized, and were putatively mapped to specific chromosomes within the Brassica napus genome. Although a few single base-pair rearrangements occurred during the T-strand integration, the selected events contained full length expression cassettes which are capable of driving robust expression of the transgene. The selected T₀ events were grown to the T₁ stage of development. The T₁ were res-screened using the above described PCR assays to determine the zygosity of the integrated T-strand. Screened events were categorized as homozygous, hemizygous, or null.

The ETIP sequence was amplified and sequenced from all transgenic events containing only a single copy of the integrated ETIP sequence. The sequence of each T-DNA insert was analyzed by direct sequencing of PCR products. The T-DNA insert was amplified from genomic DNA, using Phusion Hot Start II Polymerase™ (Finnzymes, Thermo Fisher Scientific). Next, the T-DNA was amplified with multiple primer pairs to amplify overlapping sequences of approximately 2 Kb in length. Each PCR product was sequenced with multiple primers to ensure complete coverage. The PCR reactions were treated with Shrimp Alkaline Phosphotase and Exonuclease I (Applied Biosystems, Life Technologies) to inactivate excess primer prior to the sequencing PCR reaction.

The sequences flanking the T-DNA insert of each single copy ETIP line was identified by digestion of purified genomic DNA with eight restriction endonucleases followed by ligation of double-stranded adapters specific for the overhangs created by the restriction endonucleases. Following this step a PCR reaction was performed with a biotinylated primer to either the 3′ or 5′ end of the ETIP and a primer to each adapter. The PCR products were captured and cleaned on Ampure Solid Phase Reversible Immobilization™ (SPRI) beads (Agencourt Bioscience Corporation, Beckman Coulter Company). A nested PCR was performed and all products were sequenced using ABI Sanger Sequencing and Big Dye Terminator v3.1 cycle sequencing protocol (Applied Biosystems, Life Technologies). Sequence data were assembled and analyzed using the SEQUENCHER™ software (Gene Codes Corp., Ann Arbor, Mich.). Eight ETIP lines were identified and selected for flanking sequence analysis (Table 15). The left and right flanking sequences (also described as border or junction sequences) are provided as SEQ ID NO:431-SEQ ID NO:446, the underlined sequences indicated plasmid vector, the non-underlined sequences indicate genomic flanking sequence.

TABLE 15
Details of single copy events used in flanking sequence studies

| | | | Left Hand | Right |
|---|---|---|---|---|
| Plasmid | | | Border SEQ | Hand SEQ |
| Description | Event name | Barcode | ID NO: | ID NO: |
| pDAS000036 | em02-5788-1-1 | 228688 | 431 | 432 |
| | ad58-5784-2-1 | 232502 | 433 | 434 |
| | ad58-5898-10-1 | 237143 | 435 | 436 |
| pDAS000037 | lf31-6139-2-3 | 234576 | 437 | 438 |
| | bm56-6315-1-1 | 234703 | 439 | 440 |
| | ad58-6372-1-1 | 240653 | 441 | 442 |
| | ad58-6620-4-1 | 242268 | 443 | 444 |
| | ad58-6620-17-1 | 242293 | 445 | 446 |

```
pDAS000036 Event details: em02-5788-1-1
Left border flanking
(SEQ ID NO: 431)
                    TCGAGATTGTGCTGAAGTAAACCATTTTACTTCAAATCTA
TTTTTAACTATTTACTTTTATTAAGGAGAGAAACTTTGCTGATTAATTCA
AATTAGTGATCATTAAGATTCCAAAGATTCCGATTTAGAAAAGTCAAAGA
TTCAAAGAACAAGTCTAGGTCCTCATGGCTCATGTTGCATCCGATTCACC
ATCCACTCATCTTTCATATCTTCCTCCACTGTCTCTCTAGAAACAACTCA
TTTAATTTAGAAAACTCCTTTTTCAATTTAGAAATATTAAAGTTTATCAC
AATGTATCAATTAAATATTATCCGATGACTCATTCATAGTCAGGACCTTG
CTGTCTGTGTCGTCCGTAATTATTATTTCAATACAAAACAAATATATGTT
CACTCAGAAAATTACGGCGCAATCATCTAATTTTGTGGACCAAAATAAAT
AGCGTAGCTTCGAGATTTCAAAGTTGTGTTCAAATTTAATTTTGATTTCC
GTTCCTCGTATACTCTTTTATGTATAGAAAATAATAATATCCACTACTAG
TAGTTGATAACTACATTACATATATTAAAATTATGATGTCACATTGCGGA
CGTTTTTAATGTACTGAATTAACGCCGAATTGAATTCGAGCTCGGTACCC
GGGGATCCTCTAGAGTCGACCTGCAGGCATGCAAGCTTAGCTTGAGCTTG
GATC
em02-5788-1-1 Left border flanking
(SEQ ID NO: 432)
                    CATAACCACCATCTCAAACAATAGAACTTCCTAAGTGAAG
CAATGACTTCAAATCTACTTGAAGGCATGGAGTATAAGCCATGTTCCTTT
CAGAGGGGACTGTACTTCTGTAGATTACTTTCCCTCATTAACCAGATCTG
GCCGGCCTACCCAGCTTTCTTGTACACATAGCGACCGAGCTCGAGCCGAA
GTTCGGTCGCTGTTTCACTGTTTGGGAAAGCATCAGTAACGCAGAAGACA
TAATTAAAAATTAATTATATATGGTAGTTTTTCTAGATTCTCCACTATAC
CTCATTGTGATTGAAAAACAACTATATATATATATATATATGTATTTAAA
ATTAAGAAATCATTAAATCGTACCATAATGCAGAAAACTTTATAAGTTCC
TATTCTTTTGTCAAGATGAGTAGATGACACATCATGTACCACATAACATA
ACCATAATGGTGCGATACCATACCAAAACTTAATTTAGAAACTAATTAAA
ATTTTGGTAGTTTAAGATTCCTCCTAATGGTTGTCAAAAAAAAAAAAAGA
TTCCTCCTAATACATGGTAGAATATATTTTTGAGTTAAAATGAAAGTAAA
ATCTTCAAATTAGTCATAAGGAAATTCTAAAAAACATCGACTTTCTTTAT
AAAGATCCCATTGTAATTTTAGATGATTAATTTTATCCCAATCCAATTAA
GAATTGTACACATCGGCCTCTATATATATCAAACACCTAAAA
pDAS000036 Event details: ad58-5784-2-1
Left border flanking
(SEQ ID NO: 433)
                    CGATTTGCAGCTATAATCAATCACACCTTATCGTTCTTTC
AAAGAAAAATCGAAAGTTGTAAACTTTATCAGCCTGTGTAGTGATTATTT
CAATTTGATAAAGAAAAAAAAAGGCTTAGCTTTATTTGGTTTTTTGTTAC
AATCTTGATTAATTTTAGATTAGCACTCTGATTCTAGCGGAACATGAGAG
TGGTTCCATCAAACCTCAGACAGTGAGCACAGTGGTTGCAGCAAACCATT
TGGGTGAGAGCTCTTCAGTTTCTTTGCTATTAGCTGGTTCTGGCTCTTCT
CTTCAAGAAGCTGCTTCTCAAGCTGCCTCTTGTCATCCTTCTGTTTCCGA
GGTAACTAACTACATTCTTCATTTGTCCTTTTTTCTTGTGGGTCTTAAAT
GTTYGTGCTTTTCTTTATAGGTACTTGTTGCTGATTCAGATAGATTTGAG
TACCCTTTAGCTGAACCTTGGGCTAAACTGGTTGACTTTGTTCGCCAACA
AAGAGATTACTCTCACATCCTTGCCTTCCTCTAGCTCATTTGGCAAGAAC
ATACTTCCTCTAGACAACTTAATAACACATTGCGGACGTTTTTAATGTAC
TGAATTAACGCCGAATTGAATTCGAGCTCGGTACCCGGGGATCCTCTAGA
GTC
ad58-5784-2-1 Right border flanking
(SEQ ID NO: 434)
                    CCTGTCATAACCACCATCTCAAACAATAGAACTTCCTAAG
TGAAGCAATGACTTCAAATCTACTTGAAGGCATGGAGTATAAGCCATGTT
CCTTTCAGAGGGGACTGTACTTCTGTAGATTACTTTCCCTCATTAACCAG
ATCTGGCCGGCCTACCCAGCTTTCTTGTACAAAGTGGTGATAAACTATCG
CCGGCCTACCTCGCGTTGCTGCTCTTTTAGATGTCTCTCCTGTTACTGAT
GTTGTCAAAATCTTAGGATCCAATGAGTTTATCAGGTATACTTCTATCAT
GTATTGCTTGAGATTTTGGAGTGTTAGTAAAGATTTCAATAAAAGAATTT
TTTCAAACAAAATTTTGGGGGCTTGAAGCTAATGTTTGGAAATATGTAAC
GGAGTTTAAATCTTTTGGCAGGCCTATATATGCGGGAAACGCCTTGTGTA
GAGTTCGCTATACTGGTGCTGGTCCTTGTGTGTTGACTATTAGAACTACA
TCTTTTCCTGTTACCCCAATAACTGAGTCAAAGAAAGCTACTATCTCTCA
GATTGATCTCTCGAAATTCAAAGAAGGTTTGTTTAGTATTATTCTCTTGT
GCATAGCCTTTTTGCTTTTTTTTTTTTTATAAAAAAAGTTGAGTATGCTT
ATTGCCCATTGCA
pDAS000036 Event details: ad58-5898-10-1
Left border flanking
(SEQ ID NO: 435)
                    TAATTTCATTTTCGTCATTTTGGTAAAGTAACAAAAGACA
GAATATTGGTTAGTCGTGTTGGTTAGGAATAAAATAAAGAACGTGGACAT
CGTGGAATAAAAATATTCAGACAAGGAAACTAACAATAAAACAGTAATGA
ACATGGTTCTGAATCTCATCTTTGTGTATCTCCAATGGAATCCACCGCCA
CGAATCAGACTCCTTCTCCAAGCTCCACCGTCGACGATGACAATGGCGAC
GGTGTCTATACCGACGAATTTACCAAACTACCCCCGGACCCGGGGATCCT
CTAGAGTCAACAAATTGACGCTTAGACAACTTAATAACACATTGCGGACG
TTTTTAATGTACTGAATTAACGCCGAATTGAATTCGAGCTCGGTACCCGG
GGATCCTCTAGAGTCGACCTGCAGGCATGCAAGCTTAGCTTGAGCTTGGA
TCAGATTGTC
ad58-5898-10-1 Right border flanking
(SEQ ID NO: 436)
                    CGGCCTACCCAGCTTTCTTGTACAAAGTGGTGATAAACTA
TCAGTGTTTGATTAAAGATAAAATTTGATTTTTCATTACATAATAATCCA
TTAATTTTCACGCACGTGGAACCCATTTGGTGTACTTCCACGTCCTCTAA
GAGAGCACTGACCCACTCATCAAAAAGATACATCTTTATAAGCCCCTTCA
TCGTCACACAGACACACACTTCCCTCTCAATTATTCCATATTCTCCTAAT
TTCTAATTGTTACACCTCAACACATTAGATTCGTACCAAACAAAAACGAT
TAGCTCCCAAGCCTAAGCTTTTATTTCCTTATAATTTTTCTTGGGTTTCT
CTCTATAAAGAATGCAAATGACTGAGAGAGGCCGAGCCATGTGGCACACG
TCCCTAGCCTCGGCATTCCGCACAGCTCTAGCTTGCACAATCGTTGGTGC
GGCTACGCTCTACGGACCCGAGTGGATCCTCCGTTTTGTGGCATTCCCGG
CGTTTTCTTACGTCACGGTCATTCTCATCATTACGGACGCCACGCTAGGC
GACACACTACGTGGCTGCTGGCTAGCCCTTTACGCCACATGTCAGAGCGT
TGCACCGGCTATCATTACACTAAGGCTTATAGGACCAGCTCGGCTCACGG
CCGG
pDAS000037 Event details: 1f31-6139-2-3
Left border flanking
(SEQ ID NO: 437)
                    TTTCTTTCCATCAGTTCTTCGGCACCTTCCTGGCTCTGCG
TCTATCTTTCTTTCCATCCCGGCCCATCTCGTACACATTCCACCCAACTA
CACAAACACGTTATAGTCTTTTACATTATGACCAAATCAACCCTAAAGAT
ACAGCCTTTATAAAAAATATAGGGGTCAAAGCAAAGAAGAGAAAGTTTGC
TTACAGTGTGGAGAAAAGAAGTTGGAAGATGAGGAGTAAGAAGAAGAAGA
GAAGAGAAAGGGTCTTCTGATGAGGAATAGGAGATAAGGTGGAACTGGAA
TGTTTGCCGCTAAATACTTGAAGACAAGAGCTTGATTTTCAAGCTCTTCC
CATTGTGACTCAGTGAAAGGGATCCTAGTCGTCATGAAGAGATAGAGAAA
ATTGATGATGAAGGAGGTCTGATGAAGAGAAGAGAGAGAGAGAGATATTA
CACAAAGAGGGTTGTATTGCAAAATTGAAAGTGTAGAGAGAGTAGTAGGT
AAGTTTTTATTAATAATGTTGTTCACACCTGCAGTCTGCAGAATATTGTG
GTGTGAACAAATTGACGCTTAGACAACTTAATAACACATTGCGGACGTTT
TTAATGTACTGAATTAACGCCGAATTGAATTCGAGCTCGGTACCCGGGGA
TCCTCTAGAGTCGACCTGCAGGCATGCAAGCTTA
1f31-6139-2-3 Right border flanking
(SEQ ID NO: 438)
                    TTCAATCTACTTGAAGGCATGGAGTATAAGCCATGTTCCT
TTCAGAGGGGACTGTACTTCTGTAGATTACTTTCCCTCATTAACCAGATC
TGGCCGGCCTACCCAGCTTTCTTGTACAAAGTGGTGATAAACTATCAGTG
TTTGAACATATATATACGCATAATATTCTCAGAACCCGACCCATTGGTTG
ACTCGGATCAAGATCGACCCGATCCGACCCGGTTAAAAGCACGTCGTCTC
CTTTGGTTCGCCCCTTATATTCGACGAGTGAGTTCGATTGGATCGCTGTT
TGTCATTTCTCACTACCTTAAGAAAAAAAAAAAGGTGCGTCTCTCTCTCA
CCTTTACCGCTCACTTACCTCTCAGATCTGACATCGATTTTCCAAATCTT
C:TCCAGGTACTCTCTCTCCTGGCCGTTGACGGTCCCGTCCCGGCCGTGG
ATCTGATTTCGCCGCGATCTGAGGTCAAGCATGGCGGCAGCTAACGCCCC
CATCACGATGAAGGAGGTCCTAACGGTGAGTCCCGCCTCCATTTTTAGTA
ACATA
pDAS000037 Event details: bm56-6315-1-1
Left border flanking
(SEQ ID NO: 439)
                    TACATCGCGATTCATCCTGGTTTGATTAGAATGACGAGGA
AGTTGTCATATTCCCAAACAGGAAAATTGGGATCGCCTTATTTGAAAGTG
GGATAACTTCTTCATCTTAATTCTTATGAGAATTATTCCACTTCCTGGTG
ATTCTCCACTACTTTTTGTATATAAATACAGCTTCTTACATCGCGATTCA
TCCTGATTTGATTAGAATGACGAGAAAGTTGTCATATTCCCAAACAGGAA
AACTGGGATCACCTGATTTGAAAGTGGGATAACTTCTTCATCCTAATTCT
TATGAGATTTATTCCACTTCCTGGTGATTCTCCACTTCTTTATGTATCCA
AATACATCTTCTTACATCGCGATTCATCCTGGTTTGATTAGAATGACGAG
GAAGTTGTCATATTCCCAAACATGAAAACTGGGAAAGTGGGATTGACGCT
TAGACAACTTAATAACACATTGCGGACGTTTTTAATGTACTGAATTAACG
CCGAATTGAATTCGAGCTCGGTACCCGGGGATCCTCTAGAGTCGACCTGC
AGGCATGCAAGCTTAGCTTGAGCTTGGAT
bm56-6315-1-1 Right border flanking
(SEQ ID NO: 440)
                    CCACCATCTCAAACAATAGAACTTCCTAAGTGAAGCAATG
ACTTCAAATCTACTTGAAGGCATGGAGTATAAGCCATGTTCCTTTCAGAG
GGGACTGTACTTCTGTAGATTACTTTCCCTCATTAACCAGATCTGGCCGG
CCTACCCAGCTTTCTTGTACAAAGTGACGATAAACTATCAGTCTTTCAAA
GCGCATCTATGGCTAGTCATCACGGTTTTTAACTGTTTTACGAAGTCGCG
GAGAGGCTCGTCTTCTCCCTAGGATAAACTGCAGAGGTCGACATCGGAGG
TTTCTCGATCTATGAACATAGAGTACTGTTTGAGAAACTCCGAGGCGAGT
TGGCGGAAACTCCCTATAGAGTTTTTCTTTAGACAAGAGAACCACTCAAG
CGCTGCTTCGCGGAGGTTCTCGACGAAGAGGCGGCATTCGCCGGCATCTC
TTTCTCTATCTTTAAACTTGGCTCTTCCCATCGCGATCTGGAAAGCCTGC
AAGTGTGCCTTAGGATCGGTTGTACCATCGTAAGGAGCCACTTTGACTTT
ATCAGGATCCGAAACCGTAGTTTCGGTGATGCGGGTGGTGAAGGGCGTTT
TTCGAGACTCCTCGAGGAGTAGGGTGATATCGAGTGCAGTACTAGTAGCG
TGATGATTTGGGATTTCACCGCTCTAACCTCCGCAGCCGTTTTCA
pDAS000037 Event details: ad58-6372-1-1
Left border flanking
(SEQ ID NO: 441)
                    TTTTACAGTGTTAGAAGAAGTGGATGAAGCTGAAATTGAA
TTTTCAAACTCGTTCAGCTTGACTAGAGGTGGGAGAGAGTCAAAGCCTCC
CATCAAGTACCAAAACATGGAATAGAAGACAGTCCGAGGGAGAGGAAACC
GTGGCCGACGAGGCCGTGGCTCCTATCATTAACTAGTGTCTTTCTTACTA
TTAATGGTTTATTAAGTCTCAGCCTTTGTTGTTTCATTGGTTTGAGATTC
ACTCATACATGAAACTTGTTTCATTCCAGCTTTTCCAAACTATAAGAATA
TTTCCAATCTTATCTTGTAATAGTTTAAGTTTTAAATTGAAAGCCCTTAG
TTCAAAAAACAAAAAAAAAAAATTAGCCCTTGAATTTATATATAATCACG
ACGGCCATATTTGGCAGCTACACTGATATGTTTTCAATTGGCTGACAGGC
CTTGAGCAGGGTTTGCTGGGTATATTGGTAGGAAGATGTGTTGCGAGGTT
GAAGCCTCATTTAGGCAATATAAACATGATCATTAGCGTTATGTCATTAG
TTATACTTATACGTAGACTAAGTAACCCACTAAAGGTTGCTGATTCCTTT
TGTATCGACTAACACATTGCGGACGTTTTTAATGTACTGAATTAACGCCG
AATTGAATTCGAGCTCGGTACCCGGGGATCCTCTAGAGTCGACCTGCAGG
CATGCAAGCTTAGCTTGAGCTTGGATCAGATTGTCGTTTCCCGC
ad58-6372-1-1 Right border flanking
(SEQ ID NO: 442)
CATGTTCCTTTCAGAGGGGACTGTACTTCTGTAGATTACTTTCCCTCATT
AACCAGATCTGGCCGGCCTACCCAGCTTTCTTGTACAAAGTGGTGATAAA
CTATCAGTGTTTGACTGAATTTTAATTTCTAATTTTTGTAAAAAATTTGT
ATAACCTCAAATTATTAAAAGGCGGATTTTATTAGAATTATAACTAAATT
ATCTATAACTCCAAAATTTTGACAATCAATCATGTCTATATCTTTATTTT
TTTGCTAAATTATCATGTCTATATCTTTTCTTTCTTCCAAACTTACTTGA
GACTAAAAGTCTTTATAAATTTTGATAGGAGTTCCACACACAAACAAAAA
CAAAACAAATATTTTTCATCAAGGGATACTTATTTAACATCACGGATTCA
CAGTTTATTAACAAAAATCCAAACAAAGACTGAAAGACAGAAGATTCAAT
CTAACAATAGTCGGCAAACACCAGTGATTAACTAACGAAATAAATTAACA
AGTGGTCAGATCTTCGGGAAA
pDAS000037 Event details: ad58-6620-4-1
Left border flanking
(SEQ ID NO: 443)
                    TAATTTCATTTTCGTCATTTTGGTAAAGTAACAAAAGACA
GAATATTGGTTAGTCGTGTTGGTTAGGAATAAAATAAAGAACGTGGACAT
CGTGGAATAAAAATATTCAGACAAGGAAACTAACAATAAAACAGTAATGA
ACATGGTTCTGAATCTCATCTTTGTGTATCTCCAATGGAATCCACCGCCA
CGAATCAGACTCCTTCTCCAAGCTCCACCGTCGACGATGACAATGGCGAC
GGTGTCTATACCGACGAATTTACCAAACTACCCCCGGACCCGGGGATCCT
CTAGAGTCAACAAATTGACGCTTAGACAACTTAATAACACATTGCGGACG
TTTTTAATGTACTGAATTAACGCCGAATTGAATTCGAGCTCGGTACCCGG
GGATCCTCTAGAGTCGACCTGCAGGCATGCAAGCTTAGCTTGAGCTTGGA
TCAGATTGTC
ad58-6620-4-1 Right border flanking
(SEQ ID NO: 444)
                    CATAACCACCATCTCAAACAATAGAACTTCCTAAGTGAAG
CAATGACTTCAAATCTACTTGAAGGCATGGAGTATAAGCCATGTTCCTTT
CAGAGGGGACTGTACTTCTGTAGATTACTTTCCCTCATTAACCAGATCTG
GCCGGCCTACCCAGCTTTCTTGTACAAAGTGGTGATAAACTATCAGTGTT
TGAAATAATCGGATATTTAATTTTCTTAGACAGTTCATTAGTAGTTGATC
TTAAACATTCACGTTTTATTTTCTTTTCTTTTCGAATGCTAGACTCTAGT
TTGGTACCCATAGGATTCGAGTTACATGAAGCTATTGACACTGGGAGTGC
TTCAAGATCTGTAAGAGGCAAAGATTCACAAACAGAACGTGATTTCTTGG
ATAGTGATGTGGAGATTGTGATAAGAACCAGCATGAGTATTACTTTTACT
GCCCCTGCTGTGGTGAAGACATCACCAAAACAGTCAAGCTCGTGAAGAAA
TCAGATATTCAACCCGCAAAAAAATCTGACAATGCAAATAAACCTATTGA
CACTAAGAATGGTTCAAGATCCGAAGACAAGAAGACAAAAAATTTGTCCT
GGCTCCCTGCTTATCTCCAGAAGCTGTTTCTTTCTGTTTATGGCCACATC
AAAGACAAAGGTACCCTTTCTTTTGGTGCCTTTGGTGTGGACAGGTGTGA
TTGACTTTGGTTTCTTGGCAGATTCAGGCAAGATAGAGGTTGATTCAAAG
TCAACTAACAATGATCTTGGTACCAATAGTGAGGA
pDAS000037 Event details: ad58-6620-17-1
Left border flanking
(SEQ ID NO: 445)
                    CCGCCTTGAACAACCGCTCCGCCGTTGCTCAGTACATTAT
CGAGGTTACCAAAAAGTTCAATCCTTTATGCTTGTTTTGGCGTGTGATTC
GTTACGAATGAGTAAAATTGATTTGGTTTTTTTCTTTGAACAGCATGGTG
GAGATGTGAATGCGACGGATCATACGGGGCAGACTGCGTTGCATTGGAGT
GCGGTTCGTGGTGCGGTGCAAGTTGCGGAGCTTTTGCTTCAAGAGGGTGC
AAGGGTTGATGCTACGGATATGTATGGATATCAGGTTCTAACCACTTCCT
CTTTCTTTGTGGAGATTGTCTTTTTGTTTCAATGCTAGTCAACTTCTTTC
TTTCTTTCACAAAACTAAGTAGTATTGCTTGTTTTGTTGTCGTTGCATTT
TTTCTTATGGCTGTGTTCTGAGGTTCATGTAGGTATATAAGCACTTCGTA
CTTTGCCACTTGTTTCATTTAGGCAACATTGTGCACATATCTAAGTAGTT
GGTCTTTGTAAAATTAGTTTGTTTGTCTTCAAAGTATATTGAGCAGTTTC
ATGACTCATTATTCAAAGGTTTGTCTAAATTAGAGAGAACTTTCATTTTG
CCTGGATTTAATCAGCATTTAGAATGTTTATAGCGATATCATTTTTAGTT
GAAAAAATCTCAACAAATTGACGCTTAGACAACTTAATAACACATTGCGG
ACGTTTTTAATGTACTGAATTAACGCCGAATTGAATTCGAGCTCGGTACC
CGGGGATCCTCTAGAGTCGACCTGCAGGCATGCAAGCTTAGCTTGAGCTT
GGATCAGATTGTCGTTTC
ad58-6620-17-1 Right border flanking
(SEQ ID NO: 446)
                    GTATAAGCCATGTTCCTTTCAGAGGGGACTGTACTTCTGT
AGATTACTTTCCCTCATTAACCAGATCTGGCCGGCCTACCCAGCTTTCTT
GTACAAAGTGGTGATAAACTATCAGTGTTAGATCCCCGACCGACCGCCCA
TCCTGGACGGCCTCGTGCATGCTGATGTTGTCAAAATCTTAGGATCCAAT
GAGTTTATCAGGTATACTTCTATCATGTATTGCTTGAGATTTTGGAGTGT
TAGTAAAGATTTCAATAAAAGAATTTTTTCAAACAAAATTTTGGGGGCTT
GAAGCTAATGTTTGGAAATATGTAACGGAGTTTAAATCTTTTGGCAGGCC
TATATATGCGGGAAACGCCTTGTGTAGAGTTCGCTATACTGGTGCTGGTC
CTTGTGTGTTGACTATTAGAACTACATCTTTTCCTGTTACCCCAATAACT
GAGTCAAAGAAAGCTACTATCTCTCAGATTGATCTCTCGAAATTCAAAGA
AGGTTTGTTTAGTATTATTCTCTTGTGCATAGCCTTTTTGSTTTTTTTTT
TTTTATAAAAAAAGTTGAGTATGCTTATTGCCCATTGC
```

Mapping of ETIPS

For each transgenic event containing a single copy insertion of the ETIP, the flanking sequence was taken following manual assembly and used as the query in a local BLAST analysis. There were a total of eight plants that had single copy integrations identified by this process (Table 16 and Table 17). A collection of 595,478 genomic derived shotgun sequences from Brassica oleracea were downloaded from the NCBI GSS database and formatted as a nucleotide BLAST database. The flanking ETIP sequences were then BLASTn compared to the database and all matches were manually examined. The most significant sequence match to the flanking ETIP sequence from the B. oleracea database was then taken and aligned against the online Brassica rapa genome sequence (http://brassicadb.org/brad/blastPage.php) where the position in the genome that had the most significant sequence match was also retrieved. In instances where a only the 5′ or 3′ flanking sequences provided significant matches with the B. oleracea genome sequences, it was assumed that the unaligned or unmatched sequence had either; identified missing sequence from the database, or that there had been significant genome rearrangements generated during the integration of the ETIP. For the samples that generated significant BLASTn matches from the analysis the flanking ETIP sequence, the most significant B. oleracea GSS matching sequence along with the most significant matching sequence from the B. rapa genome, were then manually aligned in Sequencher™ v5.0 software (Gene Codes Corp., Ann Arbor, Mich.) for each of the eight single copy ETIP plants. The three sequences were then compared and the most similar sequence from either of the diploid Brassica species compared to the flanking ETIP was designated the genome that the ETIP was located in. For the majority of the samples significant variation did exist between the two diploid Brassica genome sequences and the B. napus derived flanking ETIP sequence showed a predominant association with one or other of the diploid sequences. There were instances however, where there was insufficient sequence variation between the diploids and a linkage group assignment may have been possible but a sub-genome assignment was not possible. The specific genome location was then predicted from the location from the Brassica rapa genome sequence. In instances where the ETIP was identified as being integrated into the B. oleracea C genome, the comparative synteny between the diploid Brassica genomes described in Parkin et al. (Genetics 2005, 171: 765-781) was used to extrapolate the genomic location into the Brassica napus C sub-genome. In addition the sequences identified were BLASTn compared to the Arabidopsis thaliana genomes coding sequences (TAIR 9 CDS downloaded from http://arabidopsis.org/index.jsp) and the identity of any gene sequences disrupted were identified, as well as a confirmation of genomic location following the Arabidopsis Brassica synteny described in Schranz et al. (Trend in Plant Science 2006, 11, 11: 535-542).

TABLE 16
BLAST search and predicted the location of these above sequences
(predicted locations in Brassica napus genome).

| | One copy of each | LB | RB | |
|---|---|---|---|---|
| Event/Vector | cassette detected | Flanking | Flanking | Predicted |
| Name | by Southern | Sequence | Sequence | location |
| pDAS000036 | | | | |
| em02-5788-1-1 | yes | yes | yes | A6 |
| 228688 | | | | |
| ad58-5784-2-1 | yes | yes | yes | A8 |
| 232502 | | | | |
| ad58-5898-10-1 | yes | yes | yes | C7 |
| 237143 | | | | |
| pDAS000037 | | | | |
| lf31-6139-2-3 | yes | yes | yes | A5 |
| 234576 | | | | |
| bm56-6315-1-1 | yes | yes | yes | Genomic |
| 234703 | | | | Repeat |
| ad58-6372-1-1 | yes | yes | yes | A/C8 |
| 240653 | | | | |
| ad58-6620-4-1 | yes | yes | yes | C1 |
| 242268 | | | | |
| ad58-6620-17-1 | yes | yes | yes | A/C 3 |
| 242293 | | | | or 8 |

TABLE 17
description of single copy ETIP containing plant from the two constructs
pDAS000036 and 37, BLASTn result to a Brassica oleracea genome sequence
data base, potential disruption of gene sequence identified through Arabidopsis thaliana
gene comparison and predicted genome location.

| | BLASTn match to the | Predicted gene | |
|---|---|---|---|
| | C genome | disrupted | Predicted Location |
| pDAS000036 | | | |
| em02-5788-1-1 | Left border only value | At3g30775: proline | A6 |
| 228688 | e−175 | oxidase | |
| ad58-5784-2-1 | Left border only value | At1g50940: Electron | A8 |
| 232502 | e−134 | transfer fiavoprotein | |
| | | alpha | |
| ad58-5898-10-1 | Left border generated | No significant match to | C7 |
| 237143 | two significant matches | Arabidopsis gene | |
| | at value 0 and e−107 | | |
| pDAS000037 | | | |
| lf31-6139-2-3 | Right border only value | At3g08530: Clathrin | A5 |
| 234576 | e−105 | | |
| bm56-6315-1-1 | Both borders had large | N/A | Genomic Repeat |
| 234703 | numbers of matches e | | |
| | value 0 | | |
| ad58-6372-1-1 | Left and right border | No significant match to | Equivocal location: |
| 240653 | value e−103 and −80 | Arabidopsis genes | subgenome A or C on |
| | | | linkage group 8 |
| ad58-6620-4-1 | Left and right border | At4g27860: Vacuolar | C1 |
| 242268 | value e−154 and e−48 | ion transporter | |
| ad58-6620-17-1 | Left and right border | Borders identified | Equivocal location: |
| 242293 | value e−167 and e−94 | At5g20340 and | potentially sub-genome |
| | | At1g50930 | A or C and linkage |
| | | | group 3 or 8 |

The homozygous events are used to produce protoplasts via the previously described method. The protoplasts are subsequently co-transformed with a Zinc Finger Nuclease that is designed to target a Zinc Finger binding site which is incorporated within the ETIP sequence and a donor plasmid which shares homology with specific regions of the ETIP. The Zinc Finger Nuclease cleaves the ETIP locus and the donor plasmid is integrated within the genome of Brassica napus cells via homology directed repair. As a result of the integration of the donor plasmid, the partial DS-red transgene is repaired to a full length DS-red transgene. The expression of the now fully operational DS-red transgene is used to sort protoplast cells with a FACS method. Putative transgenic plants are sorted using the FACS method described in Example 7 and the isolated protoplasts are regenerated into mature plants. The integration of the donor plasmid is confirmed within the ETIP-targeted plants using molecular confirmation methods. As such, the ETIP locus serves as a site-specific locus for gene targeted integration of a donor polynucleotide sequence.

The genomic targeting locations provide genomic locations that do not alter the plants normal phenotype. The resulting events, wherein a transgene is targeted within an ETIP present no agronomically meaningful unintended differences when the ETIP events are compared to the control plants. In addition, the protein expression levels of transgenes integrated within the ETIP locus are robustly expressed and consistent and stable across multiple genomic locations. The disclosed genomic sequences of SEQ ID NO:431 to SEQ ID NO:446 provide genomic locations within the brassica genome that are targetable for the integration of gene expression cassettes comprising a transgene.

Molecular Confirmation of Fad2a Integration of ETIPS in Canola

Genomic DNA was extracted from leaf tissue of all putative transgenic plants using a DNeasy Plant Mini Kit™ (Qiagen) following the manufacturer's instructions, with the exception that tissue was eluted in 80 μl of AE buffer. Thirty milligrams of young leaf tissue from regenerated plants was snap frozen in liquid nitrogen before being ground to a powder.

Molecular characterization of the FAD2A locus was performed using three independent assays. Assays were designed and optimized using the following controls; characterized transgenic events comprising a single randomly integrated transgene, characterized transgenic event with five randomly integrated transgenes, wild-type canola c.v. DH12075 plants and non-template control reactions. The results from the three following molecular analyses are considered together in order to provide evidence for integration of the ETIP at FAD2A via HDR.

Identifying Transgene Integration by Real-Time Polymerase Chain Reaction

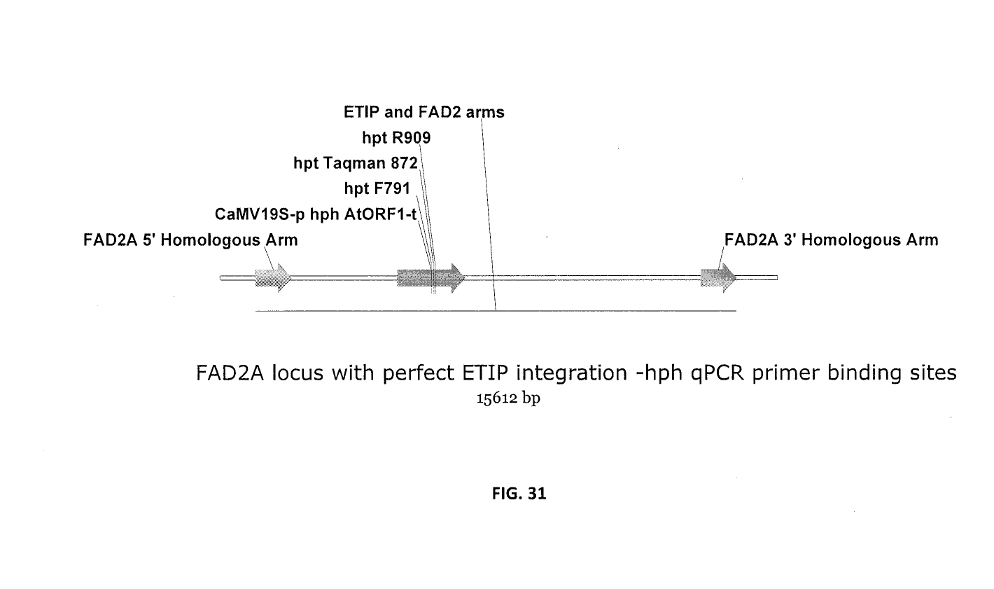

Four replicates of each plant were analyzed using primers specific to the hph (also described as hpt) target gene (SEQ ID NO:447, hpt F791 5′ CTTACATGCTTAGGATCGGACTTG 3′; SEQ ID NO:448, hpt R909 5′ AGTTCCAGCACCAGATCTAACG 3′; SEQ ID NO:449, hpt Taqman 872 5′ CCCTGAGCCCAAGCAGCATCATCG 3′ FAM) (FIG. 31) and reference gene encoding High Mobility Group protein I/Y (HMG I/Y) (SEQ ID NO:450, F 5′ CGGAGAGGGCGTGGAAGG 3′; SEQ ID NO:451, R 5′ TTCGATTTGCTACAGCGTCAAC 3′; SEQ ID NO:452, Probe 5′ AGGCACCATCGCAGGCTTCGCT 3′ HEX). The reactions were amplified using the following conditions: 95° C. for 10 minutes followed by 40 cycles of 95° C. for 30 seconds, 60° C. for 1 minute, with amplification data being captured at the end of each annealing step. Copy number was calculated using the ΔCq method, where ΔCq=Cq(target gene)−Cq(reference gene). Livak, K. J. and T. D. Schmittgen, Analysis of relative gene expression data using real-time quantitative PCR and the 2(-Delta Delta C(T)) Method. Methods, 2001. 25(4): p. 402-8. Plants with amplification of hph and HMG I/Y and a copy number of 0.5 or more were considered transgenic, while plants with a copy number of ≧0.5 and ≦1.2 were scored as putatively single copy. Amplification was performed on a BioRad CFX96 Touch™ Real-Time PCR Detection System with FastStart Universal Probe Master (ROX), (Roche, Basel, Switzerland).

Detection of Disrupted FAD2A ZFN Site

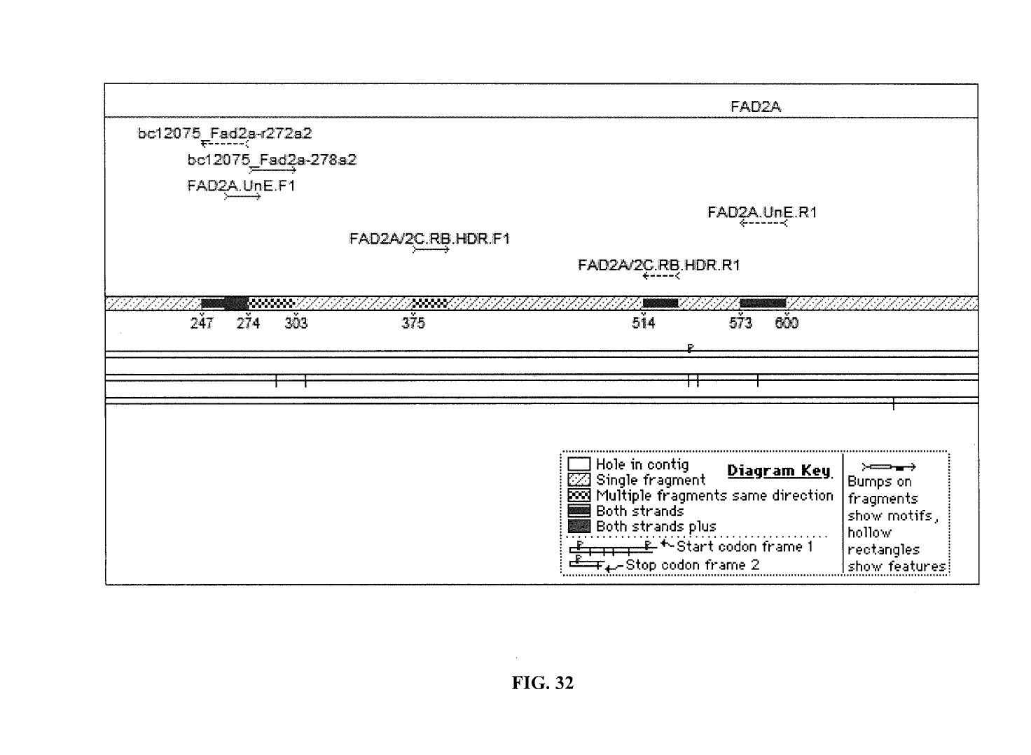

Each plant was analyzed for presence or absence of amplification of endogenous target in the disrupted locus test, which is a dominant assay. The assay is a SYBR® Green I qPCR assay and in singleplex, but with each reaction run simultaneously on the same PCR plate, targets an endogenous locus (FAD2A/2C.RB.UnE.F1, SEQ ID NO:453, 5′ CTTCCACTCCTTCCTCCTCGT*C 3′ and FAD2A/2C.RB.UnE.R1, 5′ SEQ ID NO:454, GCGTCCCAAAGGGTTGTTGA*G 3′) and the ZFN locus (locus at which the ZFN pDAB104010 binds and cuts the genome) (FAD2A.UnE.F1, SEQ ID NO:455, 5′ TCTCTACTGGGCCTGCCAGGG*C 3′ and FAD2A.UnE.R1, SEQ ID NO:456, 5′ CCCCGAGACGTTGAAGGCTAAGTACAA*A 3′) (FIG. 32). Both primer pairs were amplified using the following conditions: 98° C. for 30 seconds followed by 35 cycles of (98° C. for 10 seconds, 65° C. for 20 seconds, 72° C. for 90 seconds) then followed by 95° C. for 10 seconds then a melt analysis from 50° C. to 95° C. with 0.5° C. increments for 0.05 seconds and a plate read at each increment. The reaction conditions are listed in Table 18.

TABLE 18
Single reaction reagent components and
concentrations for PCR amplification.

| Reaction Components | Volume (μl) |
|---|---|
| 10 mM dNTP | 0.40 |
| 5X Phusion HF Buffer | 4.00 |
| Phusion Hot Start II High-Fidelity DNA Polymerase | 0.25 |
| (2 U/μl) (Thermo Scientific) | |
| Forward Primer 10 μM | 0.40 |
| Reverse Primer 10 μM | 0.40 |
| 1:10000 dilution of SYBR Green I dye (Invitrogen) | 1.00 |
| Molecular Biology Grade H₂O | 11.55 |
| Genomic DNA template (~20 ng/μl) | 2.00 |
| Total Volume | 20.00 |

Plants that had amplification of the endogenous target but no amplification of the ZFN target, were scored as positive for the disrupted locus test and were considered to have a disrupted ZFN locus. This assay was considered to be positive when the ZFN binding site on both alleles at the FAD2A locus have been disrupted.

PCR Detection of Transgene Integration at FAD2A Via Homology Directed Repair

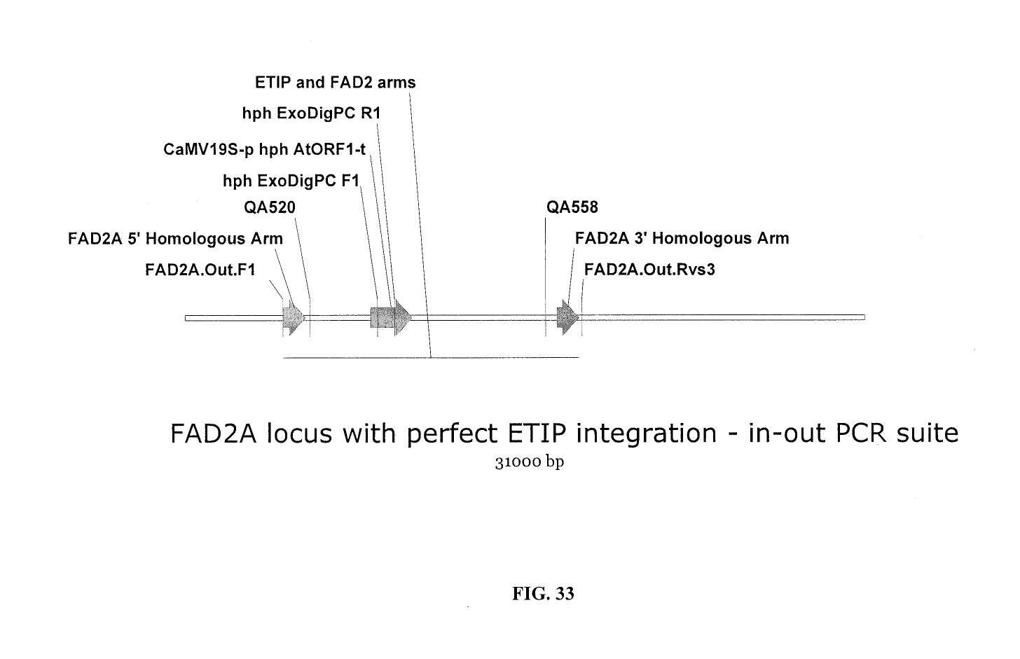

Each putative plant transformant was analysed using endpoint with PCR primers designed to amplify the transgene target hph (hph_ExoDigPC_F1, SEQ ID NO:457, 5′ TTGCGCTGACGGATTCTACAAGGA 3′ and hph_ExoDigPC_R1, SEQ ID NO:458, 5′ TCCATCAGTCCAAACAGCAGCAGA 3′), the FAD2A endogenous locus (FAD2A.Out.F1, SEQ ID NO:459, 5′ CATAGCAGTCTCACGTCCTGGT*C 3′ and FAD2A.Out.Rvs3, SEQ ID NO:460, 5′ GGAAGCTAAGCCATTACACTGTTCA*G 3′), the region spanning the 5′ end of any transgene inserted into the FAD2A locus via HDR, upstream of the transgene into the FAD2 A locus (FAD2A.Out.F1, SEQ ID NO:461, 5′ CATAGCAGTCTCACGTCCTGGT*C 3′ and QA520, SEQ ID NO:462, 5′ CCTGATCCGTTGACCTGCAG 3′) and the region spanning the 3′ end of any transgene inserted into the FAD2A locus via HDR, downstream of the transgene into the FAD2 A locus (QA558, SEQ ID NO:463, 5′ GTGTGAGGTGGCTAGGCATC 3′ and FAD2A.Out.Rvs3, SEQ ID NO:464, 5′ GGAAGCTAAGCCATTACACTGTTCA*G 3′) (FIG. 33). All primer pairs were amplified using the following conditions 98° C. for 30 seconds followed by 35 cycles of (98° C. for 10 seconds, 65° C. for 20 seconds, 72° C. for 90 seconds). Reaction reagent conditions are as described in Table 19.

TABLE 19
Single reaction reagent components and
concentrations for PCR amplification.

| Reaction Components | Volume (μl) |
|---|---|
| 5x Phusion HF Buffer | 6.00 |
| 10 mM dNTPs | 0.60 |
| Forward Primer 10 μM | 0.60 |
| Reverse Primer 10 μM | 0.60 |
| Phusion Hot Start II High-Fidelity DNA Polymerase | 0.25 |
| (2 U/μl) (Thermo Scientific) | |
| Molecular Biology Grade H₂O | 19.95 |
| Genomic DNA template (~20 ng/μl) | 2.00 |
| Total Volume | 30.0 |

Amplification of the 5′ transgene-genome flanking target and/or amplification of the 3′ transgene-genome flanking target indicated a putative insertion event. It must be noted that due to the approximately 1,000 bp FAD2A homology arms in the pDAS000130 cassette (comprising polynucleotide sequences with 100% sequence identity to the FAD2A regions immediately upstream and downstream of the ZFN cut site), the PCR reactions were subject to false positive PCR product amplification due to PCR chimerism arising from amplification of off-target ETIP integration events. Amplification of the hph target confirmed transgene integration had occurred Amplification of the FAD2A target suggests that the FAD2A locus is intact or contains only a partial insertion. Due to the size of the ETIP (11,462 bp for the ETIP cassettes or 13,472 bp including the FAD2A homologous arms and the ETIP cassettes) it is expected that the FAD2A primers would not amplify a product when an intact ETIP is integrated into the FAD2A locus.

Southern Detection of FAD2A Editing

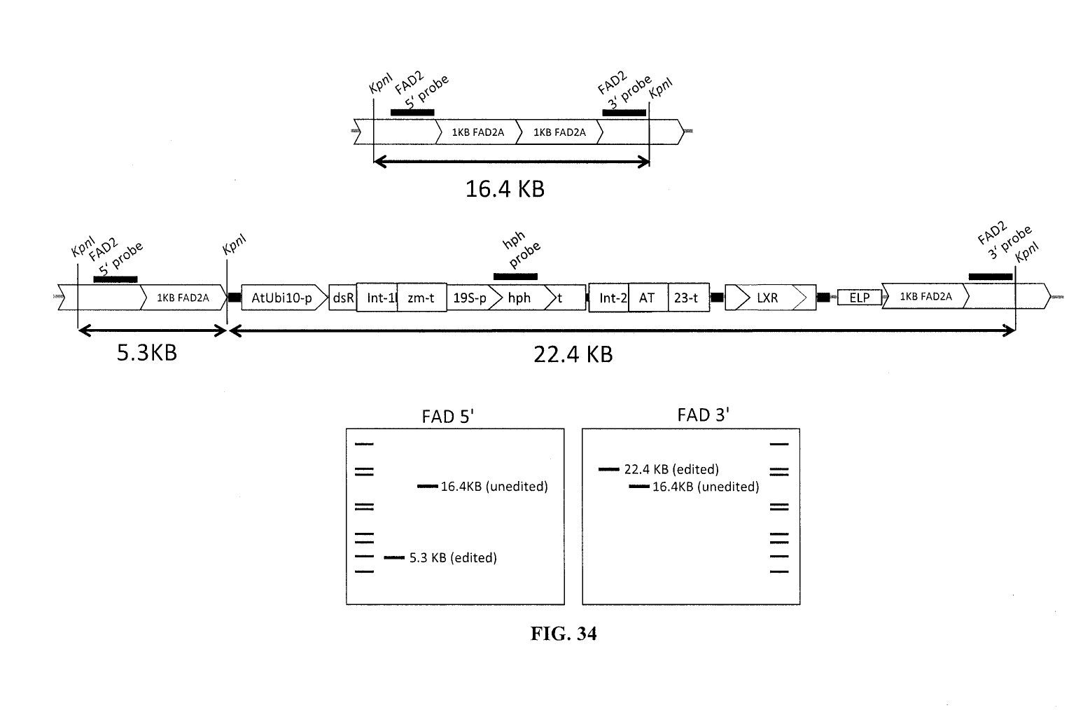

Plants that had amplification of either a 5′ genome-transgene flanking target product and/or amplification of a 3′ transgene-genome flanking target, or no amplification of the ZFN locus target, or both, were subject to Southern analysis for detection of transgene integration at the FAD2A locus. Genomic DNA was purified from 5 g of leaf tissue using a modified CTAB method (Maguire, T. L., G. G. Collins, and M. Sedgley A modified CTAB DNA extraction procedure for plants belonging to the family proteaceae. Plant Molecular Biology Reporter, 1994. 12(2): p. 106-109). Next, 12 μg of genomic DNA was digested with Kpn1-HF (New England BioLabs) and digestion fragments were separated by electrophoresis on a 0.8% agarose gel before transfer to membrane using a standard Southern blotting protocol. Primers to FAD2A 5′ target region (F, SEQ ID NO:465, 5′ AGAGAGGAGACAGAGAGAGAGT 3′ and R, SEQ ID NO:466, 5′ AGACAGCATCAAGATTTCACACA 3′), FAD2A 3′ target region (F, SEQ ID NO:467, 5′ CAACGGCGAGCGTAATCTTAG 3′ and R, SEQ ID NO:468, 5′ GTTCCCTGGAATTGCTGATAGG 3′) and hph (F, SEQ ID NO:469, 5′ TGTTGGTGGAAGAGGATACG 3′ and R, SEQ ID NO:470, 5′ ATCAGCAGCAGCGATAGC 3′) were used to generate probes to detect the presence of the ETIP within the FAD2A locus using the DIG EASY HYB SYSTEM® (Roche, South San Francisco, Calif.) following the manufacturer's instructions (FIG. 34). Hybridization was performed at 42° C. for FAD2A 5′ region, 45° C. for FAD2A 3′ region and 42° C. for detection of hph.

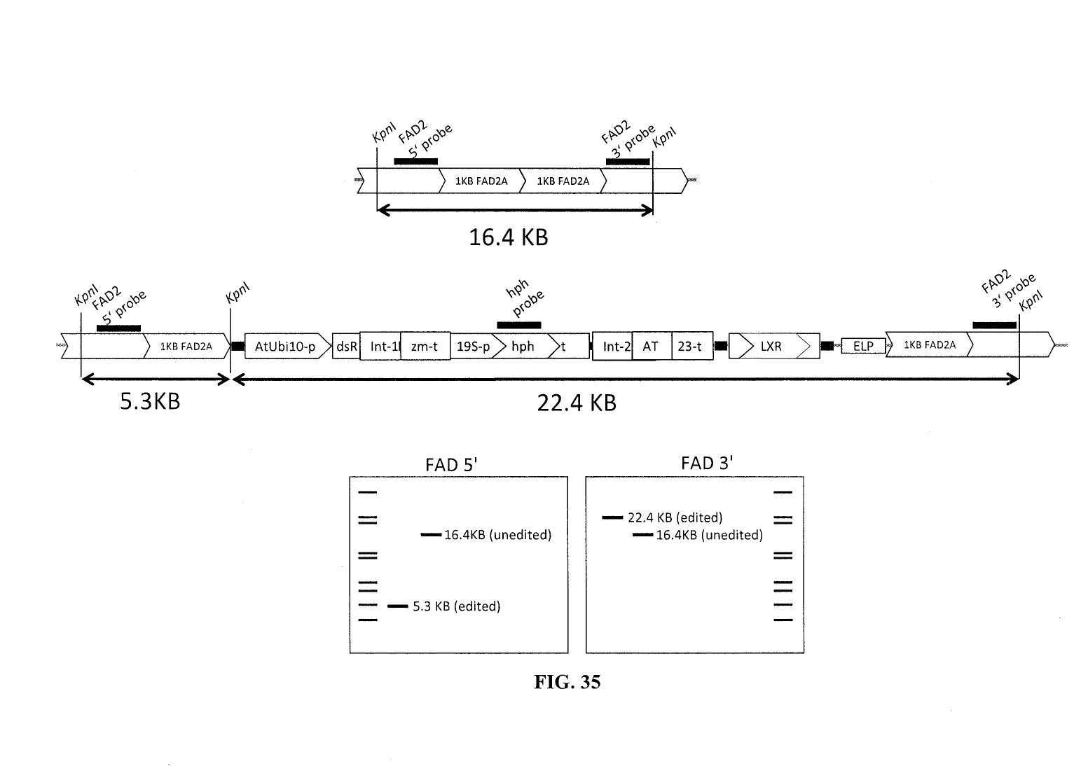

Membrane-bound genomic DNA was probed in a specific order; firstly FAD2A 5′ sequences were probed, then the FAD2A 3′ sequences were probe, and finally the hph sequences were probed (FIG. 35). The rational for this is as follows. The first probe (FAD2A 5′) is the diagnostic probe, and if the ETIP has integrated into FAD2A via perfect HDR, a 5,321 bp fragment will be visible on the membrane. The resulting band size is easily differentiated during electroporation and will sit close to the 5,148 bp fragments in the DIG labeled Roche DNA MOLECULAR WEIGHT MARKER III® (Roche, Indianapolis, Ind.). The second probe of the membrane is with the FAD2A 3′ probe and an edited plant will have a 22,433 bp fragment whereas an unedited plant will have a 16,468 bp fragment. The same 22,433 bp fragment identified with the FAD2A 3′ probe should also be bound by and identified with the hph probe. These fragments are difficult to differentiate on a gel as they are extremely large and it may be difficult to determine any difference between a fragment occurring above or below the largest, 21,226 bp fragment in the DIG labeled Roche DNA MOLECULAR WEIGHT MARKER III®. As such, these probes provide evidence that may strengthen the identification of ETIP integration into FAD2A via homology directed repair (HDR), by visualization of a 5 kb fragment using the FAD2A 5′ probe. The restriction enzyme, KpnI was the only suitable restriction endonuclease for use in this assay, as KpnI sites occurred in a single locus of the cut the ETIP cassette in a single locus, and was present in two sites of the FAD2A ZFN locus. One site was located upstream and the second site located downstream of the FAD2A homology arms. In addition, KpnI is not methylation sensitive, and is available as a recombinant enzyme with increased fidelity (New England Biolabs).

Results of Molecular and Southern Analysis

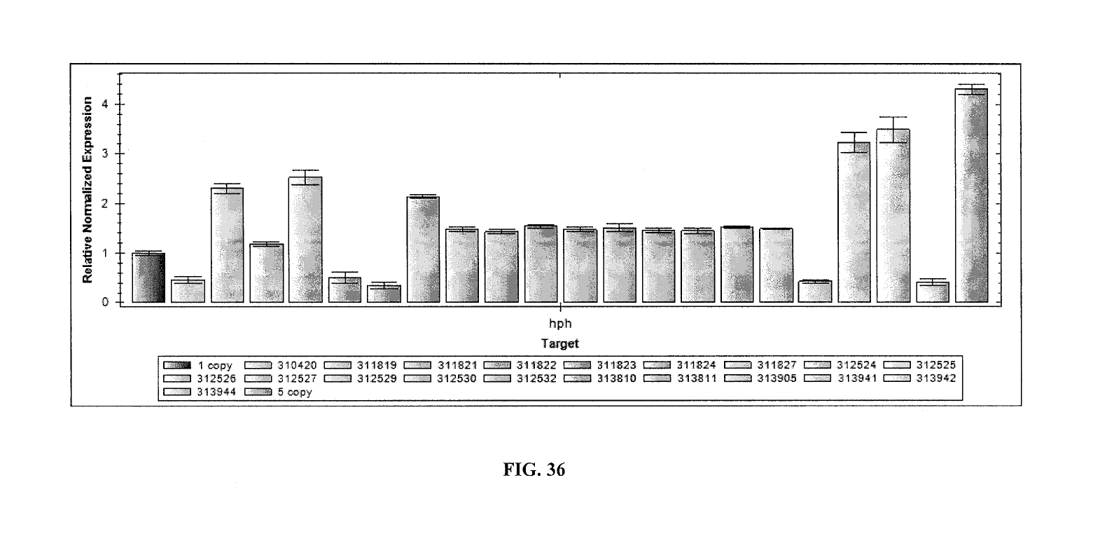

Following transfection, culturing, and selection the transgenic plants were transferred to soil. From this process, 139 plants survived and had tissue sampled for gDNA extraction and analysis. All 139 plants were analyzed for copy number estimation. Of these 139 plants, 56 were positive for the ETIP and 11 of the 56 positive plants had a putative single copy integration (FIG. 36) (Table 22). Of the 56 plants that were positive for ETIP integration, amplification of the FAD2A 5′-genome-transgene flanking sequence occurred in 7 plants. Amplification of the FAD2A 3′-transgene-genome flanking sequence did not occur in any of the 56 plants that were positive for ETIP integration. Additionally, of the 56 plants that were positive for transgene integration, 11 plants were positive for the disrupted locus qPCR test. Fourteen plants that were positive for amplification of the FAD2A 5′ genome-transgene flanking sequence and/or positive for the disrupted locus qPCR test were subject to Southern analysis, with the 3 probes described above. Of the 14 plants advanced for Southern analysis, all of the plants showed partial integration within the FAD2A locus, but none of these plants showed evidence of a complete full-length integration of the ETIP at the FAD2A locus via HDR when probed with the FAD2A 5′ probe, FAD2A 3′ and hph probes. No bands that appeared to be i) larger than WT and ii) identical to bands observed for those samples when probed with FAD2A 3′ probe (Table 20).

TABLE 20
Overview of outcomes from analysis of ETIP integration.

| | | Number | | | | | | |
|---|---|---|---|---|---|---|---|---|
| | | of plants | | | | | | |
| | | for which | | | | | | |
| | | qPCR | | Number of | | | | |
| | | copy | Number of | plants | Number | Number | Number | |
| Number | Number | number | plants | comprising | of ETIP/ | of ETIP/ | of locus | |
| of plants | of | analysis | positive | a putative | FAD2 | FAD2 in- | disrupted | ETIP |
| surviving | plants | was | for ETIP | single copy | in-out 5′ | out 3′ | qPCR | on-target |
| in soil | sampled | completed | integration | insert | reactions | reactions | tests | (Southern) |
| 139 | 139 | 139 | 56 | 11 | 7 | 0 | 9 | 0 |
| | | | | | (from 56) | (from 56) | (from 56) | (from 14) |

Results of Etip Trans Genic Canola Transformed with pDAS000130 and pDAB104010.

The transgenic Brassica napus events which are produced via transformation of pDAS000130 and pDAB104010 result in the integration of a single copy, full length T-strand insertion of the ETIP polynucleotide sequence from pDAS000130 within the FAD2A locus. Three to four events are fully characterized and confirmed to contain the integrated ETIP. The confirmation is completed using an in-out PCR amplification method, and further validated via Southern blot. The selected T₀ events are grown to the T₁ stage of development. The T₁ plants are re-screened to determine the zygosity of the integrated T-strand. Screened events are categorized as homozygous, hemizygous, or null.

The homozygous events are used to produce protoplasts via the previously described method. The protoplasts are subsequently co-transformed with a Zinc Finger Nuclease that is designed to target a Zinc Finger binding site which is incorporated within the ETIP sequence and a donor plasmid which shares homology with specific regions of the ETIP wherein the donor is integrated within the ETIP via an HDR mechanism. Likewise, the protoplasts are subsequently co-transformed with a Zinc Finger Nuclease that is designed to target a Zinc Finger binding site which is incorporated within the ETIP sequence and a donor plasmid which does not share homology with specific regions of the ETIP, wherein the donor is integrated within the ETIP via an non-homologous end joining mechanism. The Zinc Finger Nuclease cleaves the ETIP locus and the donor plasmid is integrated within the genome of Brassica napus cells via homology directed repair or non-homologous end joining. As a result of the integration of the donor plasmid, the partial DS-red transgene is repaired to a full length DS-red transgene. The expression of the now fully operational DS-red transgene is used to sort protoplast cells with a FACS method. Putative transgenic plants are sorted using the FACS method described in Example 7 and the isolated protoplasts are regenerated into mature plants. The integration of the donor plasmid is confirmed within the ETIP-targeted plants using molecular confirmation methods. As such, the ETIP locus serves as a site-specific locus for gene targeted integration of a donor polynucleotide sequence.

Results of Etip Transgenic Canola Transformed with Zinc Finger Nuclease and pDAS000271-pDAS000275 Etip Constructs

The transgenic Brassica napus events which are produced via transformation of ETIP and Zinc Finger Nuclease constructs result in the integration of a single copy, full length T-strand insertion of the ETIP polynucleotide sequence from pDAS000273 or pDAS275 within the FAD3A locus, and from pDAS000271, pDAS000272 or pDAS000274 into the FAD3C locus. Three to four events are fully characterized and confirmed to contain the integrated ETIP. The confirmation is completed using an in-out PCR amplification method, and further validated via Southern blot. The selected T₀ events are grown to the T₁ stage of development. The T₁ plants are res-screened to determine the zygosity of the integrated T-strand. Screened events are categorized as homozygous, hemizygous, or null.

The homozygous events are used to produce protoplasts via the previously described method. The protoplasts are subsequently co-transformed with a Zinc Finger Nuclease that is designed to target a Zinc Finger binding site which is incorporated within the ETIP sequence and a donor plasmid which shares homology with specific regions of the ETIP. The Zinc Finger Nuclease cleaves the ETIP locus and the donor plasmid is integrated within the genome of Brassica napus cells via homology directed repair. As a result of the integration of the donor plasmid, the partial DS-red transgene is repaired to a full length DS-red transgene. The expression of the now fully operational DS-red transgene is used to sort protoplast cells with a FACS method. Putative transgenic plants are sorted using the FACS method described in Example 7 and the isolated protoplasts are regenerated into mature plants. The integration of the donor plasmid is confirmed within the ETIP-targeted plants using molecular confirmation methods. As such, the ETIP locus serves as a site-specific locus for gene targeted integration of a donor polynucleotide sequence.

### Example 7

### FACS Based Sorting of Protoplast Cells

Brassica napus protoplasts that were transfected with the DS-Red control construct, pDAS000031, were sorted via FACS-mediated cell sorting using a BD Biosciences Influx-Cell sorter™ (San Jose, Calif.). The protoplast cells were isolated and transfected as described in Example 3. After the cells had been transfected with pDAS000031, the cells were sorted using the FACS sorter with the conditions described in Table 21.

TABLE 21
Conditions used for sorting protoplast cells transfected with pDAS000031.
Parameters

| | | |
|---|---|---|
| Drop frequency | 6.1 | KHz |
| Nozzle diameter | 200 | μm |
| Sheath pressure | 4 | psi |

| | |
|---|---|
| Recovery media | W5 media |
| Culture conditions | Bead type culture using sea-plaque |
| | agarose and sodium alginate |
| Sort criteria | Sorting based on chlorophyll autofluorescence, |
| | reporter gene expression (Ds-Red) |
| Sort recovery (%) | 50-75 |
| Viability post sorting (%) | >95 |

The protoplasts which expressed the DS-red transgene were sorted and isolated. The FACS isolated protoplasts were counted using the sorter. About 1×10⁵ to 1.8×10⁵ of cells were placed in a well of a 24-well micro titer plate on the first day after the FACS isolation. The cells were transferred to a bead culture for 5 to 20 days. Similar conditions were tested, wherein about 1×10⁴ of cells were placed in a well of a 2 or 4-well micro titer plate on the second day after the FACS isolation. The various conditions that were tested resulted in the recovery of cells at a viability or 95-98% of the total isolated protoplast cells. The FACS sorted protoplast cells were transferred to a bead culture for 3-20 days. The FACS sorted protoplast cells were regenerated into plants on media which contained 1.5 mg/mL of hygromycin using the above described protocol. The putative transgenic plants were confirmed to contain an intact T-strand insert from pDAS000031 via molecular conformation protocols.

Targeting of Etip Lines with ZFN Mediated Homologous Recombination Of Ds-Red

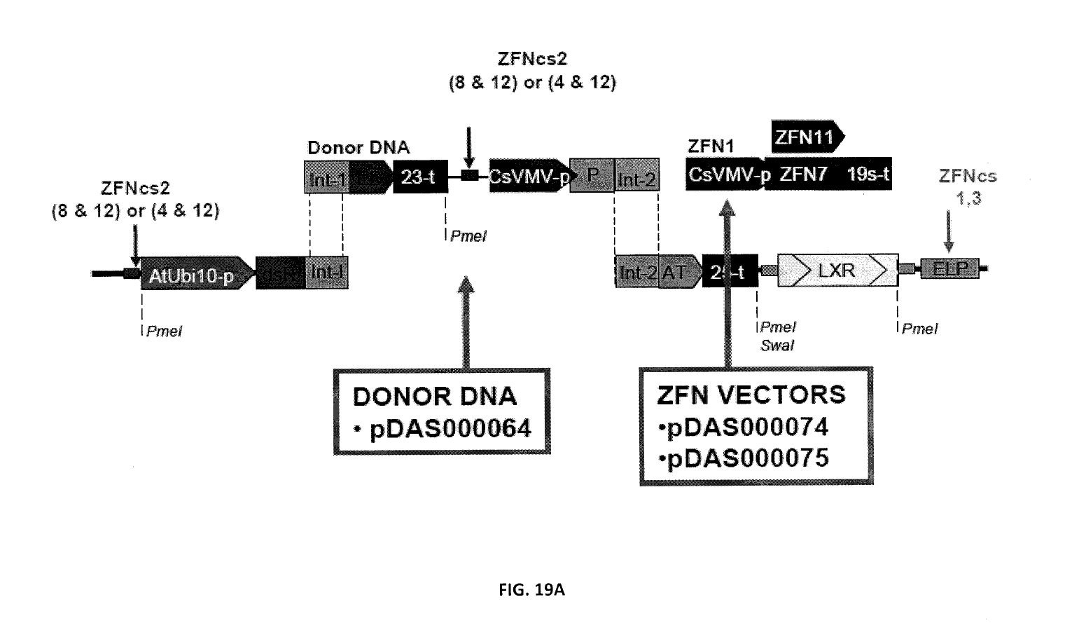

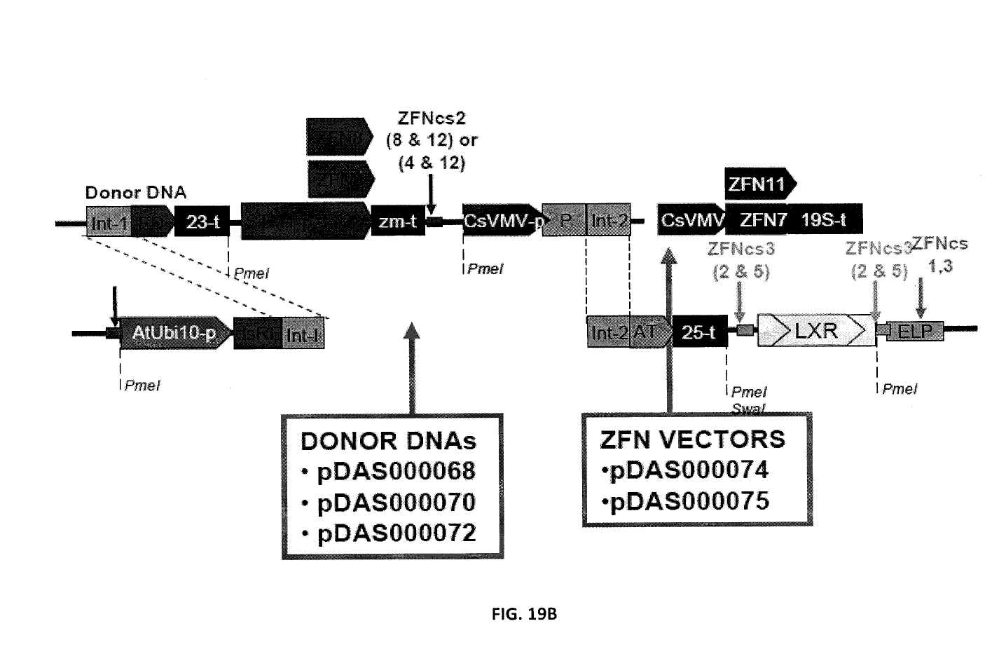

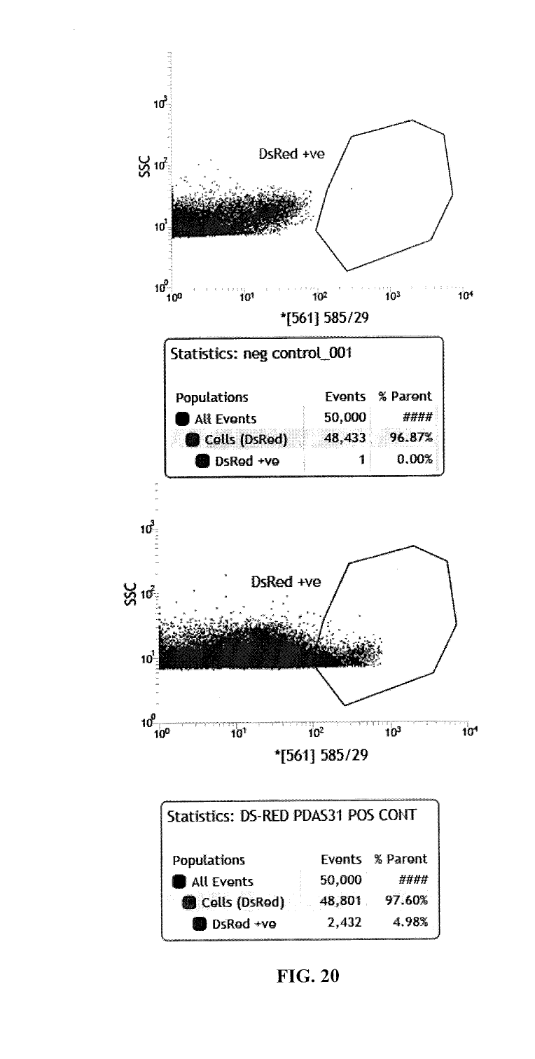

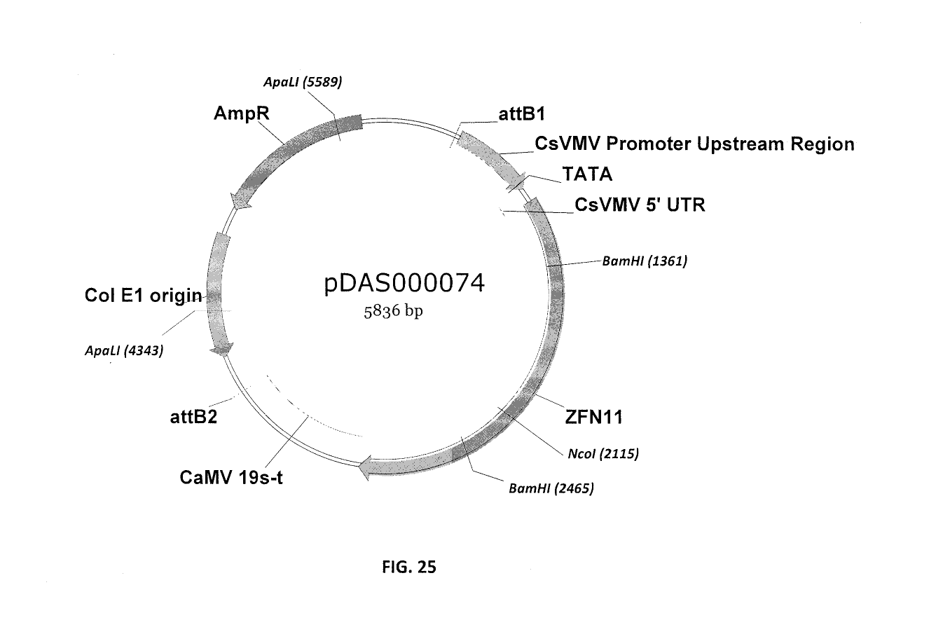

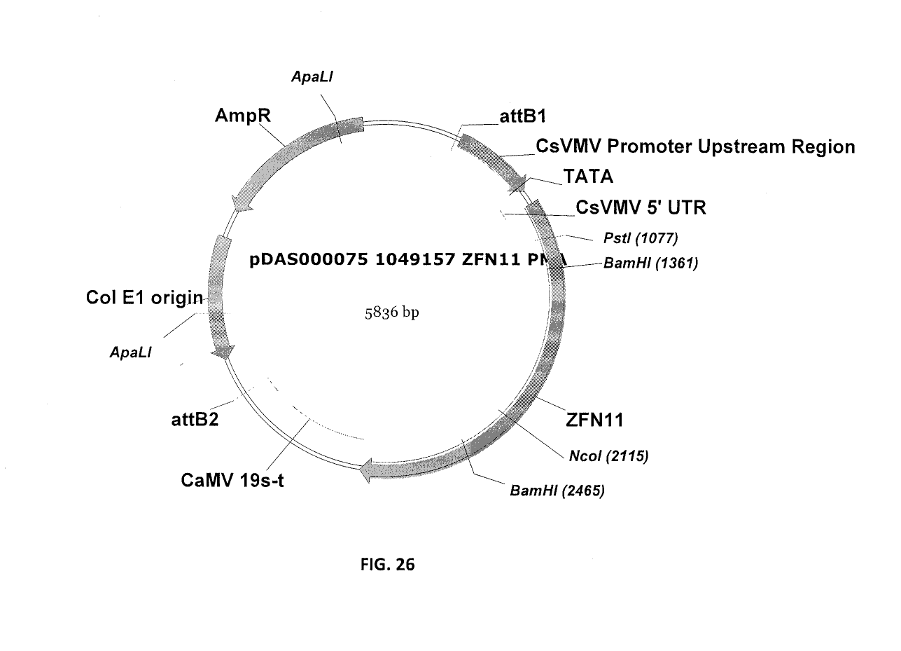

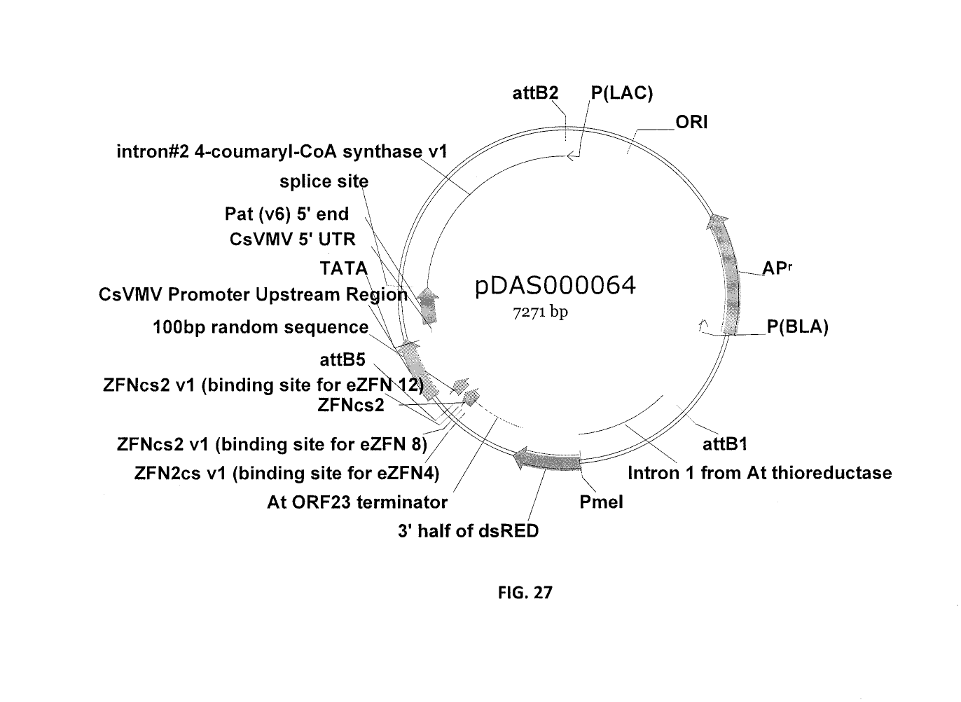

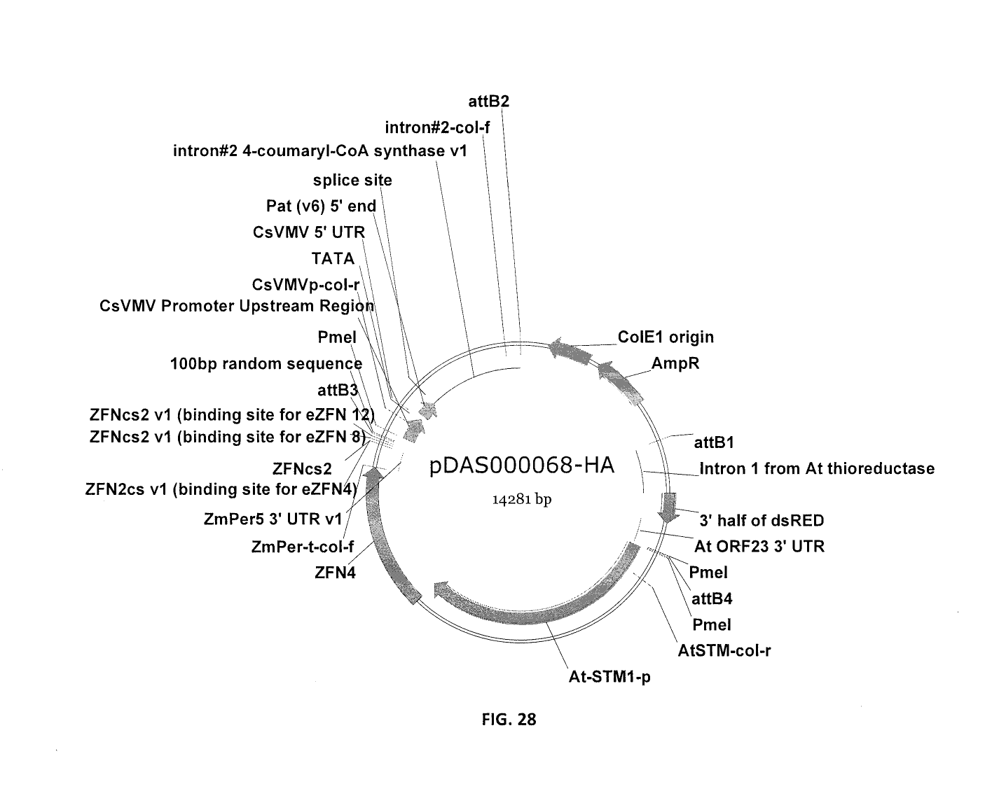

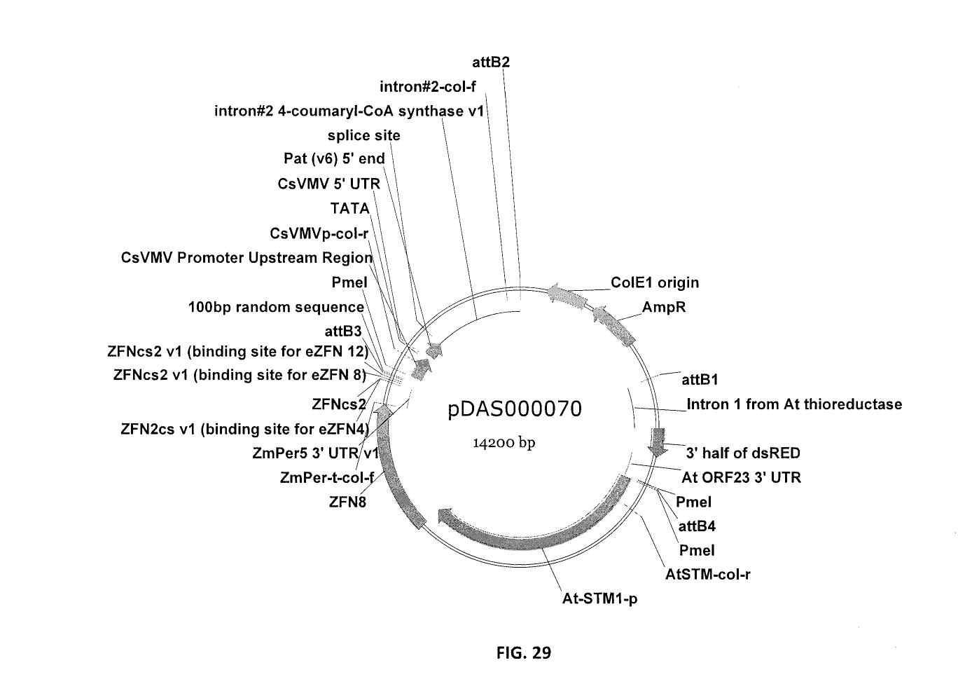

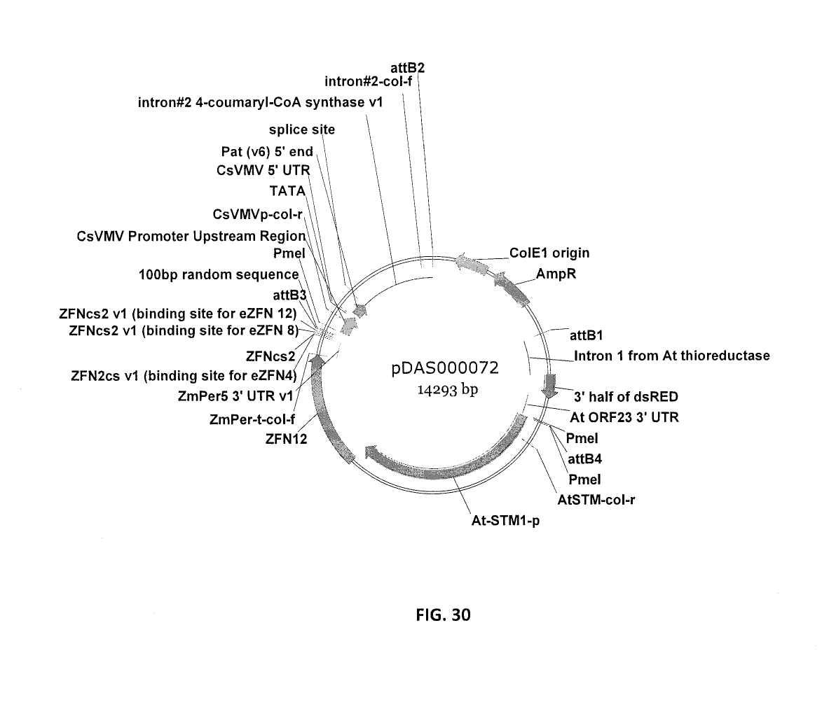

A canola line containing the T-strand insert from pDAS000036 was obtained and confirmed via molecular characterization to contain a full length, single copy of the T-strand. This canola event was labeled as pDAS000036-88 and was used to produce protoplasts via the previously described method. The protoplasts were isolated and ˜50,000 canola protoplast cells were subsequently co-transformed with a Zinc Finger Nuclease, either pDAS000074 (FIG. 25) or pDAS000075 (FIG. 26), that was designed to target the Zinc Finger binding sites incorporated within the ETIP sequence and a donor plasmid, pDAS000064, pDAS000068, pDAS000070, or pDAS000072 (FIG. 27, FIG. 28, FIG. 29, and FIG. 30, respectively), which shares homology with specific regions of the ETIP. FIG. 19 and FIG. 20 provide illustrations of the homology directed repair which results in the site-specific integration of the Ds-red transgene via Zinc Finger Nuclease mediated homologous recombination. The Zinc Finger Nuclease was designed to cleave the ETIP locus at a defined Zinc Finger binding sequence, thereby creating a double strand break within the genome. Next, the donor plasmid was integrated within the genome of Brassica napus protoplast cells via homology directed repair. The intron-1 and intron-2 regions of the donor plasmid share homology with the corresponding intron-1 and intron-2 regions of the ETIP locus. As a result of the integration of the donor plasmid, the partial DS-red transgene was repaired to a full length, highly expressing DS-red transgene. The expression of the fully operational DS-red transgene was used to sort protoplast cells with the above described FACS method. As such, the ETIP locus serves as a site-specific locus for targeted integration of a donor polynucleotide sequence. Finally, the isolated protoplasts can be sorted and regenerated into mature plants. The integration of the donor plasmid can be confirmed within the ETIP-targeted plants using molecular confirmation methods.

The donor plasmid DNA and ZFN plasmid DNA were mixed at various concentrations and used to transfect the canola protoplast cells containing Event pDAS000036-88, and the transgenic protoplast cells were sorted using the FACS transfection that was previously described. Table 22 describes the various transfection experiments and the DNA concentrations which were used for the transfection of the canola protoplasts containing Event pDAS000036-88. The ZFN and donor plasmid DNA was isolated and prepared for the transfections via the previously described methods.

TABLE 22
Donor plasmids and Zinc Finger Nuclease constructs used for the
ETIP targeting experiments. The DNA concentrations were used at
the indicated ratio of donor to Zinc Finger Nuclease, for a total
concentration of 30 micrograms of plasmid DNA per transfection.

| | | DONOR | ZFN | |
|---|---|---|---|---|
| | | PLASMID | PLASMID | TOTAL |
| REACTIONS | PLASMIDS | DNA (μg) | DNA (μg) | (μg) |
| 1 | pDAS000074 | — | 30 | 30 |
| 2 | pDAS000075 | — | 30 | 30 |
| 3 | pDAS000064 + | 26 | 4 | 30 |
| | pDAS000074 | | | |
| 4 | pDAS000064 + | 26 | 4 | 30 |
| | pDAS000075 | | | |
| 5 | pDAS000068 + | 28 | 2 | 30 |
| | pDAS000074 | | | |
| 6 | pDAS000068 + | 28 | 2 | 30 |
| | pDAS000075 | | | |
| 7 | pDAS000070 + | 28 | 2 | 30 |
| | pDAS000074 | | | |
| 8 | pDAS000070 + | 28 | 2 | 30 |
| | pDAS000075 | | | |
| 9 | pDAS000072 + | 28 | 2 | 30 |
| | pDAS000074 | | | |
| 10 | pDAS000072 + | 28 | 2 | 30 |
| | pDAS000075 | | | |
| 11 | pDAS000064 | 30 | — | 30 |
| 12 | pDAS000068 | 30 | — | 30 |
| 13 | pDAS000070 | 30 | — | 30 |
| 14 | pDAS000072 | 30 | — | 30 |

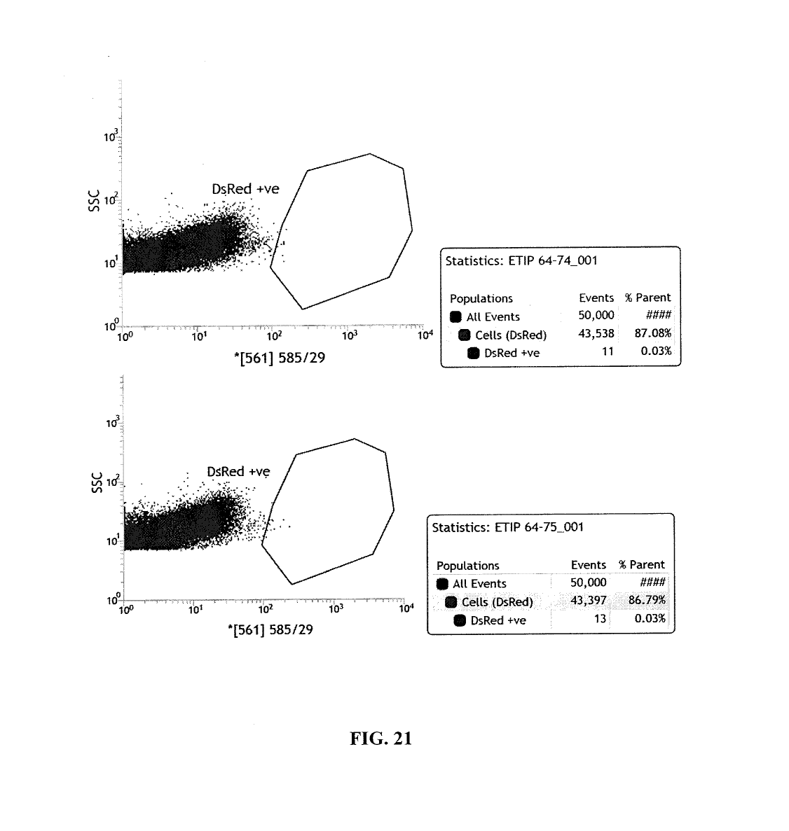

After the transfection experiments were completed the protoplasts were incubated at room temperature for 48 hours and sorted using the above described FACS protocol. Each experiment was sorted independently and Zinc Finger-mediated introgression of a transgene was confirmed via identification of individual events which expressed the DS-red transgene. FIGS. 21-24 show the results of the FACS sorting. As the results depicted in the graphs indicate, multiple events were produced which contained an intact fully integrated DS-red transgene. These multiple Ds-Red events were the result of Zinc Finger Nuclease mediated integration of the donor DNA construct within the ETIP genomic locus. This site-specific integration resulted in a highly expressing, complete copy of the Ds-Red transgene. The frequency of the Ds-Red transgene expression ranged from about 0.03-0.07% of the total canola protoplast cells (˜50,000). However, the frequency of transfection efficiency for the Zinc Finger Nuclease mediated integration of the donor DNA construct within the ETIP genomic locus was much higher and ranged from about 0.07-0.64%.

FIG. 21 shows the results of the transfections in which the donor plasmid and ZFN plasmid were co-transformed. The top graph, wherein donor, pDAS000064, and the Zinc Finger Nuclease, pDAS000074, were co-transformed at a ratio of 26 μs to 4 μg of plasmid DNA resulted in the Zinc Finger Nuclease mediated integration of the donor DNA construct within the ETIP genomic locus at a recombination frequency of about 0.03% of the ˜50,000 canola protoplast cells. In actuality, the recombination frequency is much higher. Of the ˜50,000 canola protoplast cells which were provided during the transfection experiment, only about 10-30% of these canola protoplast cells are actually transformed. As such, the actual Zinc Finger Nuclease mediated integration of the donor DNA construct within the ETIP genomic locus transfection efficiency ranges from about 0.22-0.07%. Similarly the bottom graph, wherein donor, pDAS000064, and the Zinc Finger Nuclease, pDAS000075, were co-transformed at a ratio of 26 μg to 4 μg of plasmid DNA resulted in the Zinc Finger Nuclease mediated integration of the donor DNA construct within the ETIP genomic locus at a recombination frequency of about 0.03% of the ˜50,000 canola protoplast cells. In actuality, the recombination frequency is much higher. Of the ˜50,000 canola protoplast cells which were provided during the transfection experiment, only about 10-30% of these cells are actually transfected. As such, the actual Zinc Finger Nuclease mediated integration of the donor DNA construct within the ETIP genomic locus transfection efficiency ranges from about 0.26-0.08%. The results of the zinc finger mediated homology directed repair are significantly greater than the negative control experiments, wherein only one protoplast of ˜50,000 was identified to have red fluorescence, thereby resulting in a recombination frequency of 0.00%, as shown in FIG. 20.

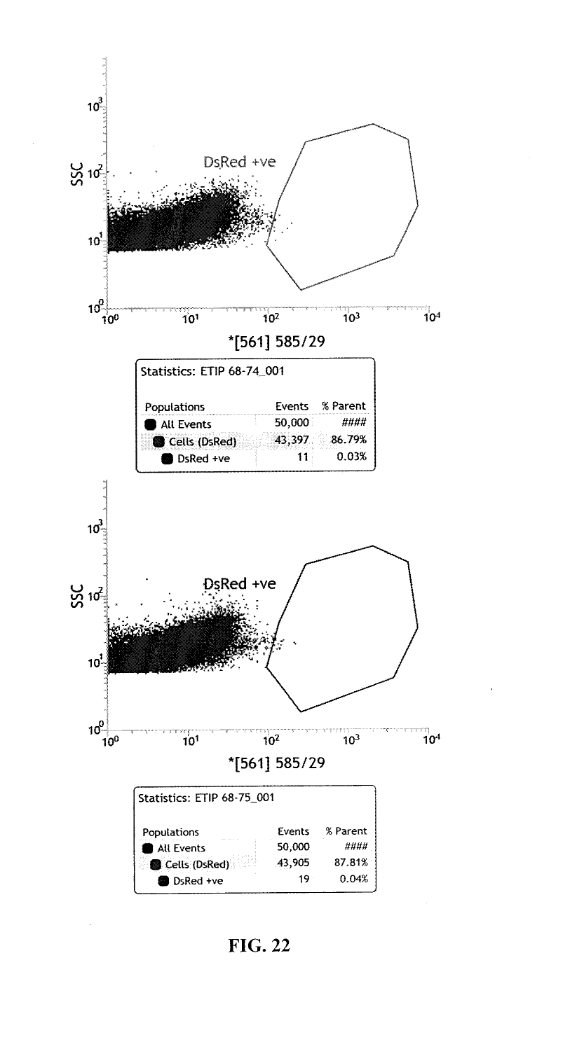

FIG. 22 shows the results of the transfections in which the donor plasmid and ZFN plasmid were co-transformed. The top graph, wherein donor, pDAS000068, and the Zinc Finger Nuclease, pDAS000074, were co-transformed at a ratio of 28 μg to 2 μg of plasmid DNA resulted in the Zinc Finger Nuclease mediated integration of the donor DNA construct within the ETIP genomic locus at a recombination frequency of about 0.03% of the ˜50,000 canola protoplast cells. In actuality, the recombination frequency is much higher. Of the ˜50,000 canola protoplast cells which were provided during the transfection experiment, only about 10-30% of these canola protoplast cells are actually transformed. As such, the actual Zinc Finger Nuclease mediated integration of the donor DNA construct within the ETIP genomic locus transfection efficiency ranges from about 0.22-0.07%. Similarly the bottom graph, wherein donor, pDAS000068, and the Zinc Finger Nuclease, pDAS000075, were co-transformed at a ratio of 28 μg to 2 μg of plasmid DNA resulted in the Zinc Finger Nuclease mediated integration of the donor DNA construct within the ETIP genomic locus at a recombination frequency of about 0.04% of the ˜50,000 canola protoplast cells. In actuality, the recombination frequency is much higher. Of the ˜50,000 canola protoplast cells which were provided during the transfection experiment, only about 10-30% of these cells are actually transfected. As such, the actual Zinc Finger Nuclease mediated integration of the donor DNA construct within the ETIP genomic locus transfection efficiency ranges from about 0.38-0.12%. The results of the zinc finger mediated homology directed repair are significantly greater than the negative control experiments, wherein only one protoplast of ˜50,000 was identified to have red fluorescence, thereby resulting in a recombination frequency of 0.00%, as shown in FIG. 20.

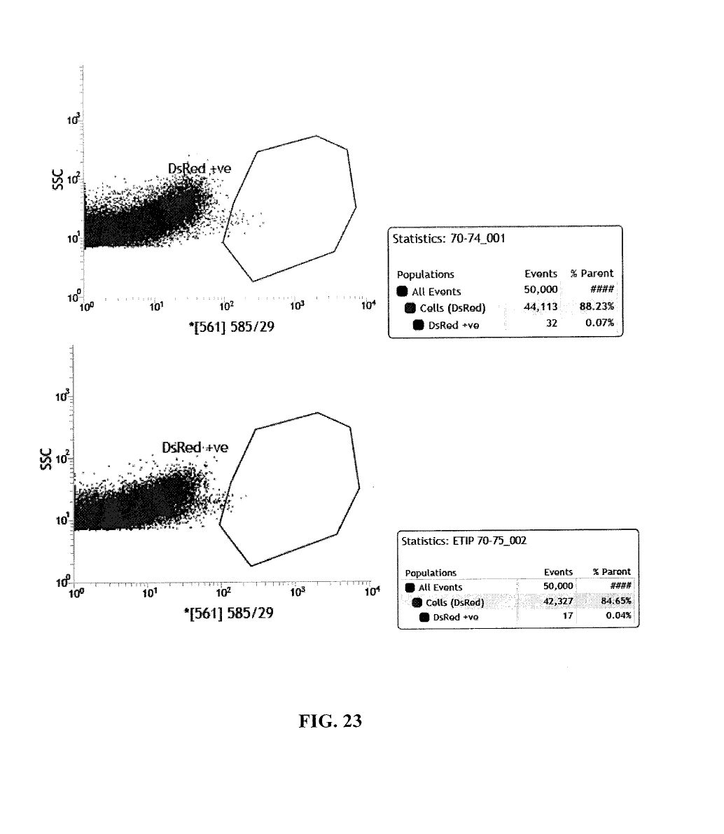

FIG. 23 shows the results of the transfections in which the donor plasmid and ZFN plasmid were co-transformed. The top graph, wherein donor, pDAS000070, and the Zinc Finger Nuclease, pDAS000074, were co-transformed at a ratio of 28 μg to 2 μg of plasmid DNA resulted in the Zinc Finger Nuclease mediated integration of the donor DNA construct within the ETIP genomic locus at a recombination frequency of about 0.07% of the ˜50,000 canola protoplast cells. In actuality, the recombination frequency is much higher. Of the ˜50,000 canola protoplast cells which were provided during the transfection experiment, only about 10-30% of these canola protoplast cells are actually transformed. As such, the actual Zinc Finger Nuclease mediated integration of the donor DNA construct within the ETIP genomic locus transfection efficiency ranges from about 0.64-0.21%. Similarly the bottom graph, wherein donor, pDAS000070, and the Zinc Finger Nuclease, pDAS000075, were co-transformed at a ratio of 28 μg to 2 μg of plasmid DNA resulted in the Zinc Finger Nuclease mediated integration of the donor DNA construct within the ETIP genomic locus at a recombination frequency of about 0.04% of the ˜50,000 canola protoplast cells. In actuality, the recombination frequency is much higher. Of the ˜50,000 canola protoplast cells which were provided during the transfection experiment, only about 10-30% of these cells are actually transfected. As such, the actual Zinc Finger Nuclease mediated integration of the donor DNA construct within the ETIP genomic locus transfection efficiency ranges from about 0.34-0.11%. The results of the zinc finger mediated homology directed repair are significantly greater than the negative control experiments, wherein only one protoplast of ˜50,000 was identified to have red fluorescence, thereby resulting in a recombination frequency of 0.00%, as shown in FIG. 20.

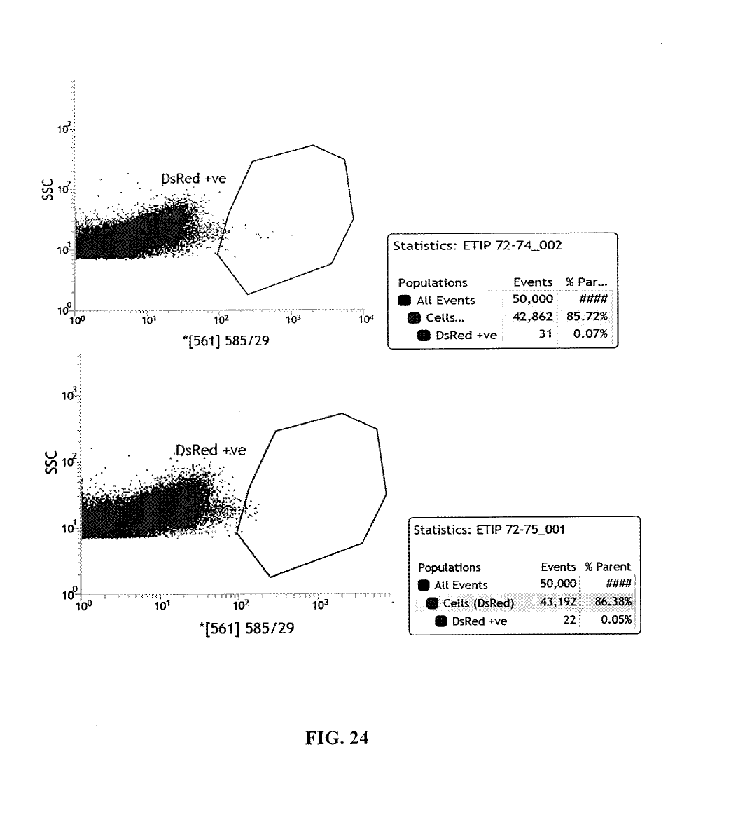

FIG. 24 shows the results of the transfections in which the donor plasmid and ZFN plasmid were co-transformed. The top graph, wherein donor, pDAS000072, and the Zinc Finger Nuclease, pDAS000074, were co-transformed at a ratio of 28 μg to 2 μg of plasmid DNA resulted in the Zinc Finger Nuclease mediated integration of the donor DNA construct within the ETIP genomic locus at a recombination frequency of about 0.07% of the ˜50,000 canola protoplast cells. In actuality, the recombination frequency is much higher. Of the ˜50,000 canola protoplast cells which were provided during the transfection experiment, only about 10-30% of these canola protoplast cells are actually transformed. As such, the actual Zinc Finger Nuclease mediated integration of the donor DNA construct within the ETIP genomic locus transfection efficiency ranges from about 0.62-0.20%. Similarly the bottom graph, wherein donor, pDAS000072, and the Zinc Finger Nuclease, pDAS000075, were co-transformed at a ratio of 28 μg to 2 μg of plasmid DNA resulted in the Zinc Finger Nuclease mediated integration of the donor DNA construct within the ETIP genomic locus at a recombination frequency of about 0.05% of the 18 50,000 canola protoplast cells. In actuality, the recombination frequency is much higher. Of the ˜50,000 canola protoplast cells which were provided during the transfection experiment, only about 10-30% of these cells are actually transfected. As such, the actual Zinc Finger Nuclease mediated integration of the donor DNA construct within the ETIP genomic locus transfection efficiency ranges from about 0.44-0.14%. The results of the zinc finger mediated homology directed repair are significantly greater than the negative control experiments, wherein only one protoplast of ˜50,000 was identified to have red fluorescence, thereby resulting in a recombination frequency of 0.00%, as shown in FIG. 20.

Selected explants were transferred and cultured upon regeneration media containing phosphothrinocin. After the culturing period the surviving explants were transferred to elongation medium and root induction medium for culturing and plant development. Whole plants that consisted of developed root and shoot structures were transferred into soil and further propagated in the greenhouse. The tissue culture process utilized media and culture conditions as previously described above. The results of plants produced from the tissue culturing process are shown in Table 23 below.

TABLE 23
Results of tissue culturing process.

| | No. of explants | No. of explants | No. of shoots | | |
|---|---|---|---|---|---|
| | transferred to | surviving in | surviving in shoot | No. of shoots | No. of rooted plants |
| | regeneration media: | regeneration media: | elongation media: | surviving in | transferred |
| Construct | B2-2 PPT | B2-2 PPT | SEM- 2 PPT | RIM - 2 PPT | to soil |
| pDAS000064 + | 4021 | 36 | 74 | 1 | — |
| pDAS000074-I | | | | | |
| pDAS000064 + | 1300 | 90 | 13 | 1 | 1 |
| pDAS000074-II | | | | | |
| pDAS000064 + | 1760 | 15 | 36 | 2 | — |
| pDAS000074-III | | | | | |
| pDAS000068 + | 1700 | 100 | 4 | 8 | 2 |
| pDAS000074-I | | | | | |
| pDAS000068 + | 1630 | — | 29 | 15 | — |
| pDAS000074-II | | | | | |
| pDAS000068 + | 2523 | 30 | 11 | 1 | — |
| pDAS000074-III | | | | | |
| pDAS000070 + | 2084 | 10 | 34 | 1 | — |
| pDAS000074-I | | | | | |
| pDAS000070 + | 4151 | — | 88 | 7 | — |
| pDAS000074-II | | | | | |
| pDAS000070 + | 1480 | 415 | 14 | 0 | — |
| pDAS000074-III | | | | | |
| pDAS000072 + | 1980 | 7 | 19 | 16 | — |
| pDAS000074-I | | | | | |
| pDAS000072 + | 1050 | 0 | 0 | 0 | — |
| pDAS000074-II | | | | | |
| pDAS000072 + | 1200 | — | 2 | 0 | — |
| pDAS000074-III | | | | | |
| pDAS000064 + | 556 | — | 8 | 1 | — |
| pDAS000074-I | | | | | |
| pDAS000064 + | 581 | 13 | 7 | — | — |
| pDAS000074-II | | | | | |
| pDAS000064 + | 1160 | 90 | 17 | 1 | — |
| pDAS000074-III | | | | | |
| pDAS000068 + | 516 | 0 | 13 | — | — |
| pDAS000074-I | | | | | |
| pDAS000068 + | 1725 | 55 | 19 | 3 | — |
| pDAS000074-II | | | | | |
| pDAS000068 + | 930 | 57 | 0 | — | — |
| pDAS000074-III | | | | | |
| pDAS000070 + | 600 | 8 | 3 | — | — |
| pDAS000074-I | | | | | |
| pDAS000070 + | 4410 | 1410 | 360 | 3 | — |
| pDAS000074-II | | | | | |
| pDAS000070 + | 2350 | 108 | 51 | 8 | — |
| pDAS000074-III | | | | | |
| pDAS000072 + | 1660 | 10 | 19 | 3 | 1 |
| pDAS000074-I | | | | | |
| pDAS000072 + | 175 | — | 13 | — | — |
| pDAS000074-II | | | | | |
| pDAS000072 + | 250 | 9 | 2 | — | — |
| pDAS000074-III | | | | | |

| | | No. of explants | No. of explants | No. of shoots | | |
|---|---|---|---|---|---|---|
| | No. of | transferred to | surviving in | recovered on shoot | No. of shoots | No. of rooted plants |
| | protoplasts | regeneration | regeneration | elongation media: | transferred to | transferred |
| CONSTRUCTS | recovered | media: B2-2 PPT | media: B2-2 PPT | SEM- 2 PPT | RIM - 2 PPT | to soil |
| pDAS000064 + | 3 × 10⁵ | — | — | — | — | — |
| pDAS000074 | | | | | | |
| pDAS000068 + | 1 × 10⁵ | 114 | 12 | 1 | — | — |
| pDAS000074 | | | | | | |
| pDAS000070 + | 3 × 10⁵ | 478 | 391 | — | — | — |
| pDAS000074 | | | | | | |
| pDAS000072 + | 3 × 10⁵ | 81 | 12 | — | — | — |
| pDAS000074 | | | | | | |
| pDAS000064 + | 3 × 10⁵ | 38 | 7 | — | — | — |
| pDAS000074 | | | | | | |
| pDAS000068 + | 3 × 10⁵ | — | — | — | — | — |
| pDAS000074 | | | | | | |
| pDAS000070 + | 1 × 10⁵ | 80 | 7 | 1 | — | — |
| pDAS000074 | | | | | | |
| pDAS000072 + | 3 × 10⁵ | 7 | 3 | — | — | — |
| pDAS000074 | | | | | | |

The FACS sorting method is directly applicable to screen any fluorescent transgene sequence and is used to isolate a proportion of any protoplast, herein Brassica napus protoplast cells that are targeted with a fluorescent transgene via homology mediated repair within a specific site in the ETIP region within a genomic locus.

While certain exemplary embodiments have been described herein, those of ordinary skill in the art will recognize and appreciate that many additions, deletions, and modifications to the exemplary embodiments may be made without departing from the scope of the following claims. In addition, features from one embodiment may be combined with features of another embodiment.

What may be claimed is:

1. A method for generating a plant from a population of plant cells comprising isolating a plant protoplast comprising a polynucleotide of interest, the method comprising:
providing a population of plant protoplasts having at least one protoplast comprising a polynucleotide of interest and a fluorescent marker, wherein the population is substantially free of plant protoplasts comprising the fluorescent marker and not comprising the polynucleotide of interest; wherein the plant protoplast is encapsulated by sodium alginate;
separating the at least one protoplast comprising the polynucleotide of interest and the fluorescent marker from the remaining plant protoplasts in the population, thereby isolating a plant protoplast comprising the polynucleotide of interest;
regenerating a plant from said isolated plant protoplast; and
culturing said plant.

2. The method according to claim 1, wherein separating the at least one protoplast comprises utilizing flow cytometry.

3. The method according to claim 1, wherein separating the at least one protoplast comprises utilizing fluorescence-activated cell sorting (FACS).

4. The method according to claim 1, wherein the fluorescent marker is a fluorescent polypeptide that is expressed from a polynucleotide in the plant protoplast.

5. The method according to claim 1, wherein the polynucleotide of interest encodes a polypeptide of interest.

6. The method according to claim 5, wherein the polypeptide of interest is a zinc-finger nuclease.

7. The method according to claim 1, wherein the population of plant protoplasts is obtained from a plant tissue.

8. The method according to claim 1, comprising separating a plurality of protoplasts comprising the polynucleotide of interest and the fluorescent marker.

9. The method according to claim 1, wherein the plant is a monocot or dicot.

10. A plant regenerated by isolating a plant protoplast comprising a polynucleotide of interest integrated into the genome of the plant protoplast, the method comprising:
providing a population of plant protoplasts having at least one protoplast comprising a polynucleotide of interest and a fluorescent marker; wherein the plant protoplast is encapsulated by sodium alginate;
recovering microcalli from the population of protoplasts comprising the polynucleotide of interest and the fluorescent marker wherein the at least one protoplast comprises the polynucleotide of interest and the fluorescent marker has been transformed with the polynucleotide of interest and a polynucleotide encoding the fluorescent marker;
regenerating a plant from said microcalli; and
culturing said plant.

11. The method according to claim 9, wherein the polynucleotide of interest and the polynucleotide encoding the fluorescent marker were both present in a nucleic acid molecule used to transform the at least one protoplast comprising the polynucleotide of interest and the fluorescent marker.

12. The method according to claim 9, wherein the polynucleotide of interest and the polynucleotide encoding the fluorescent marker are integrated in the genome of the at least one protoplast comprising the polynucleotide of interest and the fluorescent marker.

13. The method according to claim 12, wherein the polynucleotide of interest and the polynucleotide encoding the fluorescent marker are integrated in a site-specific manner in the genome of the at least one protoplast.

14. The method according to claim 13, wherein the polynucleotide of interest and the polynucleotide encoding the fluorescent marker are integrated in a site-specific manner by utilizing a zinc-finger nuclease.

15. A method for producing a transgenic plant, the method comprising:
providing a population of plant protoplasts having at least one protoplast comprising a polynucleotide of interest and a fluorescent marker, wherein the at least one protoplast comprises a site-specific nuclease, such that the polynucleotide of interest is capable of being integrated in the genome of the at least one plant protoplast by homologous recombination at a recognition site of the site-specific nuclease and wherein the plant protoplast is encapsulated by sodium alginate;
separating the at least one protoplast comprising the polynucleotide of interest and the fluorescent marker from the remaining plant protoplasts in the population;
regenerating the transgenic plant from the at least one protoplast; and
culturing said transgenic plant.

16. The plant produced by the method according to claim 15, wherein the plant produces a polypeptide of interest that is encoded by the polynucleotide of interest.

17. The plant produced by the method according to claim 15, wherein the plant comprises a value-added trait conferred to the plant by the polynucleotide of interest.

18. A method of producing seed comprising FACS sorting, culturing/optimizing and regeneration, culture plant, recover seed.
